(19) United States
(12) Patent Application Publication
(10) Pub. No.: US 2020/0116715 A1
(43) Pub. Date: Apr. 16, 2020

(54) IMMUNOSIGNATURES FOR DIFFERENTIAL DIAGNOSIS

(71) Applicant: HealthTell Inc., San Ramon, CA (US)

(72) Inventors: Robert William GERWIEN, San Ramon, CA (US); Theodore Michael TARASOW, San Ramon, CA (US); Jonathan Scott MELNICK, Walnut Creek, CA (US)

(21) Appl. No.: 16/624,284

(22) PCT Filed: Jun. 19, 2018
(86) PCT No.: PCT/US2018/038240

§ 371 Date: Dec. 18, 2019

Related U.S. Application Data
(60) Provisional application No. 62/581,581, filed on Nov. 3, 2017.
(60) Provisional application No. 62/522,636, filed on Jun. 20, 2017.
(60) Provisional application No. 62/522,052, filed on Jun. 19, 2017.

(51) Int. Cl. G01N 33/564; C07K 14/47; G16B 30/10

(52) U.S. Cl. CPC G01N 33/564; G16B 30/10; C07K 14/4713

(57) ABSTRACT

The disclosed embodiments concern methods, apparatus, and systems for providing differential diagnosis of autoimmune diseases including, Systemic Lupus Erythematosus (SLE) and Rheumatoid Arthritis (RA). The disclosed embodiments provide for differentially diagnosing autoimmune diseases from each other, from other autoimmune diseases, and from mimic disease conditions that are not classified as autoimmune, but that present with symptoms that are often associated with the autoimmune diseases.

### CROSS-REFERENCE

This application claims the benefit of U.S. Provisional Application Nos. 62/522,052, filed Jun. 19, 2017; 62/522,636, filed Jun. 20, 2017; and 62/581,581, filed Nov. 3, 2017, each of which is incorporated herein by reference in its entirety.

### BACKGROUND OF THE INVENTION

Detecting and diagnosing immune-mediated disorders, such as autoimmune disorders, is challenging, with patients having a difficult time receiving an accurate or correct diagnosis. In many instances, patients are often misdiagnosed with other autoimmune conditions because of the closely related nature of these diseases. There are currently no reliable bio-markers available for the detection and assessment of automimmune diseases or disorders.

### SUMMARY OF THE INVENTION

Disclosed herein are methods, apparatus, and systems for providing a differential diagnosis of an autoimmune disease in a subject having or being suspected of having an autoimmune disease. Additionally, the methods, apparatus, and systems are provided for identifying candidate biomarkers, including protein biomarkers useful for the diagnosis, prognosis, monitoring, activity and screening of autoimmune diseases, and/or as therapeutic targets for treatment of autoimmune diseases.

In some instances, methods, apparatus, systems, and kits are presented for providing differential diagnosis of autoimmune diseases including, Systemic Lupus Erythematosus (SLE), Rheumatoid Arthritis (RA), Sjogrens' disease (SS), Scleroderma, Osteoarthritis (OA), and Fibromyalgia (FM). The disclosed embodiments provide for differentially diagnosing autoimmune diseases from each other, and from mimic disease conditions that are not classified as autoimmune, but that present with symptoms that are often associated with certain autoimmune diseases. Non-limiting examples of mimic disease conditions include osteoarthritis and fibromyalgia, which overlap in symptomology with autoimmune diseases such as SLE and RA. Additionally, methods, apparatus, and systems are presented for providing differential diagnosis of autoimmune diseases including SLE and RA from samples obtained from a mixed population of conditions including other autoimmune diseases and non-auotimmune diseases. In some instances, the mixed population also includes samples from healthy subjects. Candidate biomarkers discriminating different conditions are also provided.

In one embodiment, disclosed herein are methods, apparatus, systems, and kits for making a differential diagnosis of an autoimmune disease, said method comprising: A method of making a differential diagnosis of an autoimmune disease, said method comprising: (a) contacting a sample from a subject to an array of peptides comprising at least 10,000 different peptides; (b) detecting the binding of antibodies present in said sample to at least 25 peptides on said array to obtain a combination of binding signals; and (c) comparing said combination of binding signals to one or more groups of combination of reference binding signals, wherein at least one of said group of combination of reference binding signals are obtained from a plurality of reference subjects known to have a disease different from the autoimmune disease of the subject to enable the differential diagnosis of said subject for the autoimmune disease, wherein the method performance is characterized by an area under the receiver operator characteristic (ROC) curve (AUC) between the autoimmune disease and each of the group of combinations of reference binding signals being greater than 0.6. In some instances, the methods, systems and apparatuses disclosed herein The method of claim 1, further comprises (i) identifying a combination of differentiating reference binding signals, wherein said differentiating reference binding signals distinguish samples from reference subjects known to have said autoimmune disease from samples from reference subjects known to have said disease different from the autoimmune disease; and (ii) applying the combination of differentiating reference binding signals to the methods, systems and apparatuses disclosed in step 1(c), above, to enable differential diagnosis of the autoimmune diseases. In yet other instances, each of said combination of differentiating reference binding signals is obtained by detecting the binding of antibodies present in a sample from each of said plurality of reference subjects to said at least 25 peptides on an array of peptides comprising at least 10,000 different peptides in step (a) of the methods, systems and apparatuses disclosed above.

In some instances, the methods, apparatus, systems, and kits disclosed further comprises comparing said combination of binding signals to a reference binding signal obtained from a plurality of reference subjects known to have the autoimmune disease. In some instances, the different disease is an autoimmune disease and/or is a non-autoimmune mimic disease. In some instances, the autoimmune disease is systemic lupus erythematosus, and the different autoimmune disease is rheumatoid arthritis, exemplified in discriminating peptides that are enriched by greater than 100% in one or more sequence motifs listed in FIG. 7A and/or in one or more amino acids as listed in FIG. 7B.

In some instances, the autoimmune disease is systemic lupus erythematosus, and the different non-autoimmune-disease is osteoarthritis, exemplified in discriminating peptides that are enriched by greater than 100% in one or more sequence motifs as listed in FIG. 8A, and/or in one or more amino acids listed in FIG. 8B.

In some instances, the autoimmune disease is systemic lupus erythematosus, and the different non-autoimmune disease is fibromyalgia, exemplified in discriminating peptides that are enriched by greater than 100% in one or more sequence motifs listed in FIG. 9A, and/or in one or more amino acids listed in FIG. 9B.

In some instances, autoimmune disease is systemic lupus erythematosus, and the different autoimmune disease is Sjogren's disease, exemplified in discriminating peptides that are enriched by greater than 100% in one or more sequence motifs listed in FIG. 10A, and/or in one or more amino acids listed in FIG. 10B.

In some instances, the autoimmune disease is systemic lupus erythematosus, and the different disease is a group of autoimmune diseases and non-autoimmune mimic diseases, exemplified in discriminating peptides that are enriched by greater than 100% in one or more sequence motifs listed in FIG. 5A, and/or in one or more amino acids listed in FIG. 5B.

In some instances, the said autoimmune disease is systemic lupus erythematosus, and the different disease is a group of autoimmune diseases, non-autoimmune mimic diseases, and healthy controls, exemplified in discriminating peptides that are enriched by greater than 100% in one or more sequence motifs listed in FIG. 6A, and/or in one or more amino acids listed in FIG. 6B.

In some instances, the methods, systems and apparatuses further comprises comparing the binding signal from subjects with systemic lupus erythematosus to a combination of reference binding signals obtained from healthy subjects, exemplified in said discriminating peptides that are enriched by greater than 100% in one or more sequence motifs listed in FIG. 4A, and/or in one or more amino acids listed in FIG. 4B.

In some instances, the autoimmune disease is rheumatoid arthritis (RA), and said different disease is OA, exemplified in discriminating peptides that are enriched by greater than 100% in one or more sequence motifs listed in FIG. 22A, and/or in one or more amino acids listed in FIG. 22B.

In some instances, the autoimmune disease is rheumatoid arthritis (RA), and said different disease is FM, exemplified in discriminating peptides that are enriched by greater than 100% in one or more sequence motifs listed in FIG. 23A, and/or in one or more amino acids listed in FIG. 23B.

In some instances, the autoimmune disease is rheumatoid arthritis (RA), and said different disease is SS, exemplified in discriminating peptides that are enriched by greater than 100% in one or more sequence motifs listed in FIG. 24A, and/or in one or more amino acids listed in FIG. 24B.

In some instances, the autoimmune disease is RA, and said different disease is a group of autoimmune diseases and non-autoimmune mimic diseases, exemplified in discriminating peptides that are enriched by greater than 100% in one or more sequence motifs listed in FIG. 21A, and/or in one or more amino acids listed in FIG. 21B.

In some instances, the autoimmune disease is RA, and said different disease is a group of autoimmune diseases, non-autoimmune mimic diseases, and healthy controls, exemplified in discriminating peptides that are enriched by greater than 100% in one or more sequence motifs listed in FIG. 20A, and/or in one or more amino acids listed in FIG. 20B.

In some instances, the methods, apparatus, systems, and kits further comprises comparing the binding signal from subjects with RA to a combination of reference binding signals obtained from healthy subjects (HC), exemplified in discriminating peptides that are enriched by greater than 100% in one or more sequence motifs listed in FIG. 18A, and/or in one or more amino acids listed in FIG. 18B.

In some instances, the methods, apparatus, systems, and kits further comprises combining differentiating binding signals that distinguish samples from each of SLE, RA, FM, OA, SS and HC from each other to obtain a multiclass set of discriminating peptides that simultaneously distinguish each condition from each other, exemplified in discriminating peptides that are enriched by greater than 100% in one or more sequence motifs listed in FIG. 30A, and/or in one or more amino acids listed in FIG. 30B.

Also disclosed herein are methods, apparatus, systems, and kits for identifying at least one candidate biomarker for autoimmune diseases, including systemic lupus erythematosus (SLE) and rheumatoid arthritis (RA).

Additionally disclosed herein are methods, apparatus, systems, and kits for identifying at least one candidate biomarker for an autoimmune disease, the method comprising (a) providing a peptide array and incubating a biological sample from a plurality of reference subjects known to have the autoimmune disease to the peptide array; (b) identifying a set of discriminating peptides bound to antibodies in the biological sample from said subject, the set of discriminating peptides displaying binding signals capable of differentiating the autoimmune disease from samples from healthy subjects; (c) querying a proteome database with each of the peptides in the set of discriminating peptides; (d) aligning each of the peptides in the set of discriminating peptides to one or more proteins in the human proteome database; and (e) obtaining a relevance score and ranking for each of the identified proteins from the proteome database, wherein each of the identified proteins is a candidate biomarker for the autoimmune disease.

In still other embodiments of the methods, apparatus, systems, and kits disclosed herein, the sample is a blood sample. In some instances, the blood sample is selected from whole blood, plasma, or serum. In other instances, the sample is a serum sample. In yet other instances, the sample is a plasma sample. In still other instances, the sample is a dried blood sample. In still other embodiments, the array of peptides comprises at least 50,000 different peptides. In some embodiments, the peptide array comprises at least 300,000 different peptides. In other embodiments, the peptide array comprises at least 500,000 different peptides. In yet other instances, the peptide array comprises at least 2,000,000 different peptides. In still other embodiments, the peptide array comprises at least 3,000,000 different peptides. In yet other instances, the different peptides on the peptide array is at least 5 amino acids in length. In still other instances, the different peptides on the peptide array are between 5 and 13 amino acids in length. In some other instances, the different peptides are synthesized from less than 20 amino acids. In some other instances, the different peptides on the array are deposited. In still other embodiments, the different peptides on the array are synthesized in situ.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings in the following.

FIG. 1 shows the detection of antibody-bound array peptides of immunosignatures.

FIG. 2 shows a schematic of an exemplary peptide array for use in the disclosed embodiments.

FIG. 3 shows the support vector machines (SVM) process of 5-fold cross validation.

FIG. 4A and FIG. 4B show peptide motifs (FIG. 4A) and amino acids (FIG. 4B) that are enriched in the peptides that discriminate between the systemic lupus erythematosus (SLE) samples from the healthy donor (HC) samples.

FIG. 5A and FIG. 5B show peptide motifs (FIG. 5A) and amino acids (FIG. 5B) that are enriched in the peptides that discriminate between the SLE samples from a group of other diseases that are autoimmune and non-autoimmune-mimic diseases (Other AI+non-AI mimic).

FIG. 6A and FIG. 6B shows peptide motifs (FIG. 6A) and amino acids (FIG. 6B) that are enriched in the peptides that discriminate between the SLE samples from the “Not SLE” samples, which are samples of other autoimmune diseases, non-autoimmune mimic diseases and healthy controls.

FIG. 7A and FIG. 7B show peptide motifs (FIG. 7A) and amino acids (FIG. 7B) that are enriched in the peptides that discriminate between the SLE samples from the rheumatoid arthritis (RA) group of samples.

FIG. 8A and FIG. 8B show peptide motifs (FIG. 8A) and amino acids (FIG. 8B) that are enriched in the peptides that discriminate between the SLE samples from the osteoarthritis (OA) group of samples.

FIG. 9A and FIG. 9B show peptide motifs (FIG. 9A) and amino acids (FIG. 9B) that are enriched in the peptides that discriminate between the SLE samples from the fibromyalgia (FM) group of samples.

FIG. 10A and FIG. 10B show the peptide motifs (FIG. 10A) and amino acids (FIG. 10B) that are enriched in the peptides that discriminate between the SLE samples from the Sjogren's (SS) group of samples.

FIG. 11A shows a Volcano plot visualizing library peptides displaying antibody-binding signals that significantly differentiate SLE samples from samples from Healthy Donors.

FIG. 11B shows a Volcano plot visualizing library peptides displaying antibody-binding signals that significantly differentiate SLE samples from samples of subjects of the “Other AI+non-AI mimic” group.

FIG. 11C shows a Volcano plot visualizing library peptides displaying antibody-binding signals that significantly differentiate SLE samples from samples of subjects of the “Not SLE” group.

FIG. 12 shows a Venn diagram showing the distribution of peptides that passed the Bonferroni cutoff for each of contrasts and the 478 peptides that are common to all contrasts, i.e., SLE vs. other AI+non-AI mimic diseases+healthy, shown as “NOT SLE”; SLE vs. Healthy; and SLE vs other AI+non-AI mimic diseases, shown as “Other AI”.

FIG. 13 shows graphs of the 5-fold cross validated performance at a 95% confidence level (Y-axis) as a function of the number of input discriminating peptides (Number of Features i.e. petides; x-axis) in a SLE Healthy Donor assay.

FIG. 14 shows the area under the receiver operating characteristic curve (AUC) as assay performance in discriminating SLE samples from HC (Healthy), from Other AI+non-AI mimic (Other AI), and from the “Not SLE” group i.e. Other AI+non-AI mimic+HC. In each group, the bar on the left represents performance in discriminating SLE alone from the indicated condition, and the bar on the right represents performance in discriminating a mixture of Mixed SLE and Other AI samples.

FIG. 15 shows assay performance for differential diagnosis of SLE from RA, Sjogrens, OA, and Fibromyalgia.

FIG. 16 shows the assay performance using a multiclassifier that simultaneously discriminates each disease from a mixture of the remaining others.

FIG. 17A, FIG. 17B, and FIG. 17C show the top candidate biomarkers identified by peptides discriminating SLE from healthy subjects (FIG. 17A), from a group of subjects with other autoimmune disease or autoimmune-mimic diseases (Other AI+non-AI mimic) (FIG. 17B), and from the “Not SLE” group represented by samples from Other AI+non-AI mimic and HC (FIG. 17C).

FIG. 18A and FIG. 18B show peptide motifs (FIG. 18A) and amino acids (FIG. 18B) that are enriched in the peptides that discriminate between the RA samples from the healthy donor (HC) samples.

FIG. 19A and FIG. 19B show peptide motifs (FIG. 19A) and amino acids (FIG. 19B) that are enriched in the peptides that discriminate between the RA samples from the samples from other rheumatic diseases.

FIG. 20A and FIG. 20B shows peptide motifs (FIG. 20A) and amino acids (FIG. 20B) that are enriched in the peptides that discriminate between the RA samples from the “Not RA” group represented by samples from Other AI+non-AI mimic and HC (C).

FIG. 21A and FIG. 21B show peptide motifs (FIG. 21A) and amino acids (FIG. 21B) that are enriched in the peptides that discriminate between the RA samples from Other AI+non-AI mimic group.

FIG. 22A and FIG. 22B show peptide motifs (FIG. 22A) and amino acids (FIG. 22B) that are enriched in the peptides that discriminate between the RA samples from the OA group of samples.

FIG. 23A and FIG. 23B shows peptide motifs (FIG. 23A) and amino acids (FIG. 23B) that are enriched in the peptides that discriminate between the RA samples from the FM group of samples.

FIG. 24A and FIG. 24B shows peptide motifs (FIG. 24A) and amino acids (FIG. 24B) that are enriched in the peptides that discriminate between the RA samples from the SS group of samples.

FIG. 25A shows a Volcano plot visualizing library peptides displaying antibody-binding signals that significantly differentiate RA samples from samples from Healthy Donors.

FIG. 25B shows a Volcano plot visualizing library peptides displaying antibody-binding signals that significantly differentiate RA samples from samples of subjects of the “Other AI+non-AI mimic” group.

FIG. 25C shows a Volcano plot visualizing library peptides displaying antibody-binding signals that significantly differentiate RA samples from samples of subjects of the “Not RA” group.

FIG. 26 shows a Venn diagram showing the distribution of peptides that passed the Bonferroni cutoff for each of contrasts and the 491 peptides that are common to all contrasts, i.e. RA vs. other AI+non-AI mimic diseases+healthy, shown as “NOT RA”; RA vs. Healthy; and RA vs. other AI+non-AI mimic diseases, shown as “Other AI”.

FIG. 27 shows the area under the receiver operating characteristic curve (AUC) as assay performance in discriminating RA samples from HC, from Other AI+non-AI mimic, and from “Not RA” i.e. Other AI+non-AI mimic+HC. In each group, the bar on the left represents performance in discriminating RA alone from the indicated condition, and the bar on the right represents performance in discriminating a mixture of Mixed RA and Other AI samples.

FIG. 28 shows assay performance for differential diagnosis of RA from SLE, Sjogrens, OA, and Fibromyoalgia.

FIG. 29A, FIG. 29B, and FIG. 29C show candidate biomarkers identified by peptides discriminating RA from healthy subjects (FIG. 29A), RA from a group of subjects with other autoimmune disease (Other AI+non-AI mimic diseases) (FIG. 29B), and RA from the “Not RA” group represented by samples from Other AI+non-AI mimic and HC (FIG. 29C).

FIG. 30A and FIG. 30B shows peptide motifs (FIG. 30A) and amino acids (FIG. 30B) that are enriched in the peptides that simultaneously discriminate SLE, RA, FM, OA, SS, and HC from each other.

FIG. 31 shows a heat map visualizing the probabilities of SLE, RA, FM, OA, SS, and HC class assignments. Each sample has a predicted class membership for each disease class ranging from 0 (black) to 100% (white).

FIG. 32 shows the top significant peptides that discriminate between the SLE samples from the healthy (HC) group of samples.

FIG. 33 shows the top significant peptides that discriminate between the SLE samples from the Other Autoimmune and non-Autoimmune mimic diseases (Other AI+non-AI) group of samples.

FIG. 34 shows the top significant peptides that discriminate between the SLE samples from the Not SLE (Not SLE—Other AI+non-AI+HC) group of samples.

FIG. 35 shows the top significant peptides that discriminate between the RA samples from the healthy (HC) group of samples.

FIG. 36 shows the top significant peptides that discriminate between the RA samples from the Other Autoimmune and non-Autoimmune mimic diseases (Other AI+non-AI) group of samples.

FIG. 37 shows the top significant peptides motifs that discriminate between the RA samples from the Not RA (Not RA=Other AI+non-AI+HC) group of samples.

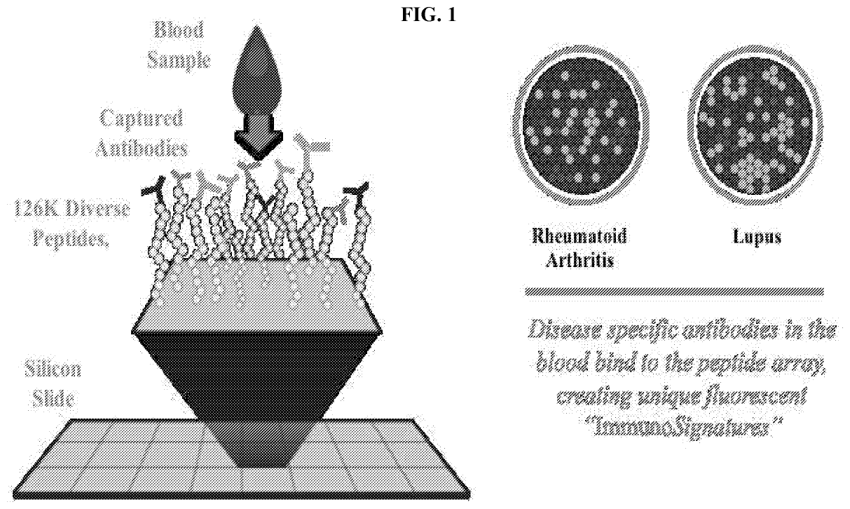

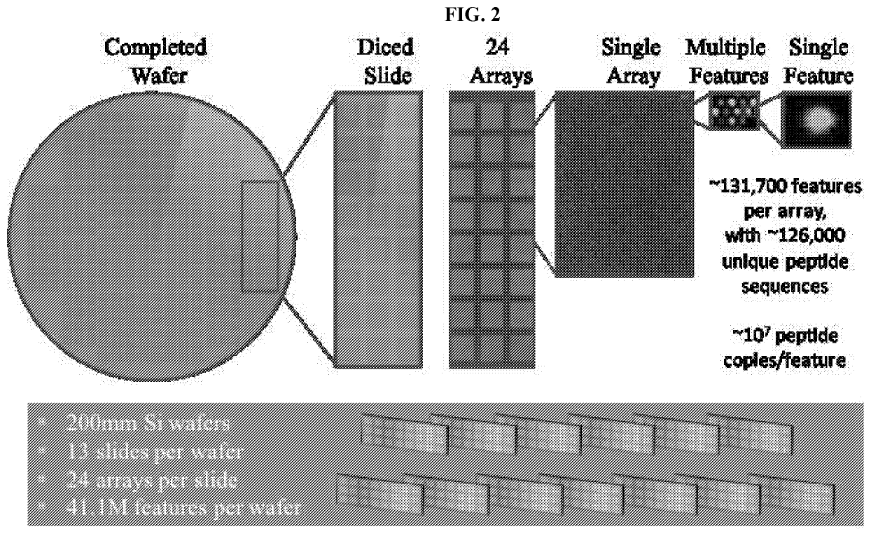

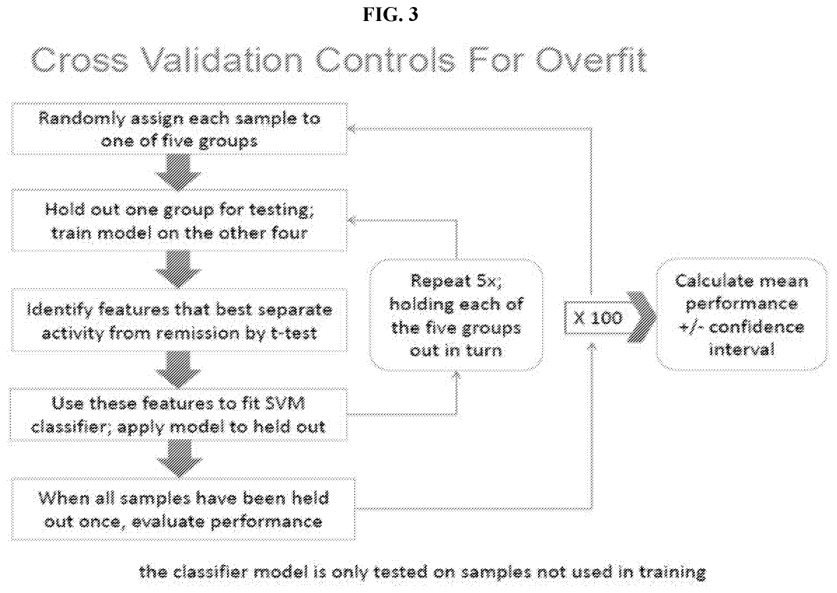

### DETAILED DESCRIPTION OF THE INVENTION

Detecting and diagnosing immune-mediated disorders, such as autoimmune disorders, is challenging, with patients having a difficult time receiving an accurate or correct diagnosis. In many instances, patients are often misdiagnosed with other autoimmune conditions because of the closely related nature of these diseases. There are currently no reliable bio-markers available for the detection and assessment of automimmune diseases or disorders.

Systemic lupus erythematosus (SLE) and rheumatoid arthritis (RA) are examples of autoimmune diseases that may attack the body's cells and tissues, resulting in inflammation and tissue damage. The development processes and mechanisms of SLE and RA are not well established. Due to the similarity of symptoms the differential diagnosis and treatment of these two diseases is challenging. Additionally, patients with SLE or RA can have more than one disease at the same time i.e. an overlap disease or condition that can be an autoimmune disease, or a non-autoimmune condition that mimics the symptoms of the disease. Consequently, a correct diagnosis for such a disease can take several months or even years to determine. It typically requires a combination of medical history review, multiple physical examinations, numerous lab tests, and often scans. Clinically, laboratory tests may be performed using various serum markers. However, the markers are nonspecific and their roles in the differential diagnosis, prediction and disease activity evaluation of SLE and RA are not well known.

The disclosed embodiments concern methods, apparatus, systems, and kits for providing differential diagnosis of autoimmune diseases including, Systemic Lupus Erythematosus (SLE) and Rheumatoid Arthritis (RA). The disclosed embodiments provide for differentially diagnosing autoimmune diseases from each other, and from mimic disease conditions that are not classified as autoimmune, but that present with symptoms that are often associated with the autoimmune diseases. Non-limiting examples of non-autoimmune mimic disease/conditions include osteoarthritis and fibromyalgia.

Methods, apparatus, systems, and kits are provided herein that enable differential diagnosis of autoimmune disorders (AIs) using a single noninvasive screening method that identifies differential patterns of peripheral-blood antibody binding to peptide arrays. Differential binding of patient samples to peptide arrays results in specific binding patterns, i.e. immunosignatures (IS), that are indicative of the health condition, e.g. autoimmune disease, of the patient. Additionally, the apparatus and systems provided herein allow for the identification of antigens or binding partners to antibodies of the biological sample, which can be assessed as candidate biomarkers for targeted therapeutic interventions.

Typically, an immunosignature characteristic of a condition is determined relative to one or more reference immunosignatures, which are obtained from one or more different sets of reference samples, each set being obtained from one or more groups of reference subjects, each group having a different condition, e.g. a different autoimmune disease (AI). For example, an immunosignature obtained from a test subject identifies the AI of the test subject when compared to immunosignatures of reference subjects having a different AI, subjects with a non-AI disease or condition that mimics the symptoms of the AI being tested, subjects having overlap disease comprising a different AI and/or mimic conditions. Accordingly, comparison of immunosignatures from a test subject with those of reference subjects can provide a differential diagnosis for the AI. A reference group can be a group of healthy subjects, and the condition is referred to herein as a healthy condition.

The methods, apparatus, systems, and kits provided can detect a number of different AI diseases in samples, e.g. blood, from different individuals within a population of subjects that are suspected or known to have an AI disease. In some embodiments, the IS is based on diverse yet reproducible patterns of antibody binding to an array of peptides that are selected to provide an unbiased sampling of at least a portion of amino acid combinations less than 20 amino acids rather than represent known proteomic sequences. A peptide bound by an antibody in a sample from a subject may not be the natural target sequence, but may instead mimic the sequence or structure of the cognate natural epitope. In some embodiments, the peptide bound by an antibody may be at least partially identical, similar to or otherwise related to a known proteomic sequence. For example, none of the peptides in the IST library described in Example 1 are identical matches to any 9 mer sequence in known proteome databases. This is not surprising since the number of possible 9 mer peptide sequences is several orders of magnitude greater than the number of contiguous 9 mer sequences in the proteome databases. Accordingly, the probability of any mimetic-peptide corresponding exactly to a natural sequence is low. Each IS peptide sequence that is selectively bound by an antibody could be a functional surrogate of the epitope that the antibody recognized in vivo. Consequently, the sequences of proteins comprising part or all of the antibody-bound array peptide sequence can serve to identify candidate protein biomarkers, which can be assessed as therapeutic targets.

In one aspect, a method is provided for the differential diagnosis of an autoimmune disease e.g. SLE or RA comprising: (a) contacting a sample from a subject to an array of peptides comprising at least 10,000 different peptides; (b) detecting the binding of antibodies present in said sample to at least 25 peptides on said array to obtain a combination of binding signals; and (c) comparing said combination of binding signals to one or more groups of combinations of reference binding signals, wherein at least one of each of said group of combinations of reference binding signals are obtained from a plurality of reference subjects known to have a disease different from the autoimmune disease of the subject, thereby enabling the differential diagnosis of said subject for the autoimmune disease, wherein the method performance is characterized by an area under the receiver operator characteristic (ROC) curve (AUC) between the autoimmune disease and each of the group of combinations of reference binding signals being greater than 0.6. In some instances, the methods, systems and apparatuses further comprise (i) identifying a combination of differentiating binding signals, wherein said differentiating reference binding signals distinguish samples from reference subjects known to have said autoimmune disease from samples from reference subjects known to have said disease different from the autoimmune disease; and (ii) identifying a combination of discriminating peptides, wherein said combination of differentiating reference binding signals correspond to the combination of discriminating peptides. In some embodiments, reference subjects include subjects having a different AI, subjects with a non-AI disease or condition that mimics the symptoms of the AI being tested, subjects having overlap disease comprising a different AI and/or mimic conditions. In other embodiments, the reference subjects are healthy subjects. The array peptides can be deposited or can be synthesized in situ on a solid surface. In some embodiments, the method performance can be characterized by an area under the receiver operator characteristic (ROC) curve (AUC) being greater than 0.6.

In some embodiments, the method further comprises identifying a combination of differentiating reference binding signals that distinguish AI samples e.g. SLE or RA, from one or more reference subjects known to have a different disease, and identifying the combination of the array peptides that display the combination of differentiating binding signals i.e. discriminating peptides, wherein the combination of differentiating reference binding signals corresponds to the combination of discriminating peptides. The combination of differentiating binding signals can comprise signals that are increased or decreased, newly added signals, and/or signals that are lost in the presence of an AI disease relative to the corresponding binding signals obtained from reference samples. The array peptides that display the combination of differentiating binding signals are known as discriminating peptides. The term “discriminating” when used in reference to array peptides is used herein interchangeably with “classifying”. In some embodiments, a combination of differentiating reference binding signals comprises a combination of binding signals to at least 1, at least 2, at least 5, at least 10, at least 15, at least 20, at least 25, at least 30, at least 35, at least 40, at least 45, at least 50, at least 60, at least 70, at least 80, at least 90, at least 100, at least 125, at least 150, at least 175, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, at least 10000, at least 20000, or more discriminating peptides on an array. For example, at least 25 peptides on an array of 10,000 peptides are identified as discriminating peptides for a given condition. In some embodiments, each combination of differentiating binding signals is obtained by detecting the binding of antibodies present in a reference sample from each of a plurality of reference subjects to at least 25 peptides on same arrays of peptides comprising at least 10,000 different peptides. In some embodiments, the peptides are synthesized in situ. In some embodiments, discriminating peptides are identified from antibodies binding differentially to peptide arrays comprising a library of at least 5,000, at least 10,000, at least 15,000, at least 20,000, at least 25,000, at least 50,000, at least 100,000, at least 200,000, at least 300,000, at least 400,000, at least 500,00, at least 1,000,000, at least 2,000,000, at least 3,000,000, at least 4,000,000, at least 5,000,000 or at least 100,000,000 or more different peptides on the array substrate.

### Kits

In another aspect, a kit is provided for conducting the differential diagnosis of an autoimmune disease e.g. SLE or RA, the kit comprising a peptide array and means for assaying a sample from a subject on the peptide array and detecting signals on the peptide array, comprising: (a) contacting a sample from a subject to an array of peptides comprising at least 10,000 different peptides; (b) detecting the binding of antibodies present in said sample to at least 25 peptides on said array to obtain a combination of binding signals; and (c) comparing said combination of binding signals to one or more groups of combinations of reference binding signals, wherein at least one of each of said group of combinations of reference binding signals are obtained from a plurality of reference subjects known to have a disease different from the autoimmune disease of the subject, thereby enabling the differential diagnosis of said subject for the autoimmune disease, wherein the kit performance is characterized by an area under the receiver operator characteristic (ROC) curve (AUC) between the autoimmune disease and each of the group of combinations of reference binding signals being greater than 0.6. In some instances, the kit further comprises means for (i) identifying a combination of differentiating binding signals, wherein said differentiating reference binding signals distinguish samples from reference subjects known to have said autoimmune disease from samples from reference subjects known to have said disease different from the autoimmune disease; and (ii) identifying a combination of discriminating peptides, wherein said combination of differentiating reference binding signals correspond to the combination of discriminating peptides. In some embodiments, reference subjects include subjects having a different AI, subjects with a non-AI disease or condition that mimics the symptoms of the AI being tested, subjects having overlap disease comprising a different AI and/or mimic conditions. In other embodiments, the reference subjects are healthy subjects. The array peptides can be deposited or can be synthesized in situ on a solid surface. In some embodiments, the kit performance can be characterized by an area under the receiver operator characteristic (ROC) curve (AUC) being greater than 0.6.

In some embodiments, the kit further comprises identifying a combination of differentiating reference binding signals that distinguish AI samples e.g. SLE or RA, from one or more reference subjects known to have a different disease, and identifying the combination of the array peptides that display the combination of differentiating binding signals i.e. discriminating peptides, wherein the combination of differentiating reference binding signals corresponds to the combination of discriminating peptides. The combination of differentiating binding signals can comprise signals that are increased or decreased, newly added signals, and/or signals that are lost in the presence of an AI disease relative to the corresponding binding signals obtained from reference samples. The array peptides that display the combination of differentiating binding signals are known as discriminating peptides. The term “discriminating” when used in reference to array peptides is used herein interchangeably with “classifying”. In some embodiments, a combination of differentiating reference binding signals comprises a combination of binding signals to at least 1, at least 2, at least 5, at least 10, at least 15, at least 20, at least 25, at least 30, at least 35, at least 40, at least 45, at least 50, at least 60, at least 70, at least 80, at least 90, at least 100, at least 125, at least 150, at least 175, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, at least 10000, at least 20000, or more discriminating peptides on an array. For example, at least 25 peptides on an array of 10,000 peptides are identified as discriminating peptides for a given condition. In some embodiments, each combination of differentiating binding signals is obtained by detecting the binding of antibodies present in a reference sample from each of a plurality of reference subjects to at least 25 peptides on same arrays of peptides comprising at least 10,000 different peptides. In some embodiments, the peptides are synthesized in situ. In some embodiments, discriminating peptides are identified from antibodies binding differentially to peptide arrays comprising a library of at least 5,000, at least 10,000, at least 15,000, at least 20,000, at least 25,000, at least 50,000, at least 100,000, at least 200,000, at least 300,000, at least 400,000, at least 500,00, at least 1,000,000, at least 2,000,000, at least 3,000,000, at least 4,000,000, at least 5,000,000 or at least 100,000,000 or more different peptides on the array substrate.

In some embodiments, at least 0.00005%, at least 0.0001%, at least 0.0005%, at least 0.0001%, at least 0.001%, at least 0.003%, at least 0.005%, at least 0.01%, at least 0.05%, at least 0.1%, at least 0.5%, at least 1%, at least 0.5%, at least 1.5%, at least 2%, at least 3%, at least 4%, at least 5%, at least 10%, at least 25%, at least 50%, at least 75%, at least 80%, or at least 90%, of the total number of peptides on an array are discriminating peptides. In other embodiments, all of the peptides on an array are discriminating peptides.

### Binding Assay

The immunosignature of a subject is identified as a pattern of binding of antibodies that are bound to the array peptides. The peptide array can be contacted with a sample e.g. blood, plasma or serum, under any suitable conditions to promote binding of antibodies in the sample to peptides immobilized on the array. Thus, the methods and kits of the invention are not limited by any specific type of binding conditions employed. Such conditions will vary depending on the array being used, the type of substrate, the density of the peptides arrayed on the substrate, desired stringency of the binding interaction, and nature of the competing materials in the binding solution. In a preferred embodiment, the conditions comprise a step to remove unbound antibodies from the addressable array. Determining the need for such a step, and appropriate conditions for such a step, are well within the level of skill in the art.

Any suitable detection technique can be used in the methods, apparatuses, systems, and kits described herein for detecting binding of antibodies in a sample to peptides on the array to generate an immune profile consequent to an AI. In one embodiment, any type of detectable label can be used to label peptides on the array, including but not limited to radioisotope labels, fluorescent labels, luminescent labels, and electrochemical labels (i.e.: ligand labels with different electrode mid-point potential, where detection comprises detecting electric potential of the label). Alternatively, bound antibodies can be detected, for example, using a detectably labeled secondary antibody.

Detection of signal from detectable labels is well within the level of skill in the art. For example, fluorescent array readers are well known in the art, as are instruments to record electric potentials on a substrate (For electrochemical detection see, for example, J. Wang (2000) Analytical Electrochemistry, Vol., 2nd ed., Wiley—VCH, New York). Binding interactions can also be detected using other label-free methods such a s SPR and mass spectrometry. SPR can provide a measure if dissociation constants and dissociation rates. The A-100 Biocore/GE instrument, for example, is suitable for this type of analysis. FLEX chips can be used to up to 400 binding reactions on the same support.

Alternatively, binding interactions between antibodies in a sample and the peptides on an array can be detected in a competition format. A difference in the binding profile of an array to a sample in the presence versus absence of a competitive inhibitor of binding can be useful in characterizing the sample.

### Classification Algorithms

Analyses of the antibody binding signal data i.e. immunosignaturing (IS), and the differential diagnosis derived therefrom are typically performed using various algorithms and programs. The antibody binding pattern produced by the labeled secondary antibody bound to primary antibodies is scanned using, for example, a laser scanner. The images of the binding signals acquired by the scanner can be imported and processed using software such as the GenePix Pro 8 software (Molecular Devices, Santa Clara, Calif.), to provide tabular information for each peptide, for example, in a continuous value ranging from 0-65,535. Tabular data can be imported and statistical analysis performed using, for example, into the R language and environment for statistical computing (R Foundation for Statistical Computing, Vienna, Austria. URL https://www.R-project.org/).

Peptides displaying differential signaling patterns, i.e. discriminating peptides, between samples obtained from reference subjects e.g. healthy subjects or subjects with a different disease, can be identified using known statistical tests such as a Student's T-test or ANOVA. The statistical analyses are applied to select the discriminating peptides that distinguish the different conditions at predetermined stringency levels. In some embodiments, a list of the most discriminating peptides can be obtained by ranking the peptides by statistical means such as theirp-value. For example, discriminating peptides can be ranked and identified as having p-values of between zero and one. The cutoff for the p-value can be further adjusted to account for instances when several dependent or independent statistical tests are being performed simultaneously on a single data set. For example, a Bonferroni correction can be used to reduce the chances of obtaining false positives when multiple pairwise tests are performed on a single set of data. The correction is dependent on the size of the array library. In some embodiments, the cutoffp-value for determining the discriminating can be adjusted to less than 10⁻²⁰, less than 10⁻¹⁹, less than 10⁻¹⁸, less than 10⁻¹⁷, less than 10⁻¹⁶, less than 10⁻¹⁵, less than 10⁻¹⁴, less than 10⁻¹³, less than 10⁻¹², less than 10⁻¹¹, less than 10⁻¹⁰, less than 10⁻⁹, less than 10⁻⁸, less than 10⁻⁷, less than 10⁻⁶, or less than 10⁻⁵, or less than 10⁻⁴, or less than 10⁻³, or less than 10⁻². The adjustment is dependent on the size of the array library. Alternatively, discriminating peptides are not ranked, and the binding signal information displayed up to all of the identified discriminating peptides is used to classify a condition e.g. the serological state of a sample.

Subsequently, binding signal information of the discriminating peptides selected following statistical analysis can be subsequently imported into a machine learning algorithm to obtain a statistical or mathematical model i.e. a classifier, that classifies the antibody profile data with accuracy, sensitivity and specificity, and determines the serological state of a sample, and other applications described elsewhere herein. Any one of the many computational algorithms can be utilized for the classification purposes.

The classifiers can be rule-based or can be computationally intelligent. Further, the computationally intelligent classification algorithms can be supervised or unsupervised. A basic classification algorithm, Linear Discriminant Analysis (LDA) may be used in analyzing biomedical data in order to classify two or more disease classes. LDA can be, for example, a classification algorithm. A more complex classification method, Support Vector Machines (SVM), uses mathematical kernels to project the original predictors to higher-dimensional spaces, then identifies the hyperplane that optimally separates the samples according to their class. Some common kernels include linear, polynomial, sigmoid or radial basis functions. A comparative study of common classifiers described in the art is described in (Kukreja et al, BMC Bioinformatics. 2012; 13: 139). Other algorithms for data analysis and predictive modeling based on data of antibody binding profiles include but are not limited to Naive Bayes Classifiers, Logistic Regression, Quadratic Discriminant Analysis, K-Nearest Neighbors (KNN), K Star, Attribute Selected Classifier (ACS), Classification via clustering, Classification via Regression, Hyper Pipes, Voting Feature Interval Classifier, Decision Trees, Random Forest, and Neural Networks, including Deep Learning approaches.

In some embodiments, antibody binding profiles are obtained from a training set of samples, which are used to identify the most discriminative combination of peptides by applying an elimination algorithm based on SVM analysis. The accuracy of the algorithm using various numbers of input peptides ranked by level of statistical significance can be determined by cross-validation. To generate and evaluate antibody binding profiles of a feasible number of discriminating peptides, multiple models can be built, using a plurality of discriminating peptides to identify the best performing model. While the method does not exclude limiting the number of peptides, the method can exploit all or substantially all available peptide binding information e.g. binding signals. Thus, the method contrasts with approaches that attempt to determine a priori the peptides whose sequences can be utilized for binding purposes. In some embodiments, up to all of the peptides on the array are discriminating peptides. In some embodiments, at least 25, at least 50, at least 75, at least 100, at least 200, at least 300, at least 400, at least 500, at least 750, at least 1000, at least 1500, at least 2000, at least 3000, at least 4000, at least 5000, at least 6000, at least 7000, at least 8000, at least 9000, at least 10,000, at least 11,000 at least 12,000 at least 13,000 at least 14,000 at least 15,000 at least 16,000 at least 17,000 at least 18,000 at least 19,000 at least 20,000 or more discriminating peptides are used to train a specific disease-classifying model. In some embodiments at least 0.00001%, at least 0.0001%, at least 0.0005%, at least 0.001%, at least 0.005%, at least 0.01%, at least 0.05%, at least 0.1%, at least 0.5%, at least 1.0%, at least 2%, at least 3%, at least 4%, at least 5%, at least 10%, at least 20%, at least 30%, at least 40%, at least 50%, at least 60%, at least 70%, at least 80%, at least 90%, at least 95%, or at least 99% of the total number of peptides on the array are discriminating peptides, and the corresponding binding signal information is used to train a specific condition-classifying model. In some embodiments, the signal information obtained for all of the peptides on the array is used to train the condition-specific model.

Multiple models comprising different numbers of discriminating peptides can be generated, and the performance of each model can be evaluated by a cross-validation process. An SVM classifier can be trained and cross-validated by assigning each sample of a training set of samples to one of a plurality of cross-validation groups. For example, for a five-fold cross-validation, each sample is assigned to one of four cross-validation groups such that each group comprises test and control i.e. reference samples; one of the cross-validation groups e.g. group 1, is held-out, and an SVM classifier model is trained using the samples in groups 2-4 (FIG. 3). Peptides that discriminate test cases and reference samples in the training group are analyzed and ranked, for example by statistical p-value; the top k peptides are then used as predictors for the SVM model. To elucidate the relationship between the number of input predictors and model performance, and to guard against overfitting, the sub=loop is repeated for a range of k, e.g. 25, 50, 100, 250, 1000, 200, 3000 top peptides or more. Predictions i.e. classification of samples in group 1 are made using the model generated using groups 2-4. Models for each of the four groups are generated, and the performance (AUC, sensitivity and/or specificity) is calculated using all the predictions from the 4 models using signal binding data from true disease samples. The cross-validation steps are repeated at least 100 times, and the average performance is calculated relative to a confidence interval e.g. 95%. Diagnostic visualization can be generated using e.g. model performance relative to the number of input peptides.

An optimal model/classifier based on antibody binding information to a set of discriminating input peptides (list of the most discriminating peptides, k) is selected and used to predict the disease status of a test set. The performance of different classifiers is determined using a validation set, and using a test set of samples, performance characteristics such as accuracy, sensitivity, specificity, and Area Under the Curve (AUC) of the Receiver Operating Characteristic (AUC) curve are obtained from the model having the greatest performance. In some embodiments, different sets of discriminating peptides are identified to distinguish different conditions. Accordingly, an optimal model/classifier based on a set of the most discriminating input peptides is established for each of the AI diseases to provide a differential diagnosis.

### Classification of Conditions

In some embodiments, individual binary classifiers can be obtained to differentially diagnose an AI relative to a different disease. For example, as shown in Example 3, an optimal classifier based on a combination of discriminating peptides is selected to differentially diagnose SLE from reference subjects having a different AI, subjects with a non-AI disease or condition that mimics the symptoms of the AI being tested, subjects having overlap disease comprising a different AI and/or mimic conditions.

In Example 2, the discriminating peptides were determined to provide a differential diagnosis of SLE by distinguishing samples from subjects with SLE from reference samples from different groups of subjects as shown in Table 1.

The characteristics of the combination of the discriminating peptides include the prevalence of one or more amino acids, and/or the prevalence of specific sequence motifs present in the identified discriminating peptides. Enrichment of amino acid and motif content is relative to the corresponding total amino acid and motif content of all the peptides in the array library. In some embodiments, the discriminating peptides of the immunosignature binding patterns that distinguish a subject that is known or suspected of having an AI from reference subjects that have a different disease can be enriched in at least one, at least two, at least three, at least four, at least five, at least six, at least seven, at least eight, at least nine, or at least ten different amino acids. In some embodiments, enrichment of the amino acids in discriminating peptides can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% relative to the total content of each of the amino acids present in all the library peptides.

Similarly, in some embodiments, the discriminating peptides of the immunosignature binding patterns that distinguish a subject that has an AI disease from reference subjects that have a different disease can be enriched in at least one, at least two, at least three, at least four, at least five, at least six, at least seven, at least eight, at least nine, or at least ten different sequence motifs. Enrichment of the sequence motifs can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% in at least one motif relative to the total content of each of the motifs present in all library peptides.

Enriched motifs were identified from the list of significant peptides unless that list was less than 100 peptides long, in which case the top 500 peptides based on the p-value associated with a Welch's t-test were used. The different n-mers in this list of peptides was compared to the same sized n-mers in the total library to determine if any were enriched. Fold enrichment is calculated by determining the number of times a motif (e.g. ABCD) occurs in the list divided by the number of times the motif (ABCD) occurs in the library. This value is further divided by the relative number of times the motif type (e.g., tetramers) appears in the library (i.e., total number of all tetramers in the list divided by the total number of tetramers in the library). The Enrichment (E) calculation can be represented by:

E=(m/M)/(t/T)

where m is the number of times the motif occurs as part of the discriminating peptide list; M is the total number of times the motif occurs in the library; t is the number of times the motif type appears in the list; and T is the number of times the motif occurs in the library. Fold enrichment can also be reported as Percent enrichment, i.e., “Enrichment value” multiplied by 100.

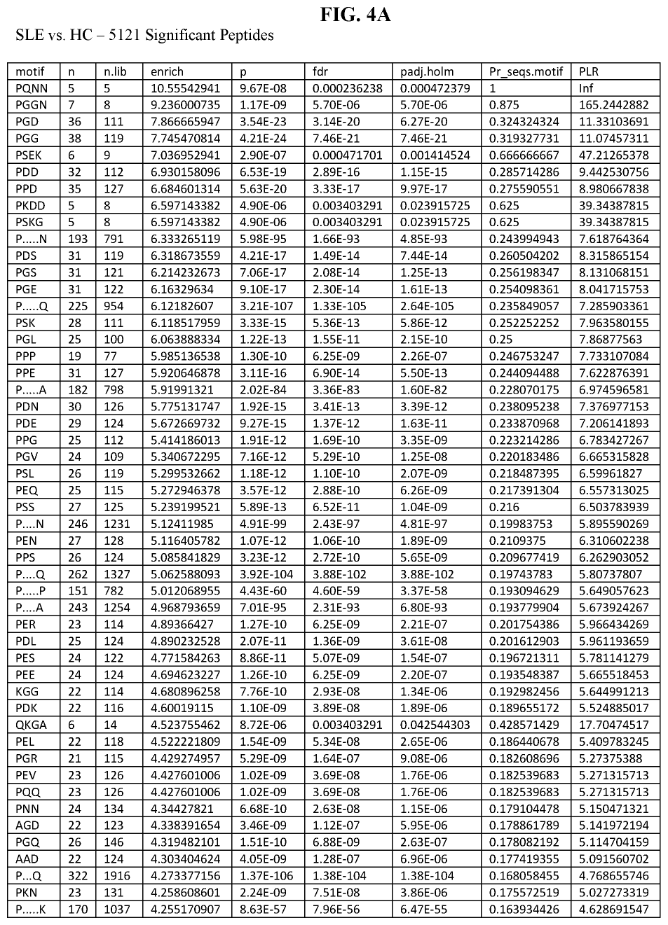

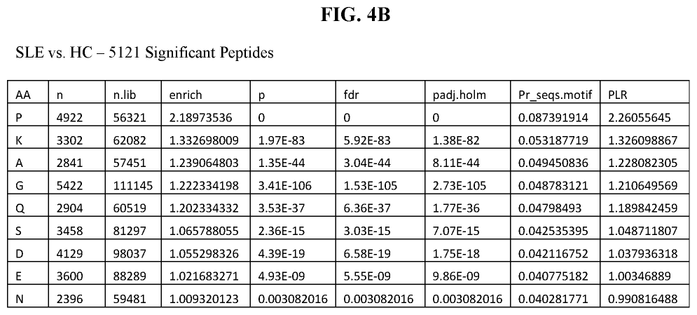

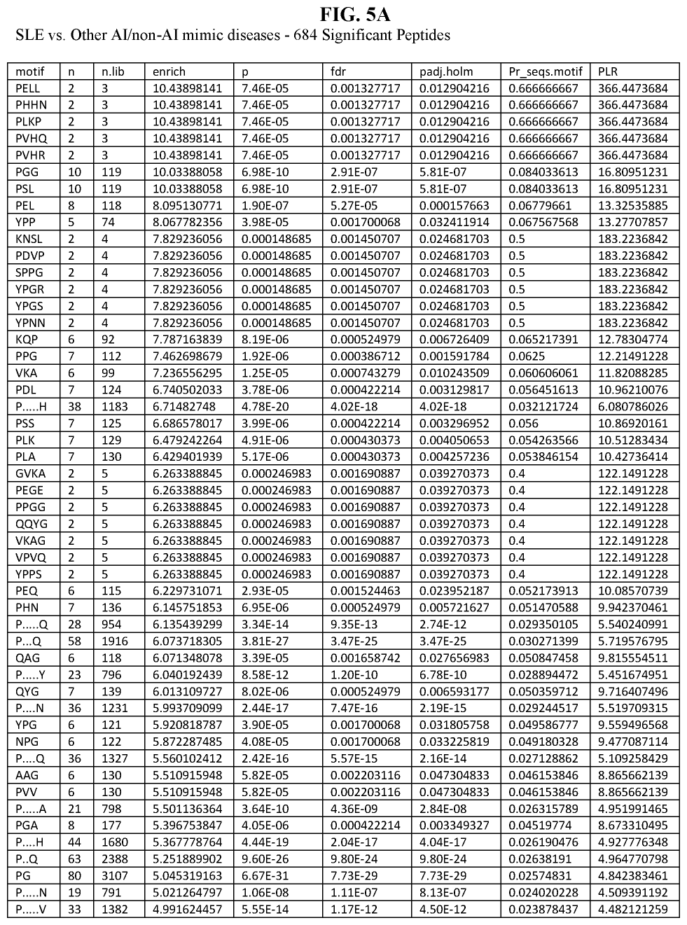

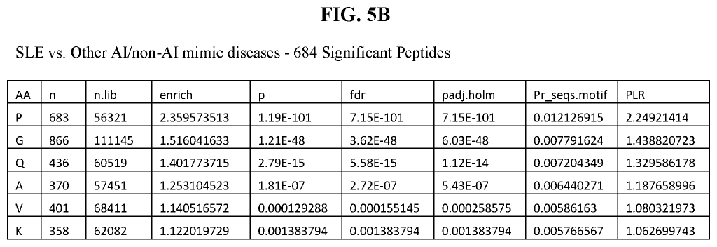

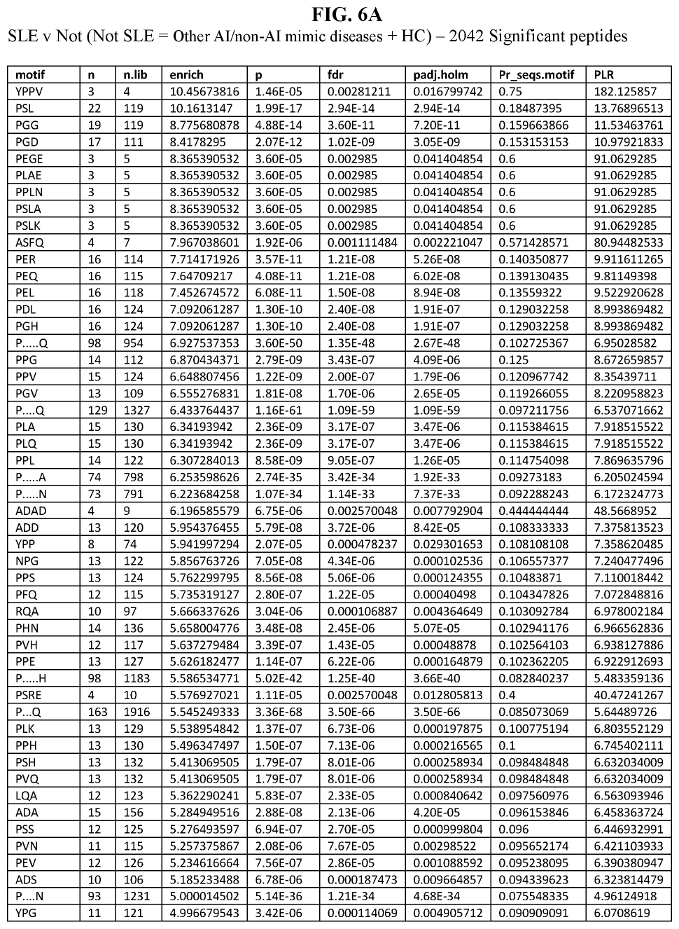

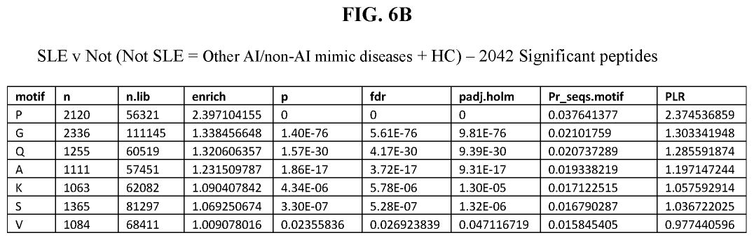

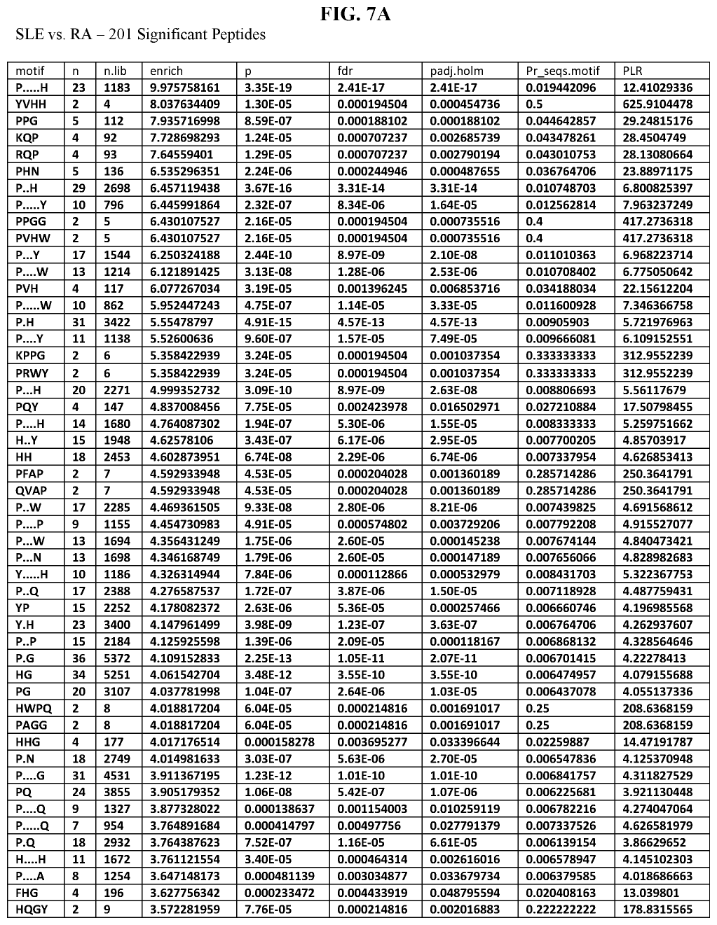

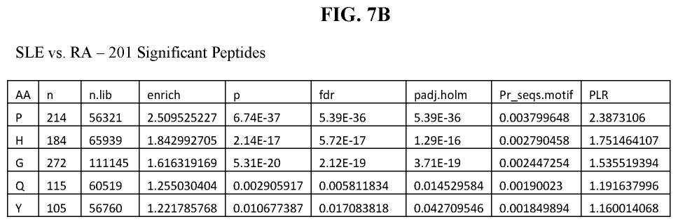

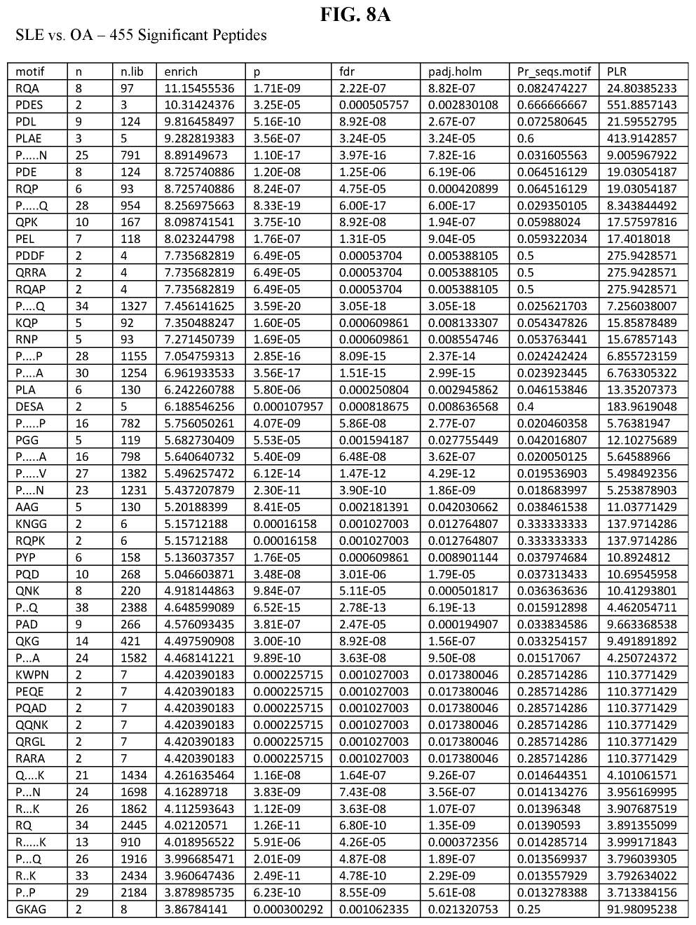

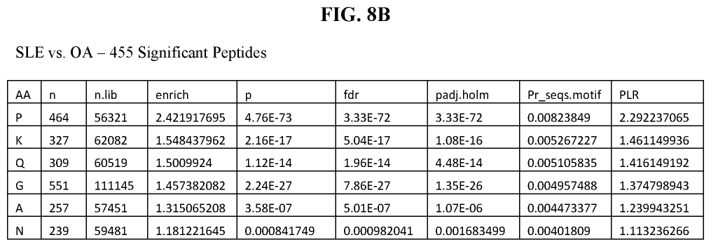

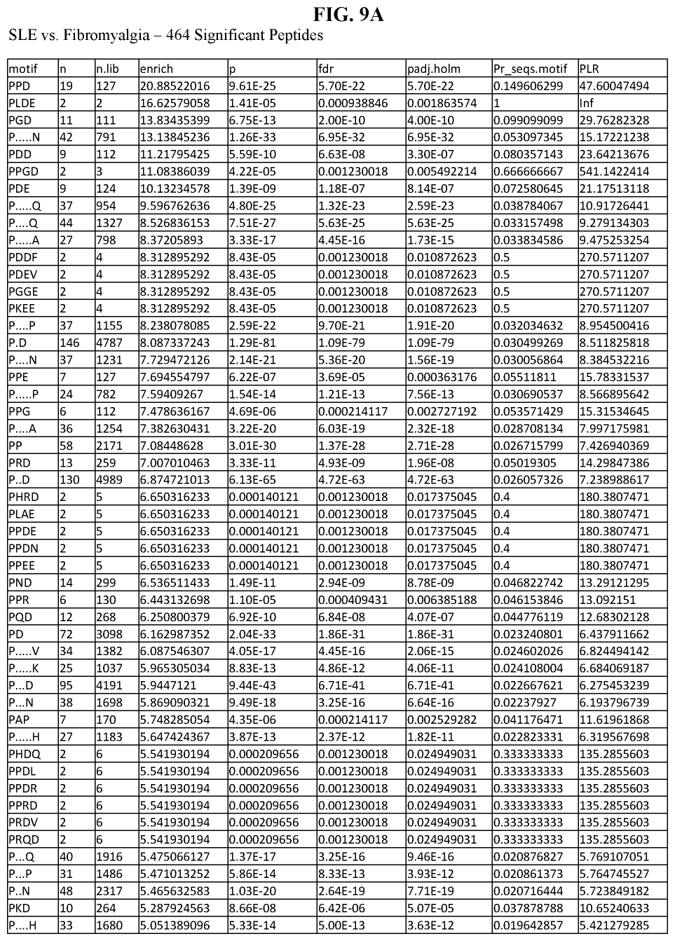

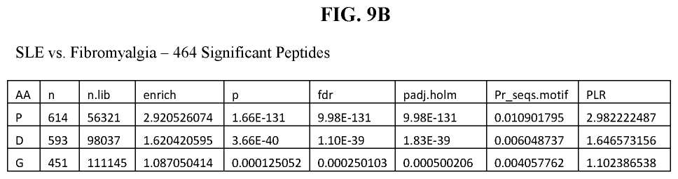

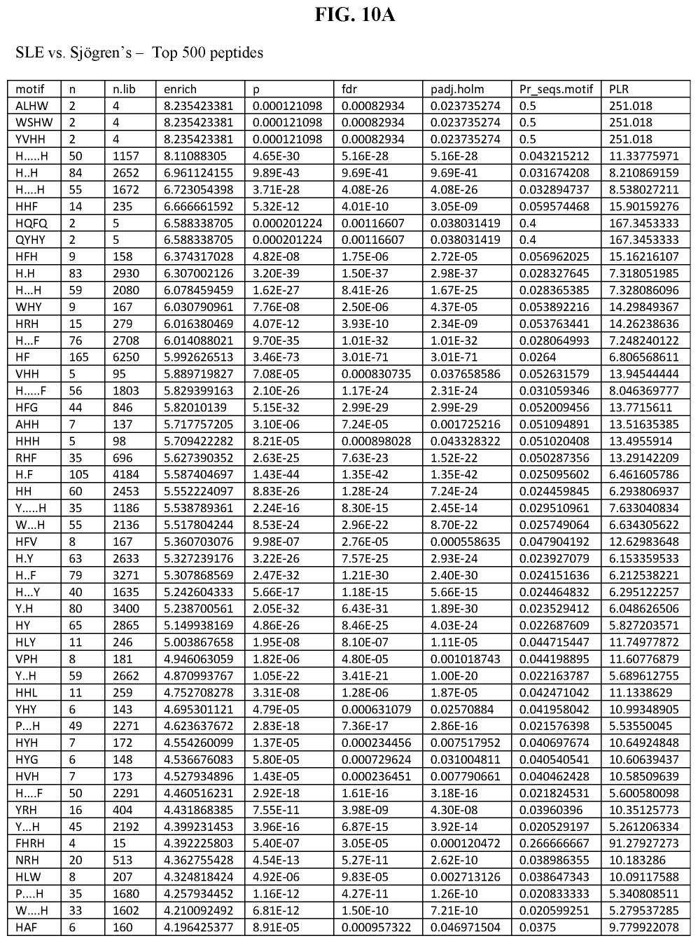

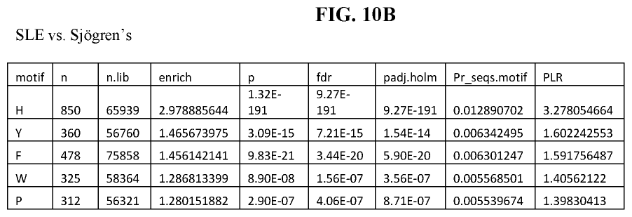

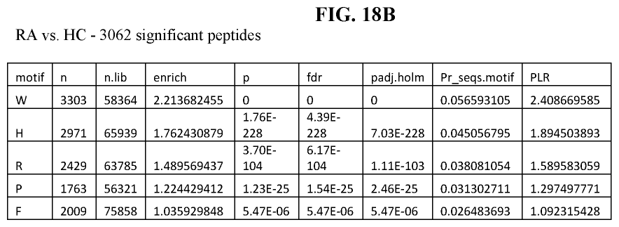

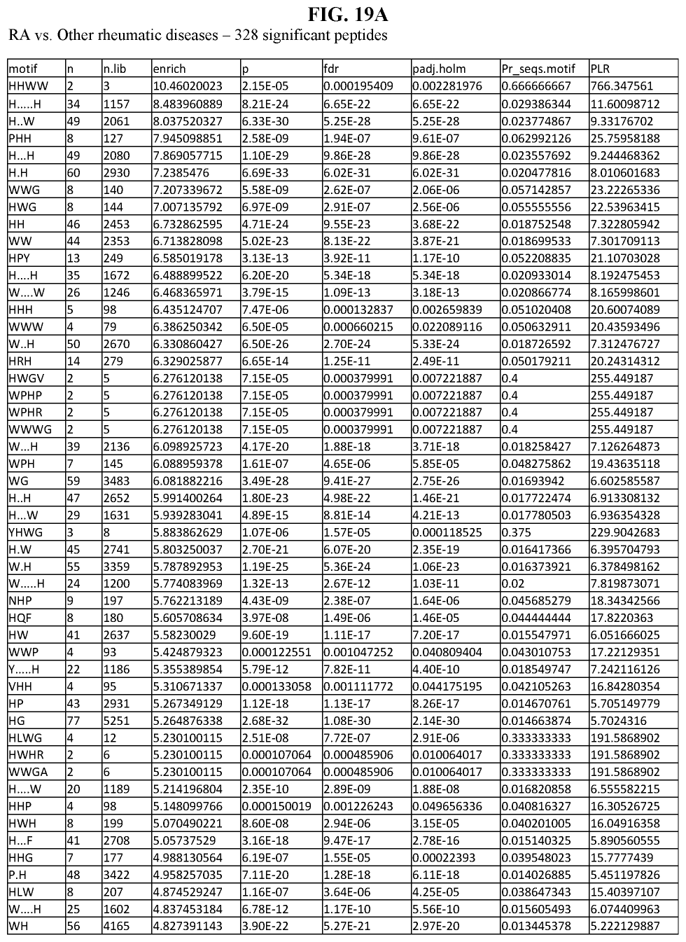

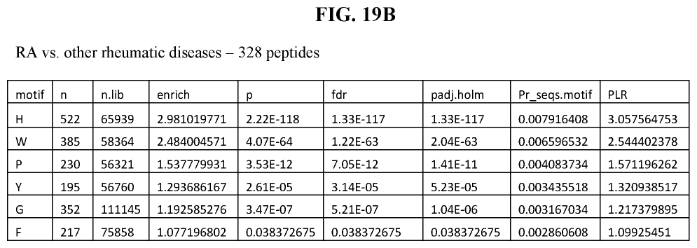

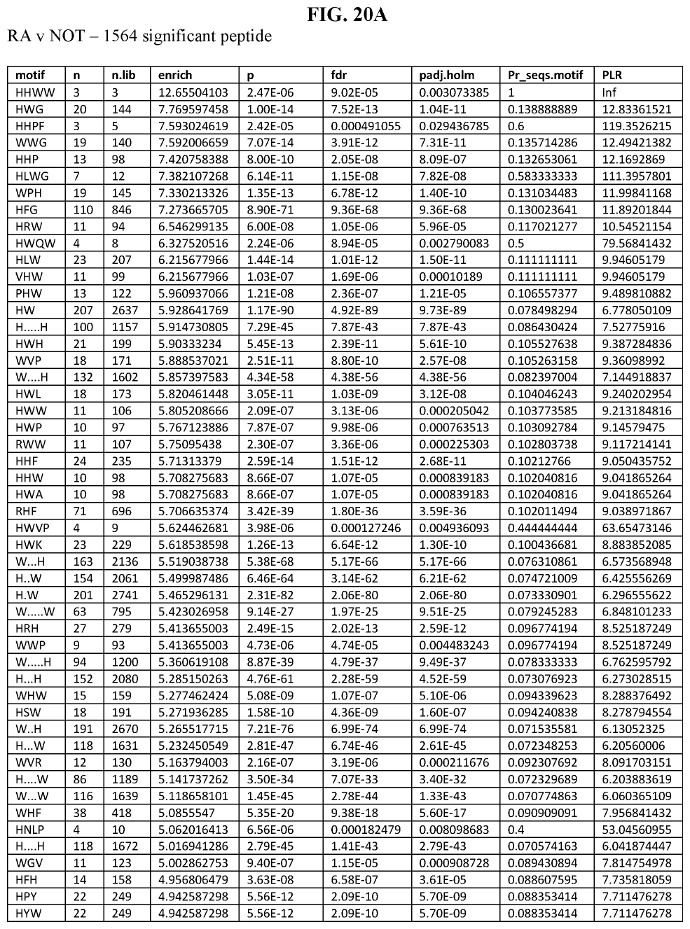

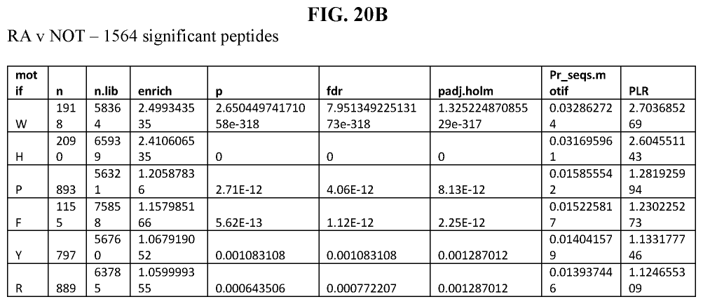

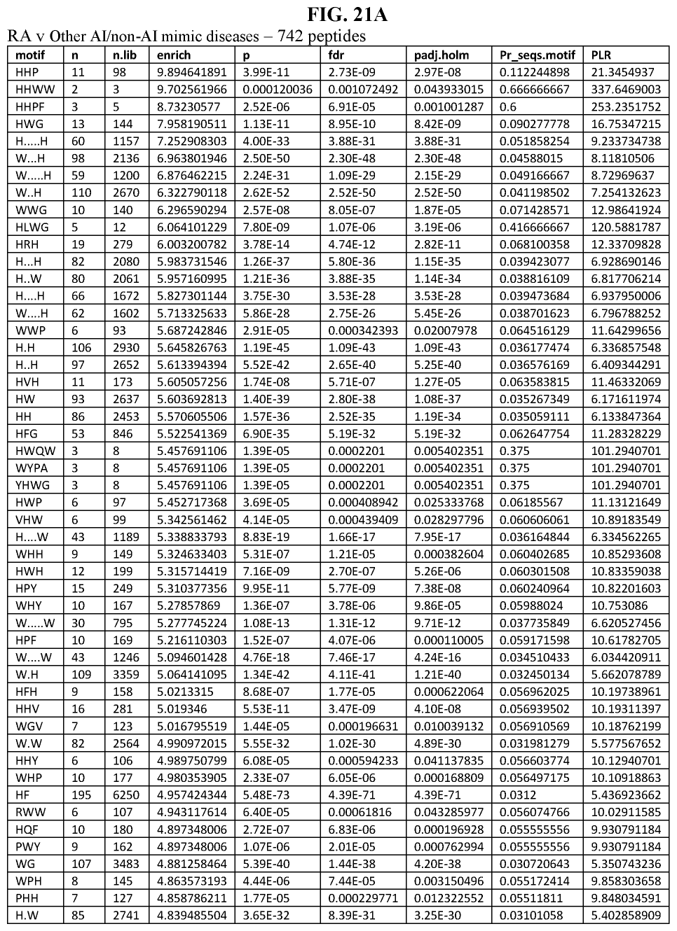

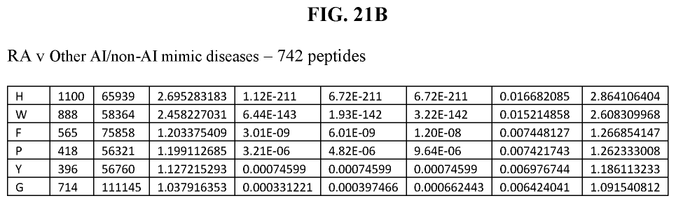

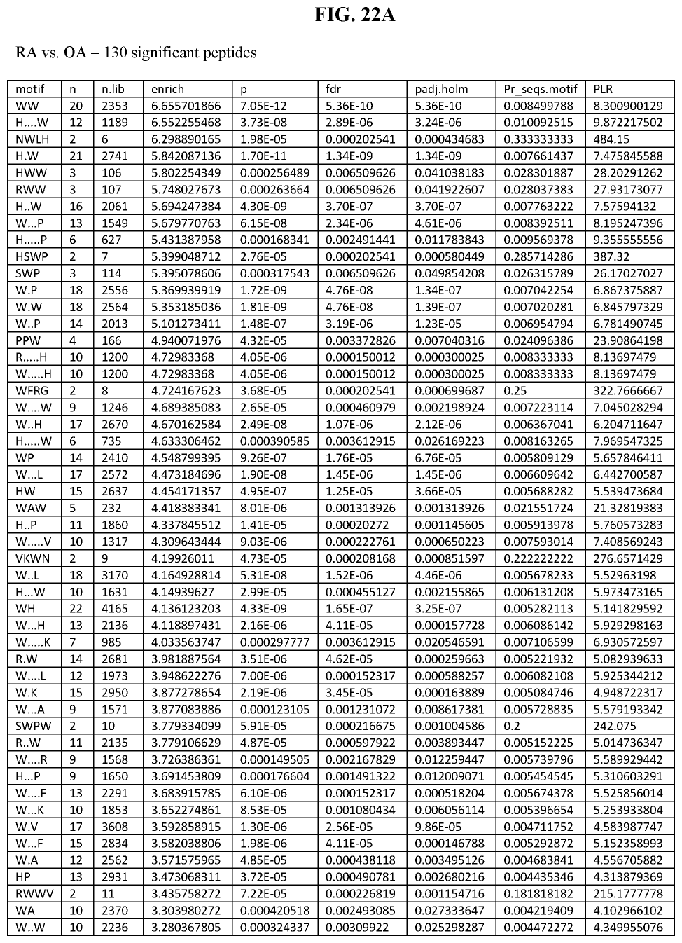

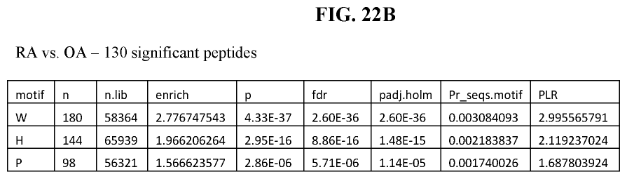

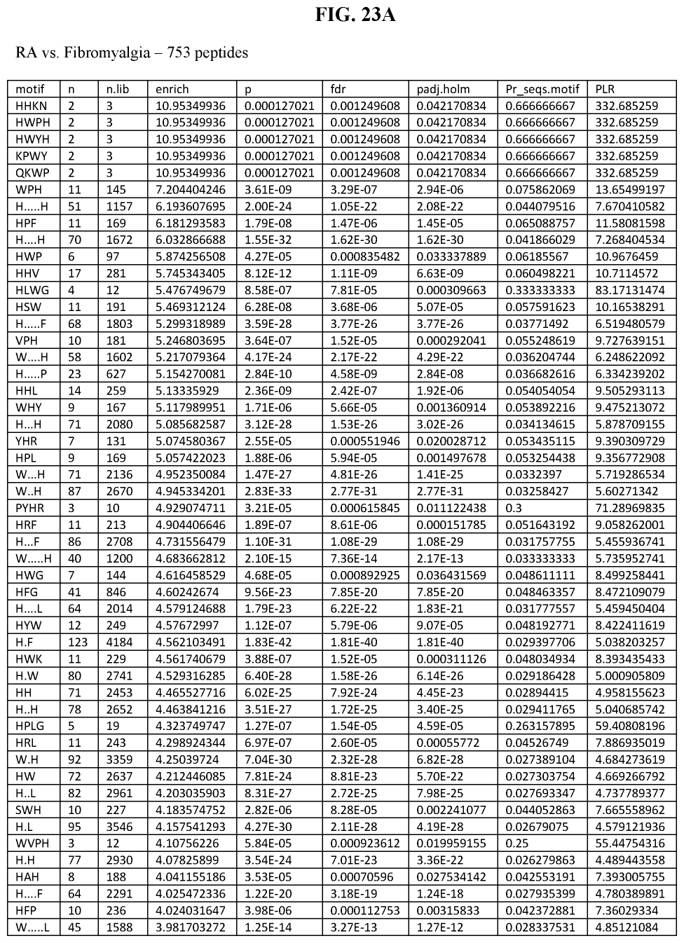

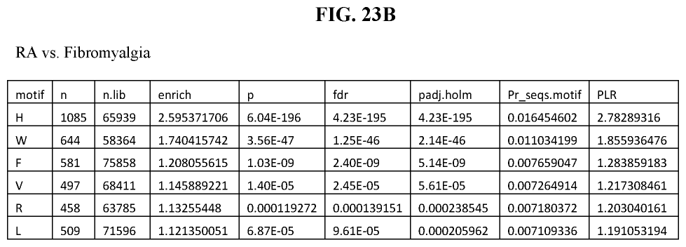

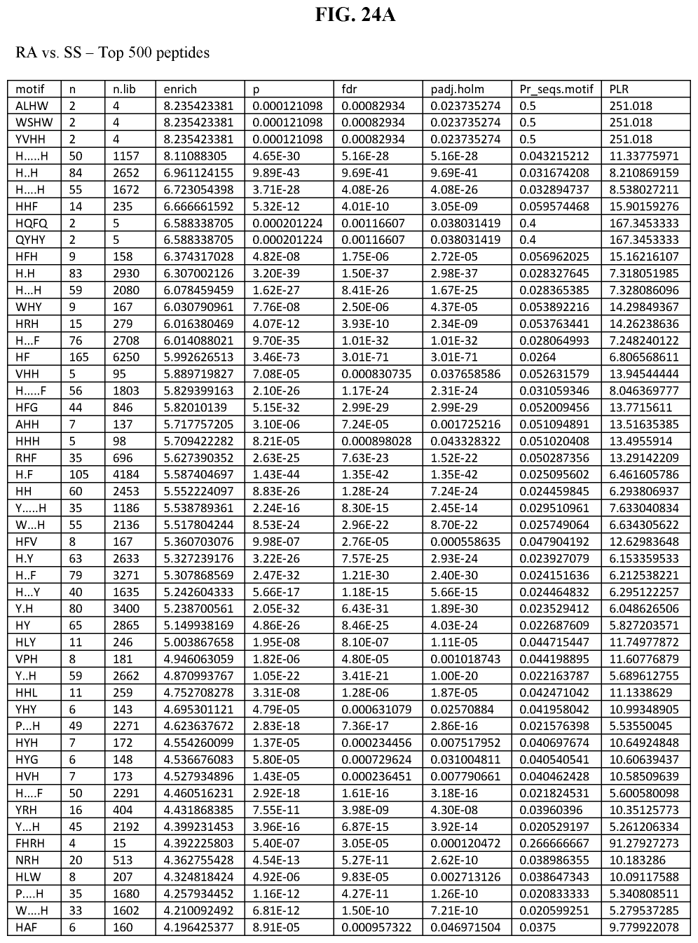

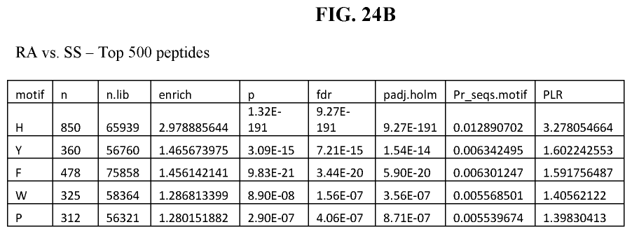

In some embodiments, the AI disease is SLE, and the discriminating peptides that distinguish samples from subjects with SLE from samples from subjects with conditions including a different autoimmune disease, non-auotimmune diseases, healthy subjects (HC), samples from a mixed population comprising subjects with other autoimmune disease and subjects with non-autoimmune mimic diseases, including and excluding healthy subjects in the mixed group, are enriched one or more of motifs as provided in FIG. 4A, FIG. 5A, FIG. 6A, FIG. 7A, FIG. 8A, FIG. 9A, and FIG. 10A. In other embodiments, the AI disease is RA, and the discriminating peptides that distinguish samples from subjects with RA from samples from subjects with conditions including a different autoimmune disease, non-auotimmune diseases, healthy subjects (HC), samples from a mixed population comprising subjects with other autoimmune disease and subjects with non-autoimmune mimic diseases, including and excluding healthy subjects in the mixed group, are enriched one or more of motifs as provided in FIG. 18A, FIG. 19A, FIG. 20A, FIG. 21A, FIG. 22A, FIG. 23A, and FIG. 24A. Enrichment of the one or more amino motifs can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total motif content of all the peptides in the array library.

In some embodiments, the AI disease is SLE, and the discriminating peptides that distinguish samples from subjects with SLE from samples from subjects with conditions including a different autoimmune disease, non-auotimmune diseases, healthy subjects (HC), samples from a mixed population comprising subjects with other autoimmune disease and subjects with non-autoimmune mimic diseases, including and excluding healthy subjects in the mixed group, are enriched in one or more amino acids FIG. 4B, FIG. 5B, FIG. 6B, FIG. 7B, FIG. 8B, FIG. 9B, and FIG. 10B. In other embodiments, the AI disease is RA, and the discriminating peptides that distinguish samples from subjects with RA from samples from subjects with conditions including a different autoimmune disease, non-auotimmune diseases, healthy subjects (HC), samples from a mixed population comprising subjects with other autoimmune disease and subjects with non-autoimmune mimic diseases, including and excluding healthy subjects in the mixed group, are enriched one or more amino acids as provided in FIG. 18B, FIG. 19B, FIG. 20B, FIG. 21B, FIG. 22B, FIG. 23B, and FIG. 24B. Enrichment of the one or more amino acids can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total amino acid content of all the peptides in the array library.

Enrichment of amino acid and motif content is relative to the corresponding total amino acid and motif content of all the peptides in the array library. In some embodiments, the discriminating peptides of the immunosignature binding patterns that distinguish a subject with an AI disease from a group of subjects having a different disease in differentially diagnosing or detecting the AI disease in a subject with the methods and arrays disclosed herein are enriched in at least one, at least two, at least three, at least four, at least five, at least six, at least seven, at least eight, at least nine, or at least ten different amino acids.

### Assay Performance

In some embodiments, the resulting method performance for differentially diagnosing an AI is characterized by an area under the Radio Operator Characteristic curve (ROC). Specificity, sensitivity, and accuracy metrics of the classification can be determined by the area under the ROC (AUC). The performance or accuracy of the method when applied to a plurality of patients whose health condition is already known by alternative methods may be characterized by an area under the receiver operator characteristic (ROC) curve (AUC) being greater than 0.90. In other embodiments, the method performance characterized by an area under the receiver operator characteristic (ROC) curve (AUC) being greater 0.70, greater than 0.80, greater than 0.90, greater than 0.95, method performance characterized by an area under the receiver operator characteristic (ROC) curve (AUC) being greater than 0.97, method performance characterized by an area under the receiver operator characteristic (ROC) curve (AUC) being greater than 0.99. In other embodiments, the method performance is characterized by an area under the receiver operator characteristic (ROC) curve (AUC) ranging from 0.60 to 0.69, 0.70 to 0.79, 0.80 to 0.89, or 0.90 to 1.0. In yet other embodiments, method performance is expressed in terms of sensitivity, specificity, and/or accuracy.

In some embodiments, the method has a sensitivity of at least 60%, for example 65%, 70%, 75%, 80%, 85%, 90%, 91%, 92%, 93%, 94%, 95%, 96%, 97%, 98%, 99% or 100% sensitivity.

In other embodiments, the method has a specificity of at least 60%, for example 65%, 70%, 75%, 80%, 85%, 90%, 91%, 92%, 93%, 94%, 95%, 96%, 97%, 98%, 99% or 100% specificity.

In some embodiments, the method has an accuracy of at least 60%, for example 65%, 70%, 75%, 80%, 85%, 90%, 91%, 92%, 93%, 94%, 95%, 96%, 97%, 98%, 99% or 100%.

### Identifying Candidate Biomarkers

The immunosignature obtained as provided can then be used in multiple applications comprising identifying candidate therapeutic targets, for differentially diagnosing an AI disease, monitoring the activity of the disease, and developing treatments for the individual against the identified AI disorder according to the methods and devices disclosed herein. In some embodiments, splaying the antibody repertoire out on an array of peptides (immunosignature, IS) and comparing samples from subjects with an AI disease e.g. SLE, to samples from subjects having a different disease e.g. RA, informative discriminating peptides can be identified to reveal the proteins recognized i.e. bound by the antibodies.

For example, the peptides can be identified with informatics methods. In cases where the informatics cannot identify a putative match, such as in the case of discontinuous epitopes, the informative peptide can be used as an affinity reagent to purify reactive antibody. Purified antibody can then be used in standard immunological techniques to identify the target.

Having differentially diagnosed an AI e.g. SLE, the appropriate reference proteome can be queried to relate the sequences of the discriminating peptides bound by the antibodies in a sample. In some embodiments, the proteome that can be queried using the identified discriminating peptides is the human proteome RefSeq release 84, corresponding to human genome build GrCh38 (https://www.ncbi.nlm.nih.gov/refseq/), compiled Mar. 10, 2016. Additionally, other compilations of proteins that can be queried include without limitation lists of disease-relevant proteins, lists of proteins containing known or unknown mutations (including single nucleotide polymorphisms, insertions, substitutions and deletions), lists of proteins consisting of known and unknown splice variants, or lists of peptides or proteins from a combinatorial library (including natural and unnatural amino acids).

Software for aligning single and multiple proteins to a proteome or protein list include without limitation BLAST, CS-BLAST, CUDAWS++, DIAMOND, FASTA, GGSEARCH (GG or GL), Genoogle, HMMER, H-suite, IDF, KLAST, MMseqs2, USEARCH, OSWALD, Parasail, PSI-BLAST, PSI Protein, Sequilab, SAM, SSEARCH, SWAPHI, SWIMM, and SWIPE.

Alternatively, sequence motifs that are enriched in the discriminating peptides relative to the motifs found in the entire peptide library on the array can be aligned to a proteome to identify target proteins that can be validated as possible therapeutic targets for the treatment of the condition. Online databases and search tools for identifying protein domains, families and functional sites are available e.g. Prosite at ExPASy, Motif Scan (MyHits, SIB, Switzerland), Interpro 5, MOTIF (GenomeNet, Japan), and Pfam (EMBL-EBI).

In some embodiments, the alignment method can be any method for mapping amino acids of a query sequence onto a longer protein sequence, including BLAST (Altschul, S.F. & Gish, W. [1996] “Local alignment statistics.” Meth. Enzymol. 266:460-480), the use of compositional substitution and scoring matrices, exact matching with and without gaps, epitope prediction, antigenicity prediction, hydrophobicity prediction, surface accessibility prediction. For each approach, a canonical or modified scoring system can be used, with the modified scoring system optimized to correct for biases in the peptide library composition. In some embodiments, a modified BLAST alignment is used, requiring a seed of 3 amino acids with a gap penalty of 4, with a scoring matrix of BLOSUM62 (Henikoff, J. G. Proc. Natl. Acad. Sci. USA 89, 10915-10919 [1992]) modified to reflect the amino acid composition of the array (States et al., Methods 3:66-70 [1991]). These modifications increase the score of similar substitutions, remove penalties for amino acids absent from the array and score all exact matches equally.

The discriminating peptides that can be used to identify candidate biomarker proteins according to the method provided, are chosen according to their ability to distinguish between two or more different health conditions. As described elsewhere herein, discriminating peptides can be chosen at a predetermined statistical stringency, e.g. by p-value, for the probability of discriminating between two or more conditions; by differences in the relative binding signal intensity changes between two or more conditions; by their intensity rank in a single condition; by their coefficients in a machine learning model trained against two or more conditions e.g. AUC, or by their correlation with one or more study parameters, e.g. R squared, Spearman correlation. In some embodiments, the discriminating peptides selected for identifying one or more candidate biomarkers are chosen as having a p-value of p<1E-03, p<1E-04, or p<1E-05.

Having identified the set of discriminating peptides for the differential diagnosis of an AI disease as described elsewhere herein, the discriminating peptides are aligned to one or more pathogen proteomes, and peptides having a positive BLAST score are identified. For each of the proteins to which discriminating peptides are aligned, the scores for the BLAST-positive peptides in the alignment are assembled into a matrix e.g. modified BLOSUM62, with each row of the matrix corresponding to an aligned peptide and each column corresponding to one of the consecutive amino acids that comprises the protein. Each row of the matrix corresponds to an aligned peptide and each column corresponds to an amino acid on the protein, with gaps and deletions allowed within the peptide rows to allow for alignment to the protein.

Using the modified BLAST scoring matrix described above, each position in the matrix receives the score for paired amino acids of the peptide and protein in that column. Then, for each amino acid in the protein, the corresponding column is summed to create an amino acid “overlap score” that represents coverage of that amino acid at a position in the protein by the discriminating peptides.

The amino acid overlap score is subsequently corrected for the composition i.e. the amino acid content of the array library. For example, a correction is made to account for library array peptides that exclude one or more of the 20 natural amino acids. To correct this score for library composition, an amino acid overlap score is calculated by the same method for a list of all array peptides. This allows for the calculation of a peptide overlap difference score based on the discriminating peptides, s_{d}, at each amino acid position according to the following equation:

s_{d}=a−(b/d)*c

where “a” is the overlap score from the discriminating peptides, “b” is the number of ImmunoSignature discriminating peptides, “c” is the overlap score for the full array of peptide and “d” is the number of library peptides on the entire array.

Next, the amino acid overlap score obtained from the alignment of the discriminating peptides is converted to a protein score, S_{d}. To convert the scores at the amino acid level, s_{d}, to a full-protein statistic, S_{d}, the sum of scores for every possible tiling n-mer epitope within a protein is calculated, and the final score is the maximum score obtained along this rolling window of n-mers for each protein, where n can be 20 (etc). In some embodiments, the scores can be obtained for tiling 10-mer epitopes, 15-mer-epitopes, 20-mer epitopes, 25-mer epitopes, 30 mer-epitopes, 35-mer-epitopes, 40-mer-epitopes, 45-mer epitopes, or 50-mer epitopes. Protein score S_{d} is the maximum score obtained along the rolling window. In some embodiments, the n-mer correlates to the entire length of the protein i.e. the discriminating peptides are aligned to the entire sequence of the protein. Alternatively, the scores can be obtained by aligning the peptide sequences to the entire protein sequences.

Ranking of the identified candidate biomarkers is made subsequently relative to the ranking of randomly chosen non-discriminating peptides. Accordingly, an overlap score for non-discriminating peptides (non-discriminating random ‘sᵣ’ score) i.e. randomly chosen peptides that align to each of one or more proteins of a same proteome or protein list is obtained as described for the discriminating peptides. The amino acid overlap score is calculated for the random peptides, and is subsequently corrected for amino acid content of the peptide library to provide a non-discriminating or random sᵣ score. The non-discriminating sᵣ score is then converted to a non-discriminating protein ‘Sᵣ’ score for each of a plurality of randomly chosen non-discriminating peptides. For example, non-discriminating random protein ‘Sᵣ’ scores can be obtained for at least 25, at least 50, at least 100, at least 150, at least 200, or more randomly-chosen non-discriminating peptides. In some embodiments, the final protein score, Sᵣ score-for the randomly chosen non-discriminating peptides can be calculated using the equivalent number of discriminating peptides used to obtain protein score S_{d}. In other embodiments, at least 20%, at least 30%, at least 40%, at least 50%, at least 60%, at least 70%, at least 80%, at least 85%, at least 90%, at least 95%, at least 98%, at least 99% of the number of discriminating peptides used to determine S_{d} are used to determine the non-discriminating protein ‘Sᵣ’ score.

In some embodiments, the candidate protein biomarkers are ranked by their S_{d} score relative to the Sᵣ score of the proteins identified by alignment of non-discriminating peptides. In some embodiments, ranking can be determined according to a p-value. Top candidate biomarkers can be chosen as having a p-value less than 10⁻³, less than 10⁻⁴, less than 10⁻⁵, less than 10⁻⁶, less than 10⁻⁷, less than 10⁻⁸, less than 10⁻⁹, less than 10⁻¹⁰, less than 10⁻¹², less than 10⁻¹⁵, less than 10⁻¹⁸, less than 10⁻²⁰, or less. In some embodiments, at least 5, at least 10, at least 15, at least 20, at least 30, at least 40, at least 50, at least 60, at least 70, at least 80, at least 90, at least 100, at least 120, at least 150, at least 180, at least 200, at least 250, at least 300, at least 350, at least 400, at least 450, at least 500, or more candidate biomarkers are identified according to the method.

In other embodiments, candidate biomarkers are chosen according to the S_{d} score obtained by tiling a plurality of discriminating peptides to n-mer epitopes as described in the preceding paragraphs, and selecting the number of candidate biomarkers as a percent of proteins having the greatest S_{d} score for the pathogen's proteome. In some embodiments, candidate biomarkers are proteins having the highest ranking S_{d} scores and comprising at least 0.01% of the total number of proteins of the pathogens' proteome. In other embodiments, candidate biomarkers are proteins having the highest ranking S_{d} scores and comprising at least 0.02%, at least 0.03%, at least 0.04%, at least 0.05%, at least 0.1%, at least 0.15%, at least 0.2%, at least 0.25%, at least 0.3%, at least 0.35%, at least 0.4%, at least 0.45%, at least 0.5%, at least 0.55%, at least 0.6%, at least 0.65%, at least 0.7%, at least 0.75%, at least 0.8%, at least 0.85%, at least 0.9%, at least 1%, at least 2%, at least 3%, at least 4%, at least 5%, at least 10%, at least 20%, or more of the total number of proteins of the pathogens' proteome.

In some embodiments, a method or kit is provided for identifying at least one candidate protein biomarker for systemic lupus erythematosus (SLE), the method comprising: (a) providing a peptide array and incubating a biological sample from a plurality of reference subjects known to have systemic lupus erythematosus to the peptide array; (b) identifying a set of discriminating peptides bound to antibodies in the biological sample from said subject, the set of discriminating peptides displaying binding signals capable of differentiating systemic lupus erythematosus from samples from healthy subjects; (c) querying a proteome database with each of the peptides in the set of discriminating peptides; (d) aligning each of the peptides in the set of discriminating peptides to one or more proteins in the human proteome database; and (e) obtaining a relevance score and ranking for each of the identified proteins from the proteome database; wherein each of the identified proteins is a candidate biomarker for systemic lupus erythematosus. The discriminating peptides can be identified by statistical means e.g. t-test, as having p-values of less than 10⁻³, less than 10⁻⁴, less than 10⁻⁵, less than 10⁻⁶, less than 10⁻⁷, less than 10⁻⁸, less than 10⁻⁹, less than 10⁻¹⁰, less than 10⁻¹¹, less than 10⁻¹², less than 10⁻¹³, less than 10⁻¹⁴, or less than 10⁻¹⁵. In some embodiments, the resulting candidate biomarkers can be ranked according to a p-value of less than less than 10⁻³, less than less than 10⁻⁴, less than less than 10⁻⁵, or less than less than 10⁻⁶ when compared to proteins identified according to the method but using non-discriminating peptides.

### Samples

The samples that are utilized according to the methods provided can be any biological samples. For example, the biological sample can be a biological liquid sample that comprises antibodies. Suitable biological liquid samples include, but are not limited to blood, plasma, serum, sweat, tears, sputum, urine, stool water, ear flow, lymph, saliva, cerebrospinal fluid, ravages, bone marrow suspension, vaginal flow, transcervical lavage, synovial fluid, aqueous humor, amniotic fluid, cerumen, breast milk, broncheoalveolar lavage fluid, brain fluid, cyst fluid, pleural and peritoneal fluid, pericardial fluid, ascites, milk, pancreatic juice, secretions of the respiratory, intestinal and genitourinary tracts, amniotic fluid, milk, and leukophoresis samples. A biological sample may also include the blastocyl cavity, umbilical cord blood, or maternal circulation which may be of fetal or maternal origin. In some embodiments, the sample is a sample that is easily obtainable by non-invasive procedures e.g. blood, plasma, serum, sweat, tears, sputum, urine, sputum, ear flow, or saliva. In certain embodiments the sample is a peripheral blood sample, or the plasma or serum fractions of a peripheral blood sample. As used herein, the terms “blood,” “plasma” and “serum” expressly encompass fractions or processed portions thereof.

Because of its minimally invasive accessibility and its ready availability, blood is the most preferred and used human body fluid to be measured in routine clinical practice. Moreover, blood perfuses all body tissues and its composition is therefore relevant as an indicator of the over-all physiology of an individual. In some embodiments, the biological sample that is used to obtain an immunosignature/antibody binding profile is a blood sample. In other embodiments, the biological sample is a plasma sample. In yet other embodiments, the biological sample is a serum sample. In yet other embodiments, the biological sample is a dried blood sample. The biological sample may be obtained through a third party, such as a party not performing the analysis of the antibody binding profiles, and/or the party performing the binding assay to the peptide array. For example, the sample may be obtained through a clinician, physician, or other health care manager of a subject from which the sample is derived. Alternatively, the biological sample may be obtained by the party performing the binding assay of the sample to a peptide array, and/or the same party analyzing the antibody binding profile/IS. Biological samples that are to be assayed, can be archived (e.g., frozen) or otherwise stored in under preservative conditions.

The terms “patient sample” and “subject sample” are used interchangeably herein to refer to a sample e.g. a biological sample, obtained from a patient i.e. a recipient of medical attention, care or treatment. The subject sample can be any of the samples described herein. In certain embodiments, the subject sample is obtained by non-invasive procedures e.g. peripheral blood sample.

An antibody binding profile of circulating antibodies in a sample can be obtained according to the methods provided using limited quantities of sample. For example, peptides on the array can be contacted with a fraction of a milliliter of blood to obtain an antibody binding profile comprising a sufficient number of informative peptide-protein complexes to identify the health condition of the subject.

In some embodiments, the volume of biological sample that is needed to obtain an antibody binding profile is less than 10 ml, less than 5 ml, less than 3 ml, less than 2 ml, less than 1 ml, less than 900 ul, less than 800 ul, less than 700 ul, less than 600 ul, less than 500 ul, less than 400 ul, less than 300 ul, less than 200 ul, less than 100 ul, less than 50 ul, less than 40 ul, less than 30 ul, less than 20 ul, less than 10 ul, less than 1 ul, less than 900 nl, less than 800 nl, less than 700 nl, less than 600 nl, less than 500 nl, less than 400 nl, less than 300 nl, less than 200 nl, less than 100 nl, less than 50 nl, less than 40 nl, less than 30 nl, less than 20 nl, less than 10 nl, or less than 1 nl. In some embodiments, the biological fluid sample can be diluted several fold to obtain a antibody binding profile. For example, a biological sample obtained from a subject can be diluted at least by 2-fold, at least by 4-fold, at least by 8-fold, at least by 10-fold, at least by 15-fold, at least by 20-fold, at least by 30-fold, at least by 40-fold, at least by 50-fold, at least by 100-fold, at least by 200-fold, at least by 300-fold, at least by 400-fold, at least by 500-fold, at least by 600-fold, at least by 700-fold, at least by 800-fold, at least by 900-fold, at least by 1000-fold, at least by 5000-fold, or at least by 10,000-fold. Antibodies present in the diluted serum sample, and are considered significant to the health of the subject, because if antibodies remain present even in the diluted serum sample, they must reasonably have been present at relatively high amounts in the blood of the patient.

### Treatments and Conditions

The methods and arrays of the invention provide methods, assays and devices for identifying discriminating peptides, which can be used for differentially diagnosing AI diseases, and identifying candidate biomarkers of the Ai diseases. The methods and arrays of the embodiments disclosed herein can be used, for example, for differential diagnosis of an AI and/or identifying one or more candidate biomarkers for the AI in a subject. A subject can be a human, a guinea pig, a dog, a cat, a horse, a mouse, a rabbit, and various other animals. A subject can be of any age, for example, a subject can be an infant, a toddler, a child, a pre-adolescent, an adolescent, an adult, or an elderly individual.

The arrays and methods of the invention can be used by a user. A plurality of users can use a method of the invention to identify and/or provide a treatment of a condition. A user can be, for example, a human who wishes to monitor one's own health. A user can be, for example, a health care provider. A health care provider can be, for example, a physician. In some embodiments, the user is a health care provider attending the subject. Non-limiting examples of physicians and health care providers that can be users of the invention can include, an anesthesiologist, a bariatric surgery specialist, a blood banking transfusion medicine specialist, a cardiac electrophysiologist, a cardiac surgeon, a cardiologist, a certified nursing assistant, a clinical cardiac electrophysiology specialist, a clinical neurophysiology specialist, a clinical nurse specialist, a colorectal surgeon, a critical care medicine specialist, a critical care surgery specialist, a dental hygienist, a dentist, a dermatologist, an emergency medical technician, an emergency medicine physician, a gastrointestinal surgeon, a hematologist, a hospice care and palliative medicine specialist, a homeopathic specialist, an infectious disease specialist, an internist, a maxillofacial surgeon, a medical assistant, a medical examiner, a medical geneticist, a medical oncologist, a midwife, a neonatal-perinatal specialist, a nephrologist, a neurologist, a neurosurgeon, a nuclear medicine specialist, a nurse, a nurse practioner, an obstetrician, an oncologist, an oral surgeon, an orthodontist, an orthopedic specialist, a pain management specialist, a pathologist, a pediatrician, a perfusionist, a periodontist, a plastic surgeon, a podiatrist, a proctologist, a prosthetic specialist, a psychiatrist, a pulmonologist, a radiologist, a surgeon, a thoracic specialist, a transplant specialist, a vascular specialist, a vascular surgeon, and a veterinarian. A diagnosis identified with an array and a method of the invention can be incorporated into a subject's medical record.

### Array Platform

In some embodiments, disclosed herein are methods, processes, and kits that provide for array platforms that allow for increased diversity and fidelity of chemical library synthesis. The array platforms comprise a plurality of individual features on the surface of the array. Each feature typically comprises a plurality of individual molecules i.e. peptides, which are optionally synthesized in situ on the surface of the array, wherein the molecules are identical within a feature, but the sequence or identity of the molecules differ between features. The array molecules include, but are not limited to nucleic acids (including DNA, RNA, nucleosides, nucleotides, structure analogs or combinations thereof), peptides, peptide-mimetics, and combinations thereof and the like, wherein the array molecules may comprise natural or non-natural monomers within the molecules. Such array molecules include the synthesis of large synthetic peptide arrays. In some embodiments, a molecule in an array is a mimotope, a molecule that mimics the structure of an epitope and is able to bind an epitope-elicited antibody. In some embodiments, a molecule in the array is a paratope or a paratope mimetic, comprising a site in the variable region of an antibody (or T cell receptor) that binds to an epitope an antigen. In some embodiments, an array of the invention is a peptide array comprising random, pseudo-random or maximally diverse peptide sequences.

The peptide arrays can include control sequences that match epitopes of well characterized monoclonal antibodies (mAbs). Binding patterns to control sequences and to library peptides can be measured to qualify the arrays and the immunosignaturing assay process. mAbs with known epitopes e.g. 4C1, p53Ab1, p53Ab8 and LnKB2, can be assayed at different doses. Additionally, inter wafer signal precision can be determined by testing sample replicates e.g. plasma samples, on arrays from different wafers and calculating the coefficients of variation (CV) for all library peptides. Precision of the measurements of binding signals can be determined as an aggregate of the inter-array, inter-slide, inter-wafer and inter-day variations made on arrays synthesized on wafers of the same batch (within wafer batches). Additionally, precision of measurements can be determined for arrays on wafers of different batches (between wafer batches). In some embodiments, measurements of binding signals can be made within and/or between wafer batches with a precision varying less than 5%, less than 10%, less than 15%, less than 20%, less than 25%, or less than 30%.

The technologies disclosed herein include a photolithographic array synthesis platform that merges semiconductor manufacturing processes and combinatorial chemical synthesis to produce array-based libraries on silicon wafers. By utilizing the tremendous advancements in photolithographic feature patterning, the array synthesis platform is highly-scalable and capable of producing combinatorial chemical libraries with 40 million features on an 8-inch wafer. Photolithographic array synthesis is performed using semiconductor wafer production equipment in a class 10,000 cleanroom to achieve high reproducibility. When the wafer is diced into standard microscope slide dimensions, each slide contains more than 3 million distinct chemical entities.

In some embodiments, arrays with chemical libraries produced by photolithographic technologies disclosed herein are used for immune-based diagnostic assays, for example called immunosignature assays. Using a patient's antibody repertoire from a drop of blood bound to the arrays, a fluorescence binding profile image of the bound array provides sufficient information to differentially diagnose an AI disease.

In some embodiments, immunosignature assays are being developed for clinical application to differentially diagnose/monitor AI diseases and to assess response to treatments. Exemplary embodiments of immunosignature assays is described in detail in US Pre-Grant Publication No. 2012/0190574, entitled “Compound Arrays for Sample Profiling” and US Pre-Grant Publication No. 2014/0087963, entitled “Immunosignaturing: A Path to Early Diagnosis and Health Monitoring”, both of which are incorporated by reference herein for such disclosure. The arrays developed herein incorporate analytical measurement capability within each synthesized array using orthogonal analytical methods including ellipsometry, mass spectrometry and fluorescence. These measurements enable longitudinal qualitative and quantitative assessment of array synthesis performance.

In some embodiments, the array is a wafer-based, photolithographic, in situ peptide array produced using reusable masks and automation to obtain arrays of scalable numbers of combinatorial sequence peptides. In some embodiments, the peptide array comprises at least 5,000, at least 10,000, at least 15,000, at least 20,000, at least 30,000, at least 40,000, at least 50,000, at least 100,000, at least 200,000, at least 300,000, at least 400,000, at least 500,000, at least 1,000,000, at least 2,000,000, at least 3,000,000, at least 4,000,000, at least 5,000,000, at least 10,000,000, at least 100,000,000 or more peptides having different sequences. Multiple copies of each of the different sequence peptides can be situated on the wafer at addressable locations known as features.

In some embodiments, detection of antibody binding on a peptide array poses some challenges that can be addressed by the technologies disclosed herein. Accordingly, in some embodiments, the arrays and methods disclosed herein utilize specific coatings and functional group densities on the surface of the array that can tune the desired properties necessary for performing immunosignature assays. For example, non-specific antibody binding on a peptide array may be minimized by coating the silicon surface with a moderately hydrophilic monolayer polyethylene glycol (PEG), polyvinyl alcohol, carboxymethyl dextran, and combinations thereof. In some embodiments, the hydrophilic monolayer is homogeneous. Second, synthesized peptides are linked to the silicon surface using a spacer that moves the peptide away from the surface so that the peptide is presented to the antibody in an unhindered orientation.

The in situ synthesized peptide libraries are disease agnostic and can be synthesized without a priori awareness of a disease they are intended to diagnose. Identical arrays can be used to determine any health condition.

The term “peptide” as used herein refers to a plurality of amino acids joined together in a linear or circular chain. For purposes of the present invention, the term peptide is not limited to any particular number of amino acids. Preferably, however, they contain up to about 400 amino acids, up to about 300 amino acids, up to about 250 amino acids, up to about 150 amino acids, up to about 70 amino acids, up to about 50 amino acids, up to about 40 amino acids, up to 30 amino acids, up to 20 amino acids, up to 15 amino acids, up to 10 amino acids, or up to 5 amino acids. In some embodiments, the peptides of the array are between 5 and 30 amino acids, between 5 and 20 amino acids, or between 5 and 15 amino acids. The amino acids forming all or a part of a peptide molecule may be any of the twenty conventional, naturally occurring amino acids, i.e., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), and tyrosine (Y). Any of the amino acids in the peptides forming the present arrays may be replaced by a non-conventional amino acid. In general, conservative replacements are preferred. In some embodiments, the peptides on the array are synthesized from less of the 20 amino acids. In some embodiments, one or more of amino acids methionine, cysteine, isoleucine and threonine are excluded during synthesis of the peptides.

### Detector Device

In some embodiments, the systems, platforms and methods disclosed herein include a detector device for detecting binding on the array formats disclosed herein, including antibody binding on the peptide arrays disclosed herein. In some embodiments, used in conjunction with optical detection methods (ccd, pmt, other optical detector, optical filters and other optical detection deivces), detection of antibody binding is reported via optical detection in real-time or on a timed interval. In certain instances, quantification of final binding activity is reported via optical detection converted to AFU (arbitrary fluorescence units) or translated to electrical signal via impedance measurement or other electrochemical sensing. In other instances, antibody binding is detected by an emission or absorption of light or electromagnetic energy, either in the visible range or otherwise from an optically-detectable label on a probe applied to the peptide device. Optically detectable labels include, without limitation, fluorescent, chemiluminescent, electrochemiluminescent, luminescent, phosphorescent, fluorescence polarization, and charge labels. In some instances, a fluorescently labeled probe is active only in the presence of a specific target or antibody so that a fluorescent response from a sample signifies the presence of the target or antibody.

In some instances, light delivery schemes are utilized to provide the optical excitation and/or emission and/or detection of antibody binding. In certain embodiments, this includes using the flow cell materials (thermal polymers like acrylic (PMMA) cyclic olefin polymer (COP), cyclic olefin co-polymer, (COC), etc.) as optical wave guides to remove the need to use external components. In addition, in some instances light sources—light emitting diodes—LEDs, vertical-cavity surface-emitting lasers—VCSELs, and other lighting schemes are integrated directly inside the cartridge or detection device or built directly onto the peptide array surface to have internally controlled and powered light sources. PMTs, CCDs, or CMOS detectors can also be built into the detection device or cartridge.

### Digital Processing Device

In some embodiments, the systems, platforms, software, networks, and methods described herein include a digital processing device, or use of the same. In further embodiments, the digital processing device includes one or more hardware central processing units (CPUs), i.e., processors that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet.

In other embodiments, the digital processing device is optionally connected to a data storage device. In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, a digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, a digital processing device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, a digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, a digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera to capture motion or visual input. In still further embodiments, the input device is a combination of devices such as those disclosed herein.

In some embodiments, a digital processing device includes a digital camera. In some embodiments, a digital camera captures digital images. In some embodiments, the digital camera is an autofocus camera. In some embodiments, a digital camera is a charge-coupled device (CCD) camera. In further embodiments, a digital camera is a CCD video camera. In other embodiments, a digital camera is a complementary metal-oxide-semiconductor (CMOS) camera. In some embodiments, a digital camera captures still images. In other embodiments, a digital camera captures video images. In various embodiments, suitable digital cameras include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and higher megapixel cameras, including increments therein. In some embodiments, a digital camera is a standard definition camera. In other embodiments, a digital camera is an HD video camera. In further embodiments, an HD video camera captures images with at least about 1280×about 720 pixels or at least about 1920×about 1080 pixels. In some embodiments, a digital camera captures color digital images. In other embodiments, a digital camera captures grayscale digital images. In various embodiments, digital images are stored in any suitable digital image format. Suitable digital image formats include, by way of non-limiting examples, Joint Photographic Experts Group (JPEG), JPEG 2000, Exchangeable image file format (Exif), Tagged Image File Format (TIFF), RAW, Portable Network Graphics (PNG), Graphics Interchange Format (GIF), Windows® bitmap (BMP), portable pixmap (PPM), portable graymap (PGM), portable bitmap file format (PBM), and WebP. In various embodiments, digital images are stored in any suitable digital video format. Suitable digital video formats include, by way of non-limiting examples, AVI, MPEG, Apple® QuickTime®, MP4, AVCHD®, Windows Media®, DivX™, Flash Video, Ogg Theora, WebM, and RealMedia.

### Non-Transitory Computer Readable Storage Medium

In some embodiments, the systems, platforms, software, networks, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the systems, platforms, software, networks, and methods disclosed herein include at least one computer program. A computer program includes a sequence of instructions, executable in the digital processing device's CPU, written to perform a specified task. In light of the disclosure provided herein, those of skill in the art will recognize that a computer program may be written in various versions of various languages. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR).

In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™ JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. A web application for providing a career development network for artists that allows artists to upload information and media files, in some embodiments, includes a media player element. In various further embodiments, a media player element utilizes one or more of many suitable multimedia technologies including, by way of non-limiting examples, Adobe® Flash®, HTML 5, Apple® QuickTime®, Microsoft Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

The systems, platforms, software, networks, and methods disclosed herein include, in various embodiments, software, server, and database modules. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

The present invention is described in further detail in the following Examples which are not in any way intended to limit the scope of the invention as claimed. The attached Figures are meant to be considered as integral parts of the specification and description of the invention. The following examples are offered to illustrate, but not to limit the claimed invention.

### EXAMPLES

### Example 1—Immunosignature Methods

Immunosignature assays were used to differentiate autoimmune diseases (AI): Systemic Lupus Erythematosus (SLE) and Rheumatoid Arthritis (RA) from other autoimmune and mimic diseases including Osteoarthritis (OA), Sjogrens' disease (SS), Fibromyalgia (FM).

Donor Samples.

Donor plasma samples were obtained from the Albert Einstein College of Medicine (Bronx, N.Y.). A well-annotated cohort of 400 serum samples was prospectively collected for this study and included SLE (n=75), RA (n=95), Sjögren's (SS) (n=20), Osteo Arthritis (OA) (n=24), Fibromyalgia (n=22), other disease (OD) (n=76), “All Diseases” (AD) (n=237); “Other Rheumatic Diseases” (ORD) (n=144), and healthy controls (HC) (n=59).

Other Autoimmune Diseases and non-autoimmune mimic diseases (OD or Other AI) (n=76): ANCA Vasculitis (2), CIA (4), CNS Vasculitis, Dermatomyositis (6), Discoid Lupus, DMPM (3), DMPM/MCTD, GCA (2), Gout (9), Lupus (4), MCTD (9), Myositis (5), Overlap, Polyarticular Gout, Polychondritis, Polymyositis, Pseudogout, Psoriatic Arthritis (11), Scleroderma (7), Serospon (2), and Vasculitis (4). For SLE, the Other AI+non-AI mimic diseases further include fibromyalgia/RA, lupus/RA, OA/RA/serspon, RA/serspon, RA, and RAVASC. For RA, the Other AI+non-AI mimic diseases further include fibromyalgia/SLE, MCTD/SLE, SLE/MCTD, SLE/scleroderma, and SLE/SS.

“Other Rheumatic Diseases” (ORD) (n=144): SLE, SS, OA, psoriatic arthritis (11), gout (9), seronegative spondlyloarthropathy (2), pseudogout (1). Subjects with rheumatological diseases were diagnosed based on ACR criteria.

The “Not” group for SLE are samples of Other AI+non-AI mimic diseases+HC i.e. AI diseases other than SLE plus HC.

The “Not” group for RA are samples of Other AI+non-AI mimic diseases+HC i.e. AI diseases other than RA plus HC.

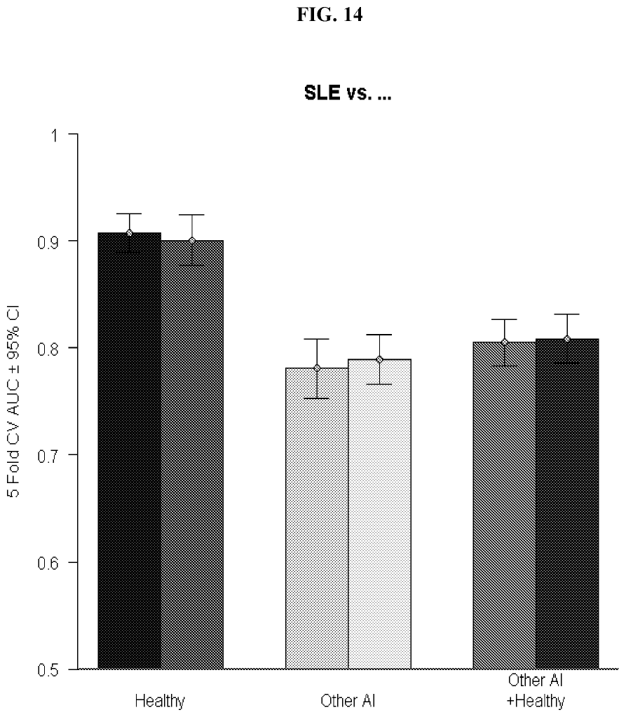

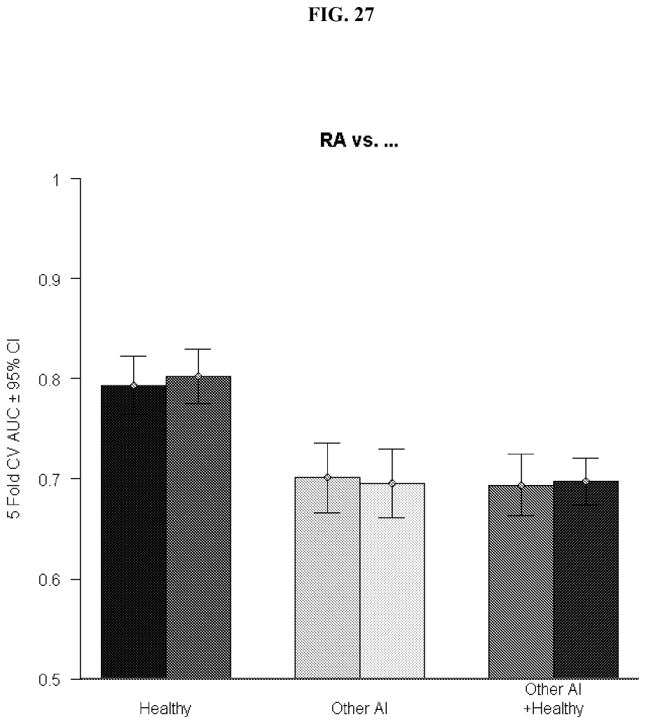

The “Mixed SLE and Other AI” and the “Mixed RA and Other AI” group indicated in FIG. 14 and FIG. 27, respectively represent a combination os samples from subjects with a mixed diagnosis and samples from subjects with other AI and/or mimic diseases: CIA/OA, gout/OA, OA/RA, OA/RA, OA/RA/serospon/DMPM/FM/SLE/scleroderma/DMPM/SLE, lupus/RA/MCTD/SLE, FM/lupus, FM/OA, FM/RA, FM/SLE,RA/serospon, RA/SLE, RA/SS, RA/vasc., SLE/MCTD, SLE/RA, SLE/scleroderma, SLE/SS, ANCA vasculitis, CIA, CNS vasculitis, dermatomyositis, Discoid lupus, DMPM, DMPM/MCTD, GCA, gout, lupus, MCTD, myositis, overlap, polyarticular gout, polychondritis, polymyositis, pseudogout, psoriatic arthritis, scleroderma, serospon, and vasculitis.

Samples were mixed 1:1 with ethylene glycol as a cryoprotectant and aliquoted into single use volumes. Single use aliquots were stored at −20° C. until needed. In each case, the remaining sample volume was stored neat at −80° C. Identities of all samples were tracked using 2D barcoded tubes (Micronic, Leystad, the Netherlands). In preparation for assay, sample aliquots were warmed on ice to 4° C. and diluted 1:100 in primary incubation buffer (Phosphate Buffered Saline with 0.05% Tween 20 (PBST) and 1% mannitol). Microtiter plates containing the 1:100 dilutions were then diluted to 1:625 for use in the assay.

Arrays.

A combinatorial library of 126,009 peptides with a median length of 9 residues and range from 5 to 13 amino acids was designed to include 99.9% of all possible 4-mers and 48.3% of all possible 5-mers of 16 amino acids (methionine, M; cysteine, C; isoleucine, I; and threonine, T were excluded). These were synthesized on an 200 mm silicon oxide wafer using standard semiconductor photolithography tools adapted for tert-butyloxycarbonyl (BOC) protecting group peptide chemistry (Legutki J B et al., Nature Communications. 2014; 5:4785). Briefly, an aminosilane functionalized wafer was coated with BOC-glycine. Next, photoresist containing a photoacid generator, which is activated by UV light, was applied to the wafer by spin coating. Exposure of the wafer to UV light (365 nm) through a photomask allows for the fixed selection of which features on the wafer will be exposed using a given mask. After exposure to UV light, the wafer was heated, allowing for BOC-deprotection of the exposed features. Subsequent washing, followed the by application of an activated amino acids completes the cycle. With each cycle, a specific amino acid was added to the N-terminus of peptides located at specific locations on the array. These cycles were repeated, varying the mask and amino acids coupled, to achieve the combinatorial peptide library. Thirteen rectangular regions with the dimensions of standard microscope slides, were diced from each wafer. Each completed wafer was diced into 13 rectangular regions with the dimensions of standard microscope slides (25 mm×75 mm). Each of these slides contained 24 arrays in eight rows by three columns. Finally, protecting groups on the side chains of some amino acids were removed using a standard cocktail. The finished slides were stored in a dry nitrogen environment until needed. A number of quality tests are performed ensure arrays are manufactured within process specifications including the use of 3a statistical limits for each step. Wafer batches are sampled intermittently by MALDI-MS to identify that each amino acid was coupled at the correct step, ensuring that the individual steps constituting the combinatorial synthesis are correct. Wafer manufacturing is tracked from beginning to end via an electronic custom Relational Database which is written in Visual Basic and has an access front end with an SQL back end. The front-end user interface allows operators to enter production info into the database with ease. The SQL backend allows us a simple method for database backup and integration with other computer systems for data share as needed. Data typically tracked include chemicals, recipes, time and technician performing tasks. After a wafer is produced the data is reviewed and the records are locked and stored. Finally, each lot is evaluated in a binding assay to confirm performance, as described below.

Assay.

Production quality manufactured microarrays were obtained and rehydrated prior to use by soaking with gentle agitation in distilled water for 1 h, PBS for 30 min and primary incubation buffer (PBST, 1% mannitol) for 1 h. Slides were loaded into an Arraylt microarray cassette (Arraylt, Sunnyvale, Calif.) to adapt the individual microarrays to a microtiter plate footprint. Using a liquid handler, 90 μl of each sample was prepared at a 1:625 dilution in primary incubation buffer (PBST, 1% mannitol) and then transferred to the cassette. This mixture was incubated on the arrays for 1 h at 37° C. with mixing on a TeleShake95 (INHECO, Martinsried, Germany) to drive antibody-peptide binding. Following incubation, the cassette was washed 3× in PBST using a BioTek 405TS (BioTek, Winooski, Vt.). Bound antibody was detected using 4.0 nM goat anti-human IgG (H+L) conjugated to AlexaFluor 555 (Thermo-Invitrogen, Carlsbad, Calif.), or 4.0 nM goat anti-human IgA conjugated to DyLight 550 (Novus Biologicals, Littleton, Colo.) in secondary incubation buffer (0.5% casein in PBST) for 1 h with mixing on a TeleShake95 platform mixer, at 37° C. Following incubation with secondary antibody, the slides were again washed with PBST followed by distilled water, removed from the cassette, sprayed with isopropanol and centrifuged dry. Quantitative signal measurements were obtained by determining a relative fluorescent value for each addressable peptide feature as described below.

Data Acquisition.

Assayed microarrays were imaged using an Innopsys 910AL microarray scanner fitted with a 532 nm laser and 572 nm BP 34 filter (Innopsys, Carbonne, France). The Mapix software application (version 7.2.1) identified regions of the images associated with each peptide feature using an automated gridding algorithm. Median pixel intensities for each peptide feature were saved as a tab-delimitated text file and stored in a database for analysis.

Data Analysis.

Feature intensities were log₁₀ transformed after adding a constant value of 100 to improve homoscedasticity. The intensities on each array were normalized by subtracting the median intensity of the combinatorial library features for that array and adding back the grand median across all samples.

The binding of plasma antibodies to each feature was measured by quantifying fluorescent signal. Peptide features that showed differential signal between groups were determined by t-test of mean peptide intensities with the Welch adjustment for unequal variances. Binding of antibodies in samples from subjects with a first condition were compared to the binding of antibodies in reference samples from subjects having a different second condition, and peptides showing significantly differential signal were identified. A set of peptides that discriminated the first condition from other conditions was identified by comparing mean intensities among patients having the first condition to the mean intensities among subjects with a second, a third, a fourth etc. condition. Peptides that showed significant discrimination i.e. discriminating peptides, were identified based on 5% threshold for false positives after applying the Bonferroni correction for multiplicity (i.e., p<4e-7).

To construct a classifier, features of discriminating peptides were ranked for their ability to differentiate a first condition from a second condition based on the p value associated with a Welch's t-test comparing the first condition to the second, or between the different conditions in a multi-disease model. The number of peptides selected for analysis can vary from less than 10 to more than hundreds or thousands varied and each of the selected peptide features was input to a support vector machine (Cortes C, and Vapnik V. Machine Learning. 1995; 20(3):273-97) with a linear kernel and cost parameter of 0.01 to train a classifier. A five-fold cross validation was repeated 100 times and was used to quantify model performance, estimated as the error under the receiver-operating characteristic curve (AUC) (FIG. 3).

Finally, a fixed SVM classifier was fit in the cohort using the optimal number of features based on performance under cross-validation, selected by their t-test p-values. The SVM classifier was used in assessing reproducibility of the platform.

All analyses were performed using R version 3.2.5. (Team R C. R: A language and environment for statistical computing. R Foundation for Statistical Computing Vienna 2016. Available from: https://www.R-project.org/.)

Peptide Alignment Scoring.

Library peptides were aligned to the human proteome RefSeq release 84, corresponding to human genome build GrCh38 (https://www.ncbi.nlm.nih.gov/refseq/), compiled Mar. 10, 2016, using the longest transcript variant for each unique gene ID. Peptides were aligned to overlapping 20 mer portions of proteome sequences; the overlap was of 10 mer.

The alignment algorithm used a modified BLAST strategy [Altschul SF and Gish W (1996) Methods Enzymol 266: 460-480], requiring a seed of 3 amino acids, a gap penalty of 4 amino acids, and a scoring matrix of BLOSUM62 [Henikoff and, Henikoff J G (1992) Proc Natl Acad Sci USA 89: 10915-10919] modified to reflect the amino acids composition of the array [States D J et al., (1991) Methods 3: 66-70]. These modifications increase the score of similar substitutions, remove penalties for amino acids absent from the array and score all exact matches equally.

To generate an alignment score for a set of discriminating library peptides, peptides that yielded a positive BLAST score were assembled into a matrix, with each row of the matrix corresponding to an aligned peptide and each column corresponding to one of the amino acids in the protein's sequence. Gaps and deletions were permitted within the peptide rows for alignment to the protein. In this way, each position in the matrix received a score associated with the aligned amino acid of the peptide and protein. Each column, corresponding to an amino acid in the protein, was then summed to create an overlap score; this represents coverage of that amino acids position by the classifying peptides. To correct this score for library composition, another overlap score was calculated using an identical method for a list of all array peptides. This allows for the calculation of a peptide overlap difference score, s, at each amino acids position according to the following equation:

s=a−(b/d)*c

In this equation, a is the overlap score from the discriminating peptides, b is the number of discriminating peptides, c is the overlap score for the full library of peptides and d is the number of peptides in the library.

To convert these s scores (which were at the amino acids level) to a full-protein statistic, the sum of scores for every possible tiling 20-mer epitope within a protein is calculated. The final protein score, also known as protein epitope score, S, is the maximum along this rolling overlapping windows of 20 for each protein. A similar set of scores was calculated for 100 iterative-rounds of randomly selecting peptides from the library, equal in number to the number of discriminating peptides. Thep-value for each score, S, is calculated based on the number of times this score is met or exceeded among proteins identified based on alignmnets of the randomly selected peptides, controlling for the number of iterations.

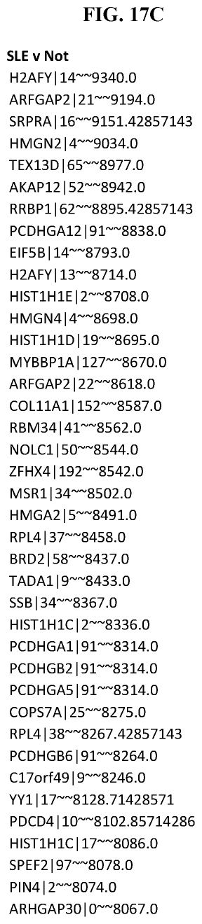

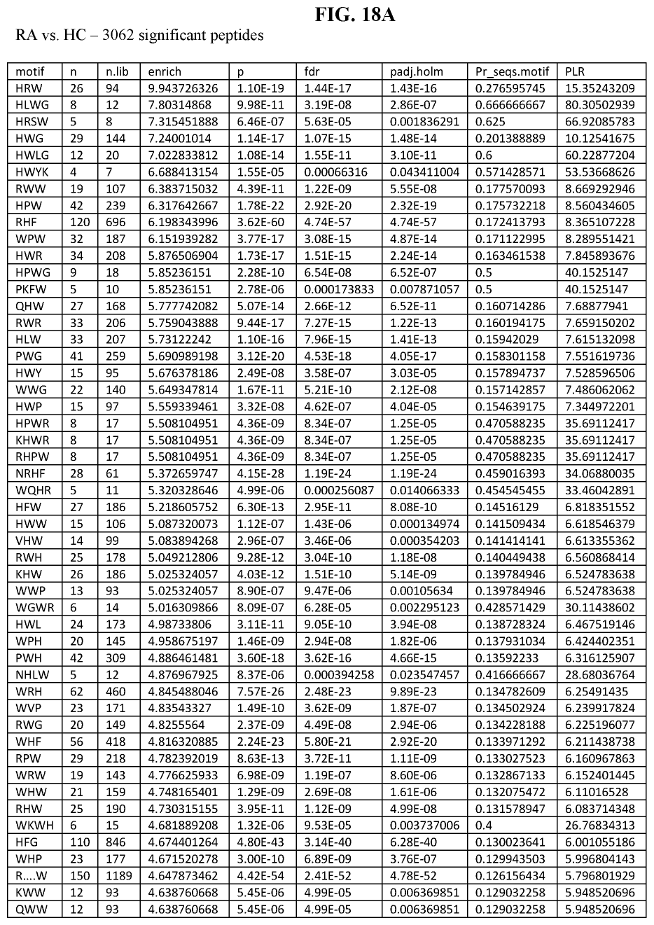

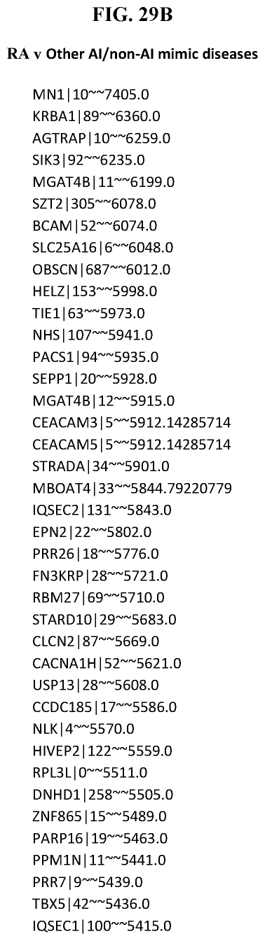

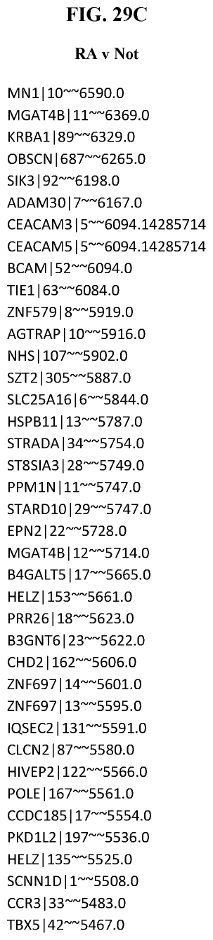

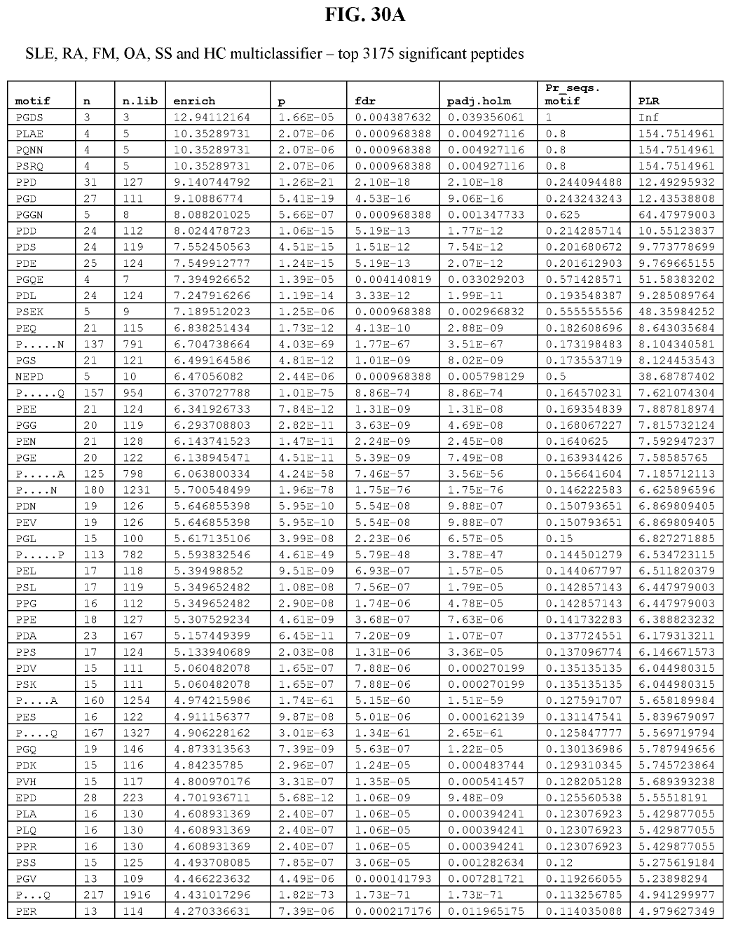

The top 25 candidate biomarkers identified from alignments of discriminating peptides that were determined to distinguish samples from subjects having SLE from samples from healthy subjects (HC), Other AI+non-AI mimic diseases, and Not SLE are shown in FIG. 17A, FIG. 17B, and FIG. 17C, and the top 25 candidate biomarkers identified from alignments of discriminating peptides that were determined to distinguish samples from subjects having RA from samples from healthy subjects (HC), Other AI+non-AI mimic diseases, and Not RA, are shown in FIG. 29A, FIG. 29B, and FIG. 29C, respectively. The candidate biomarkers are listed according to alignment scores.

### Example 2—Differential Diagnosis of SLE

Immunosignatures for differentiating subjects in a group of subjects having SLE alone and SLE in patients with mixed diagnosis from different groups of subjects including healthy controls (HC), “All Disease” (AD), subjects with RA, subjects with OA, subjects with Fibromyalgia (FM), and subjects with Sjogrens. The “All Diseases” comprises non-SLE AI diseases and non-AI mimic diseases.

Immunosignature assays were performed as described in Example 1 and scanned to acquire signal intensity measurements at each feature. Peptide features that showed differential signal between groups were determined by t-test of mean peptide intensities with the Welch adjustment for unequal variances. A binary classifier was developed for each of the contrasts.

Table 1 shows the results for the assay performance for each of the contrasts as AUC values.

TABLE 1
Assay performance for discrimination of SLE

| | | Significant | |
|---|---|---|---|
| Contrast | # Samples | Peptides | cvAUC (95% CI) |
| SLE vs. HC | 134 | 5,121 | 0.90 (0.88-0.92) |
| SLE vs. Other AI + non-AI | 312 | 684 | 0.79 (0.77-0.81) |
| mimic | | | |
| SLE vs. RA | 170 | 201 | 0.80 (0.76-0.85) |
| SLE vs. OA | 99 | 455 | 0.88 (0.86-0.91) |
| SLE vs. Fibromyalgia | 97 | 464 | 0.83 (0.78-0.87) |
| SLE vs. Sjogren's | 95 | 0 | 0.65 (0.60-0.70) |
| SLE vs. Not SLE | 400 | 2042 | 0.81 (0.79-0.83) |

Significant Peptides that discriminated SLE from each of groups were found to be enriched in some amino acids and peptide motifs. FIGS. 4-10 show the motifs (FIGS. 4A-10A) and amino acids (FIGS. 4B-10B) that were enriched in a portion of the discriminating significant peptides in each of the contrasts. The total number of significant i.e. discriminating, peptides identified in the contrasts is indicated in each of the figures. In each of the tables of FIG. 4A, FIG. 4B, FIG. 5A, FIG. 5B, FIG. 6A, FIG. 6B, FIG. 7A, FIG. 7B, FIG. 8A, FIG. 8B, FIG. 9A, FIG. 9B, FIG. 10A, and FIG. 10B:

“n”=the number of times the motif occurs in the top discriminating peptides;

n. lib=the number of times the motif occurs in the array library

“enrich”=the fold enrichment of a motif in the top discriminating peptides relative to the number of times the motif occurs in the array library.

P=the statistical significance of the occurrence of a motif in the top discriminating peptides

Fold enrichment=(no of times a motif (e.g. ABCD) occurs in the list/no of times the motif (ABCD) occurs in the library)/(Total no the motif type (e.g. tetramer) occurs in the list/over total no the motif type (e.g. tetramers) in library). Percent enrichment is “enrichment”×100.

FIG. 4A and FIG. 4B show the peptide motifs (FIG. 4A) and amino acids (FIG. 4B) that are enriched in the peptides that discriminate between the SLE samples from the healthy donor (HC) samples. Comparisons of signal binding data obtained from samples from SLE subjects to binding data from HC group identified peptides that discriminated the SLE samples from the HC group were enriched by greater than 4.2 fold (420%) in one or more motifs listed in FIG. 4A relative to the incidence of the same motifs in the entire peptide library. Additionally, peptides that discriminated SLE samples from HC samples were found to be enriched by greater than 1 (100%) fold in individual amino acids (FIG. 4B).

FIG. 5A and FIG. 5B show the peptide motifs (FIG. 5A) and amino acids (FIG. 5B) that are enriched in the peptides that discriminate between the SLE samples from Other AI+non-AI mimic diseases. Diseases group were enriched by greater than 4.9 fold (490%) in one or more motifs listed in FIG. 5A relative to the incidence of the same motifs in the entire peptide library. Additionally, peptides that discriminated SLE samples from HC samples were found to be enriched by greater than 1.1 (110%) fold in individual amino acids (FIG. 5B).

FIG. 6A and FIG. 6B show the peptide motifs (FIG. 6A) and amino acids (FIG. 6B) that are enriched in the peptides that discriminate between the SLE samples from the “Not SLE” group of samples. Comparisons of signal binding data obtained from samples from SLE subjects to binding data from the “not SLE” group identified peptides that discriminated the SLE samples from the “Not SLE” group were enriched by greater than 5 fold (500% enrichment) in one or more motifs listed in FIG. 6A relative to the incidence of the same motifs in the entire peptide library. Additionally, peptides that discriminated SLE samples from “Not SLE” samples were found to be enriched by greater than 1.00 fold (100% enrichment) in individual amino acids (FIG. 6B).

FIG. 7A and FIG. 7B show the peptide motifs (FIG. 7A) and amino acids (FIG. 7B) that are enriched in the peptides that discriminate between the SLE samples from the RA group of samples. Comparisons of signal binding data obtained from samples from SLE subjects to binding data from HC group identified peptides that discriminated the SLE samples from the RA group were enriched by greater than 3.5 fold (360%) in one or more motifs listed in FIG. 7A relative to the incidence of the same motifs in the entire peptide library. Additionally, peptides that discriminated SLE samples from RA samples were found to be enriched by greater than 1.2 (120%) fold in individual amino acids (FIG. 7B).

FIG. 8A and FIG. 8B show the peptide motifs (FIG. 8A) and amino acids (FIG. 8B) that are enriched in the peptides that discriminate between the SLE samples from the OA group of samples. Comparisons of signal binding data obtained from samples from SLE subjects to binding data from OA group identified peptides that discriminated the SLE samples from the OA group were enriched by greater than 3.8 fold (380%) in one or more motifs listed in FIG. 8A relative to the incidence of the same motifs in the entire peptide library. Additionally, peptides that discriminated SLE samples from OA samples were found to be enriched by greater than 1.2 (120%) fold in individual amino acids (FIG. 8B).

FIG. 9A and FIG. 9B show the peptide motifs (FIG. 9A) and amino acids (FIG. 9B) that are enriched in the peptides that discriminate between the SLE samples from the FM group of samples. Comparisons of signal binding data obtained from samples from SLE subjects to binding data from FM group identified peptides that discriminated the SLE samples from the FM group were enriched by greater than 5 fold (500%) in one or more motifs listed in FIG. 9A relative to the incidence of the same motifs in the entire peptide library. Additionally, peptides that discriminated SLE samples from FM samples were found to be enriched by greater than 1.1 (110%) fold in individual amino acids (FIG. 9B).

FIG. 10A and FIG. 10B shows the peptide motifs (FIG. 10A) and amino acids (FIG. 10B) that are enriched in the peptides that discriminate between the SLE samples from the SS group of samples. Comparisons of signal binding data obtained from samples from SLE subjects to binding data from SS group identified peptides that discriminated the SLE samples from the SS group were enriched by greater than 4.2 fold (420%) in one or more motifs listed in FIG. 10A relative to the incidence of the same motifs in the entire peptide library. Additionally, peptides that discriminated SLE samples from SS samples were found to be enriched by greater than 1.3 (130%) fold in individual amino acids (FIG. 10B).

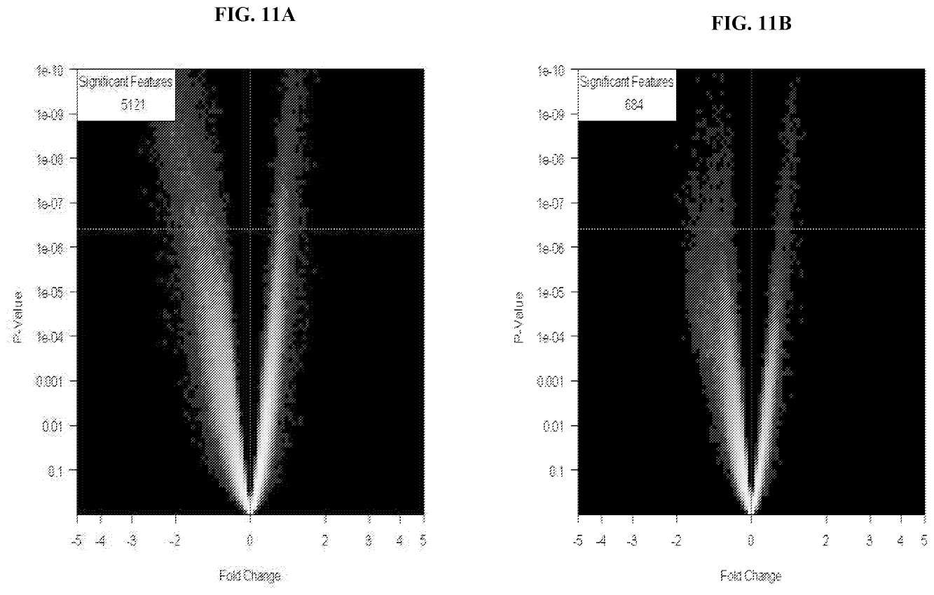

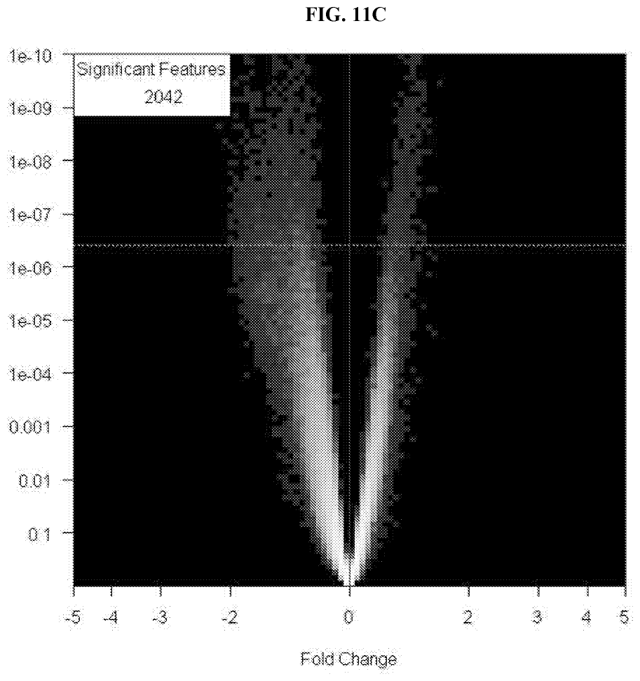

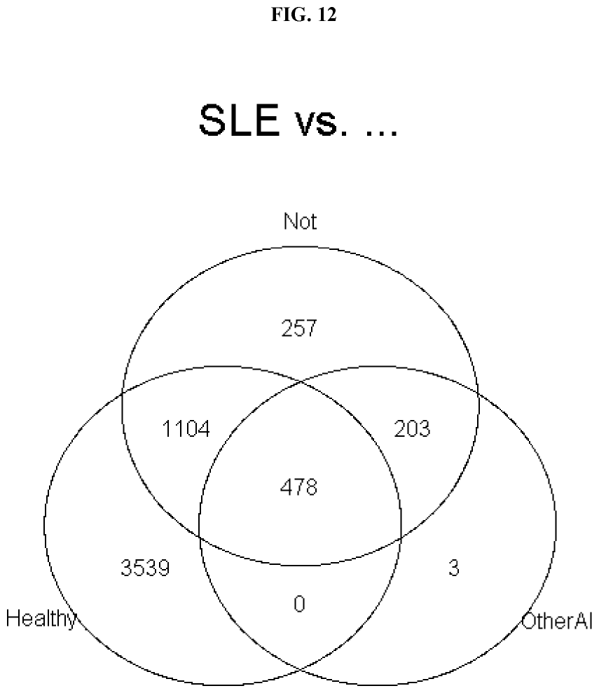

A volcano plot was used to assess the discrimination between samples as the joint distribution of t-test p-values versus log differences in signal intensity means (Fold Change). The density of the peptides at each plotted position is indicated by the heat scale. The peptides above the green dashed line were chosen as discriminating peptides that distinguish between the two groups of each comparison by immunosignature with 95% confidence after applying a Bonferroni adjustment for multiplicity (shown as green line in FIG. 11A, FIG. 11B, and FIG. 11C). The Volcano plots show that the majority of the discriminating peptides displayed lower binding intensities in the All SLE group. FIG. 11A, FIG. 11B, and FIG. 11C respectively show volcano plots of the median-normalized array peptide intensities.

The Welch's t-test identified the significant peptides, which are individual peptides that had significant differences in mean signal between the samples from the SLE group of subjects and the samples from each of the contrast groups. For example, shown in FIG. 11A, FIG. 11B, and FIG. 11C, the Welch's t-test identified 5121 individual peptides that had significant differences in mean signal between the samples from the SLE group of subjects and the samples from the group of healthy donors (FIG. 11A); 684 significant features that displayed differences between SLE group of subjects and the group of subjects having Other AI+non-AI mimic diseases (FIG. 11B); and 2042 significant features that displayed differences between SLE group of subjects and the group of subjects not having SLE i.e. “Not SLE”. Peptides that passed the Bonferroni cut-off in each of eh contrasts are shown in FIG. 12. 478 peptides are common to all contrasts. These 478 peptides comprise two-thirds of the SLE v Other AI+non-AI mimic disease (indicated as “Other AI) contrast, which indicates that these peptides may uniquely identify SLE from similar disorders.

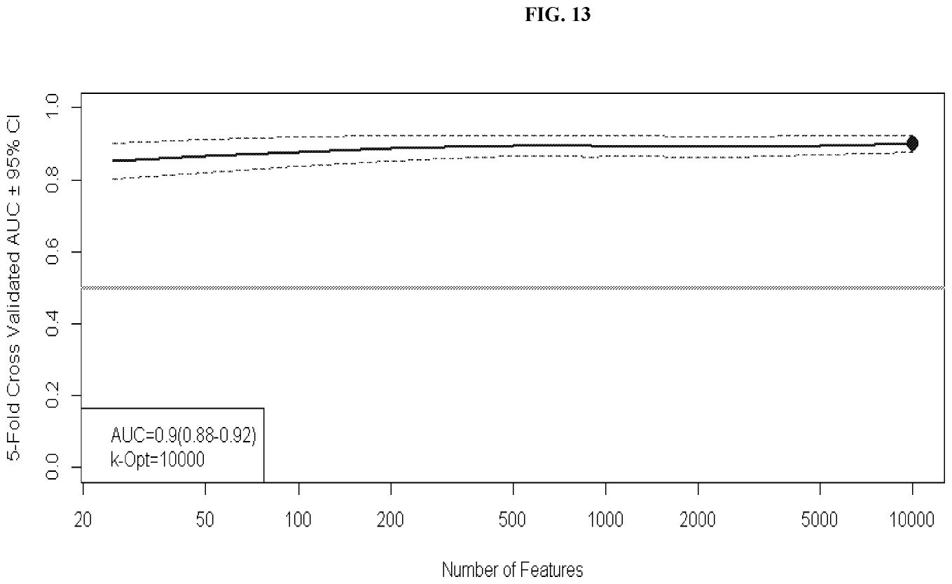

A support vector machine (SVM) classifier was developed for each of the contrasts. Under cross-validation, the best performance (AUC) was determined achieved when the top k peptides, as ranked by Welch t-test were input to the model, where k is allowed to vary between 25 and 10,000 features. FIG. 13 shows the performance of the assay after 100 iterations of five-fold cross validation models, using the top k peptides within each contrast. The optimal k was selected as that k with the highest AUC although AUC itself is very consistent over a wide range of sample sizes. A binary classifier was developed for each of the contrasts. The graph shown in FIG. 13 shows an example that the optimum size of input peptides for each contrast model can be large. For example, the size of input peptides for the contrast of SLE v (HC) was 10000. The graphs also show that the AUCs do not change significantly with increasing number of input peptides.

Support vector machine (SVM) models were used to identify combinations of peptides that can predict the likelihood of SLE versus healthy individuals or other similar diseases. Up to 4000 peptides, as ranked by p-value, were used as SVM inputs. 100 iterations of 5-fold cross-validation minimized the possibility of over-fitting. The histogram in FIG. 14 indicates the area under the receiver operating characteristic curve (AUC) for discriminating between SLE and the listed subgroup: healthy donors (HC), Other AI and non-AI mimic disease (“Other AI”), and the Not SLE group (Other AI+non-AI mimic+HC). The AUC of 0.9 for SLE vs healthy suggests robust discrimination in a diagnostic setting. Discrimination between and SLE and similar diseases can be more difficult, likely because of overlapping etiology and manifestation.

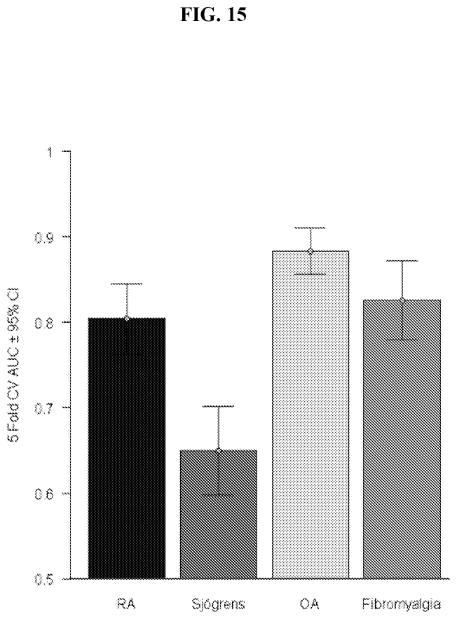

FIG. 15 shows a histogram representing the assay performance in distinguishing SLE from RA, Sjogrens, OA, and FM.

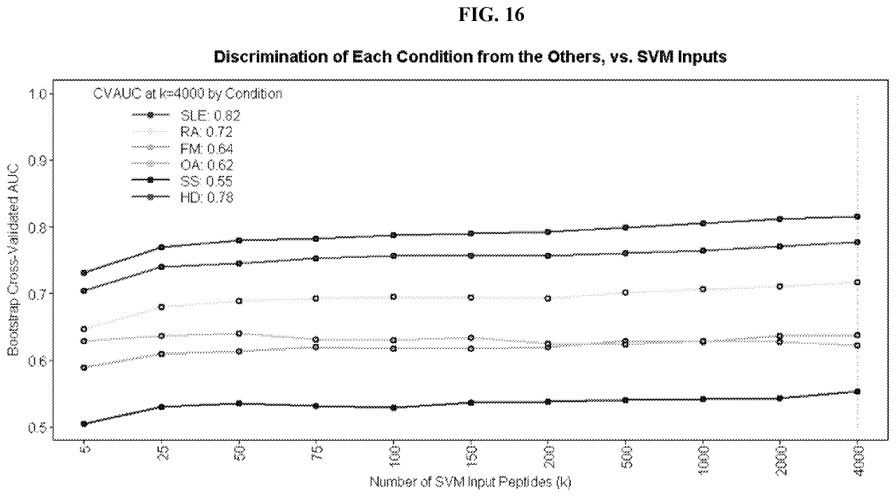

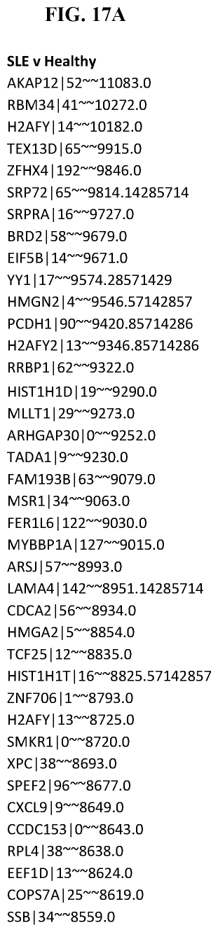

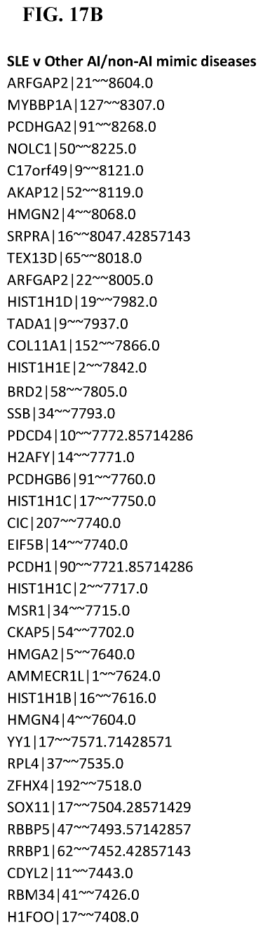

A Multi=class model i.e. simultaneous discrimination of one disease from a group of the remaining related diseases is shown in FIG. 16, yielding AUCs and predictions for these differential diagnoses.

These data show that SLE samples can be discriminated from healthy samples with an AUC of 0.9. These data also show that SLE was easily distinguished from non-autoimmune disease (OA and Fibromyalgia) and from Sjögren's. Additionally, the data also show that SLE can be distinguished from samples of patients having Other AI+non-AI mimic diseases.

Thus, the immunosignature (IS) technology can be used to classify subjects with SLE from healthy controls or subjects with diseases that have common symptoms or underlying immunological dysregulation.

### Example 3—Proteome Mapping the SLE-Classifying Peptides Identifies Candidate Biomarkers of SLE

Significant discriminating peptides identified by the contrasts described in Example 2 were used to identify candidate biomarkers.

Significant peptides associated with SLE were mapped to putative antigens that included a known immunogenic epitope of SSB.

The library peptides that significantly distinguished SLE from healthy subjects, Other AI+non-AI mimic diseases, and “Not SLE” subjects were aligned to the human proteome RefSeq release 84, corresponding to human genome build GrCh38 (https://www.ncbi.nlm.nih.gov/refseq/), compiled Mar. 10, 2016, using the longest transcript variant for each unique gene ID, with a modified BLAST algorithm and scoring system that used a sliding window of overlapping 20-mers (Example 1). Peptides were aligned to 20mer segments of the proteins overlapping by 10 mer. The resulting ranked list of the top 25 candidate biomarkers protein-target regions provided in FIG. 17A, FIG. 17B, and FIG. 17C. The gene name|epitope start ˜˜alignment scores are provided. These classifying peptides display a high frequency of alignment scores that greatly exceed the maximum scores obtained by performing the same analysis with ten equally-sized sets of peptides that were randomly selected from the library.

Among the top-scoring candidates mapped by the SLE classifying peptides was the surface membrane translocated La/SSB antigen. Notably, the known and clinically used SLE autoantigen SSB is highly ranked on each list. Specifically, one of three immunodominant epitopes, contained in the amino acids at positions 340-360, is identified. The SSB autoantigen maps to amino acids 340-360 of the immunodominant epitope of the intracellular human La protein, which is redistributed from the nucleus to the cell surface, following loss of the nuclear localization signal, during apoptosis [Neufing et al. (2005), Exposure and binding of selected immunodominant La/SSB epitopes on human apoptotic cells. Arthritis & Rheumatism, 52: 3934-3942. doi:10.1002/art.21486] (FIG. 17A, FIG. 17B, and FIG. 17C).

Other top scoring candidate biomarkers mapped by the SLE discriminating peptides included histone proteins. Histones are important target antigens of nuclear antibodies, and anti-nuclear antibodies (ANA), and anti-histone antibody tests are typically performed in detecting autoantibodies that are relevant to the diagnosis of SLE [Manson and Rahman (2006), Systemic Lupus Erythematosus. Orphanet Journal of Rare Diseases 1:6. doi 10.1186/1750-1172-1-6] (FIG. 17A, FIG. 17B, and FIG. 17C).

Another top scoring candidate biomarker mapped by the SLE discriminating peptides was identified as the HMGN https://www.ncbi.nlm.nih.gov/pubmed/8318042?dopt=Abstract.

Together the 25 candidate proteome targets in each contrast accounted for the aligned discriminating peptides. Leading candidate biomarkers can also be identified by up to all of the total number of discriminating peptides.

These data show that array peptides that mimic SLE autoantigen epitopes were bound differentially by peripheral blood antibodies in SLE subjects. These discriminating peptides were mapped to several known markers of SLE. Other listed candidate targets could be novel markers of SLE.

### Example 4—Differential Diagnosis of RA

Immunosignatures (IS) were obtained for differentiating subjects in a group of RA subjects having RA from groups of subjects including healthy controls (HC), subjects having other rheumatic diseases (ORD), SLE, OA, Fibromyalgia (FM), Sjogrens (SS), a group of subjects with Other AI/non-AI mimic diseases, and Not RA subjects. The Other rheumatic Disease group (ORD) (239) consisted of: RA, SS, OA, psoriatic arthritis, gout, seronegative spondyloarthropathy, and pseudogout. Subjects with rheumatological diseases were diagnosed based on ACR criteria.

The assays were performed as described in Example 1 and scanned to acquire signal intensity measurements at each feature. Peptide features that showed differential signal between groups were determined by t-test of mean peptide intensities with the Welch adjustment for unequal variances, as described previously.

Table 2 shows the results for the assay performance for each of the contrasts as AUC values.

TABLE 2
Assay performance for discrimination of RA

| | # | Significant | cvAUC |
|---|---|---|---|
| Contrast | Samples | Peptides | (95% CI) |
| RA vs. HC | 154 | 3,062 | 0.80 (0.78-0.83) |
| RA vs. other rheumatic diseases\hat{ } | 239 | 328 | 0.70 (0.66-0.74) |
| RA vs. SLE | 170 | 201 | 0.80 (0.76-0.85) |
| RA vs. OA | 119 | 130 | 0.73 (0.67-0.78) |
| RA vs. Fibromyalgia | 117 | 753 | 0.78 (0.73-0.83) |
| RA vs. SS | 115 | 20 | 0.66 (0.60-0.73) |
| RA vs. Other AI + nonAI mimic | 341 | 742 | 0.70 (0.66-0.73) |
| RA vs. Not RA | 400 | 1564 | 0.70 (0.67-0.72) |

\hat{ }Other rheumatic diseases = SLE, SS, OA, psoriatic arthritis, gout, pseudogout, serospan

Significant Peptides that discriminated RA from each of groups were found to be enriched in some amino acids and peptide motifs. FIGS. 18-24 show the motifs (FIGS. 18A-24A) and amino acids (FIGS. 18B-24B) that were enriched in a portion of the discriminating significant peptides in each of the contrasts. The total number of significant peptides is indicated in each of the figures.

FIG. 18A and FIG. 18B show the peptide motifs (FIG. 18A) and amino acids (FIG. 18B) that are enriched in the peptides that discriminate between the RA samples from the healthy donor (HC) samples. Comparisons of signal binding data obtained from samples from SLE subjects to binding data from HC group identified peptides that discriminated the SLE samples from the HC group were enriched by greater than 4.6 fold (460%) in one or more motifs listed in FIG. 18A relative to the incidence of the same motifs in the entire peptide library. Additionally, peptides that discriminated SLE samples from HC samples were found to be enriched by greater than 1 (100%) fold in individual amino acids (FIG. 18B).

FIG. 19A and FIG. 19B show the peptide motifs (FIG. 19A) and amino acids (FIG. 19B) that are enriched in the peptides that discriminate between the RA samples from the “other Rheumatic Diseases” (ORD) group of samples. Comparisons of signal binding data obtained from samples from RA subjects to binding data from ORD group identified peptides that discriminated the RA samples from the ORD group were enriched by greater than 4.8 fold (480%) in one or more motifs listed in FIG. 19A relative to the incidence of the same motifs in the entire peptide library. Additionally, peptides that discriminated RA samples from ORD samples were found to be enriched by greater than 1.1 (110%) fold in individual amino acids (FIG. 19B).

FIG. 20A and FIG. 20B shows the peptide motifs (FIG. 20A) and amino acids (FIG. 20B) that are enriched in the peptides that discriminate between the RA samples from the “Not RA” group of samples. Comparisons of signal binding data obtained from samples from RA subjects to binding data from “Not RA” group identified peptides that discriminated the RA samples from the “Not RA” group were enriched by greater than 4.9 fold (492%) in one or more motifs listed in FIG. 20A relative to the incidence of the same motifs in the entire peptide library. Additionally, peptides that discriminated RA samples from “not RA” samples were found to be enriched by greater than 1.1 (110%) fold in individual amino acids (FIG. 20B).

FIG. 21A and FIG. 21B show the peptide motifs (FIG. 21A) and amino acids (FIG. 21B) that are enriched in the peptides that discriminate between the RA samples from the “Other AI+non-AI mimic diseases” group of samples. Comparisons of signal binding data obtained from samples from RA subjects to binding data from the Other AI group identified peptides that discriminated the RA samples from the Other AI+non-AI mimic diseases group were enriched by greater than 4.8 fold (480%) in one or more motifs listed in FIG. 21A relative to the incidence of the same motifs in the entire peptide library. Additionally, peptides that discriminated RA samples from the Other AI+non-AI mimic diseases samples were found to be enriched by greater than 1 (100%) fold in individual amino acids (FIG. 21B).

FIG. 22A and FIG. 22B shows the peptide motifs (FIG. 22A) and amino acids (FIG. 22B) that are enriched in the peptides that discriminate between the RA samples from the OA group of samples. Comparisons of signal binding data obtained from samples from RA subjects to binding data from OA group identified peptides that discriminated the RA samples from the OA group were enriched by greater than 3.3 fold (330%) in one or more motifs listed in FIG. 22A relative to the incidence of the same motifs in the entire peptide library. Additionally, peptides that discriminated RA samples from OA samples were found to be enriched by greater than 1.6 (156%) fold in individual amino acids (FIG. 22B).

FIG. 23A and FIG. 23B shows the peptide motifs (FIG. 23A) and amino acids (FIG. 23B) that are enriched in the peptides that discriminate between the RA samples from the FM group of samples. Comparisons of signal binding data obtained from samples from RA subjects to binding data from FM group identified peptides that discriminated the RA samples from the FM group were enriched by greater than 3.9 fold (390%) in one or more motifs listed in FIG. 23A relative to the incidence of the same motifs in the entire peptide library. Additionally, peptides that discriminated RA samples from FM samples were found to be enriched by greater than 1.1 (110%) fold in individual amino acids (FIG. 23B).

FIG. 24A and FIG. 24B shows the peptide motifs (FIG. 24A) and amino acids (FIG. 24B) that are enriched in the peptides that discriminate between the RA samples from the SS group of samples. Comparisons of signal binding data obtained from samples from RA subjects to binding data from SS group identified peptides that discriminated the RA samples from the SS group were enriched by greater than 4.2 fold (420%) in one or more motifs listed in FIG. 24A relative to the incidence of the same motifs in the entire peptide library. Additionally, peptides that discriminated RA samples from SS samples were found to be enriched by greater than 1.3 (130%) fold in individual amino acids (FIG. 24B).

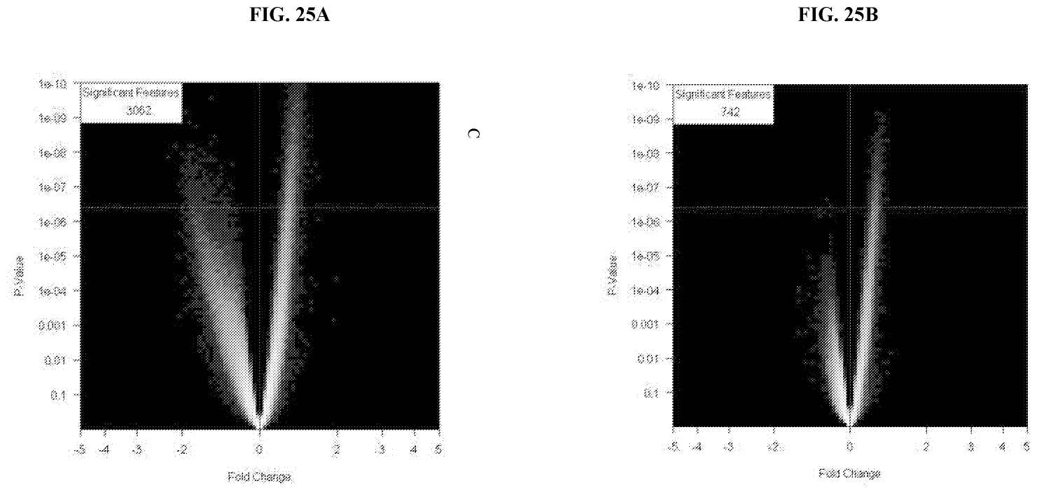

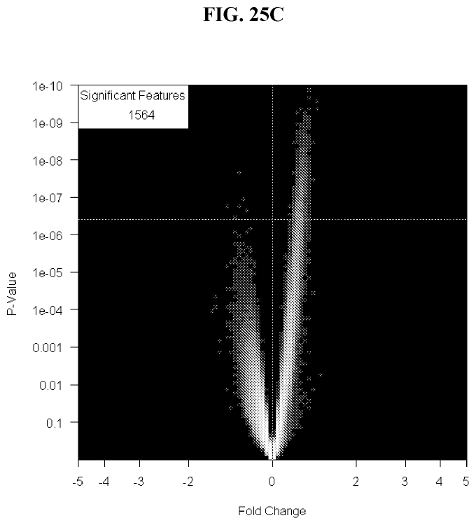

As described for the SLE contrasts, volcano plots were used to assess the discrimination between samples as the joint distribution of t-testp-values versus log differences in signal intensity means (Fold Change). The density of the peptides at each plotted position is indicated by the heat scale. The peptides above the green dashed line were chosen as discriminating peptides that distinguish between the two groups of each comparison by immunosignature with 95% confidence after applying a Bonferroni adjustment for multiplicity (shown as green line in FIG. 25A, FIG. 25B, and FIG. 25C). FIG. 25A, FIG. 25B, and FIG. 25C respectively show volcano plots of the median-normalized array peptide intensities.

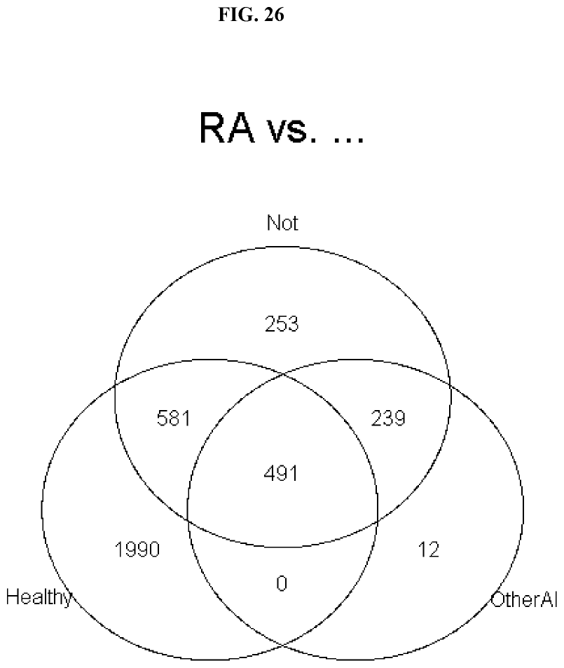

The Welch's t-test identified the significant peptides, which are individual peptides that had significant differences in mean signal between the samples from the RA group of subjects and the samples from each of the contrast groups. For example, shown in FIG. 25A, FIG. 25B, and FIG. 25C, the Welch's t-test identified 3062 individual peptides that had significant differences in mean signal between the samples from the RA group of subjects and the samples from the group of healthy donors (FIG. 25A); 742 significant features that displayed differences between RA group of subjects and the group of subjects having “All Diseases” i.e. Other AI+non-AI mimic diseases (FIG. 25B); and 1564 significant features that displayed differences between RA group of subjects and the group of subjects not having RA i.e. “Not RA”. Peptides that passed the Bonferroni cut-off in each of the contrasts are shown in FIG. 26. 491 peptides are common to all contrasts. These 491 peptides comprise two-thirds of the RA v Other AI+non-AI mimic diseases indicated as “Other AI” contrast, which indicates that these peptides may uniquely identify RA from similar disorders.

Significant peptides were identified by Welch's t-test and support vector machine (SVM) classifier was developed for each of the contrasts, as described in Example 2. Support vector machine (SVM) models were used to identify combinations of peptides that can predict the likelihood of RA versus healthy individuals or other similar diseases. Up to 4000 peptides, as ranked by p-value, were used as SVM inputs. 100 iterations of 5-fold cross-validation minimized the possibility of over-fitting.

The histogram in FIG. 27 indicates the area under the receiver operating characteristic curve (AUC) for discrimination between RA and the listed subgroup: healthy donors (HC), Other AI and non-AI mimic disease (“Other AI”), and the Not SLE group (Other AI+non-AI mimic+HC). The AUC of 0.9 for SLE vs healthy suggests robust discrimination in a diagnostic setting. The AUC of 0.8 for RA vs healthy indicates discrimination in a diagnostic setting.

Comparisons of signal intensities of array-bound antibodies from samples of subjects with RA showed that RA could be distinguished from other AI and non-AI mimic diseases (Table 2).

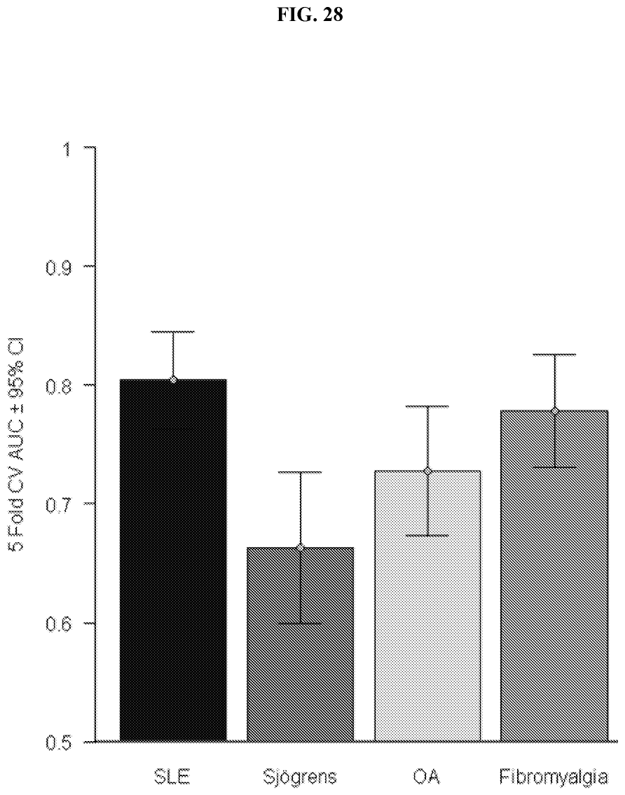

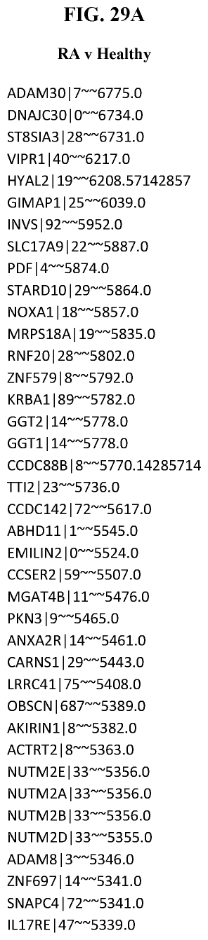

A histogram depicting the assay performance in distinguishing RA samples from SLE, Sjogrens, OA and Fibromyalgia is provided in FIG. 28.

Using IS technology, RA is best discriminated from distinct conditions, including patients with lupus and healthy controls. Nevertheless, RA can also be differentiated from closely-related conditions such as SS with modest cvAUCs. The results indicate that IS technology could provide a single test using a small serum sample capable of multi-classification across a range of symptomatically related diseases, or in patients with conditions referred to rheumatologic evaluation.

### Example 5—Proteome Mapping the RA-Classifying Peptides Identifies Candidate Biomarkers of SLE

The top 1000 library peptides, as ranked by p-value) that significantly distinguished RA from healthy subjects, Other AI+non-AI mimic diseases, and “Not RA” subjects, as described in Example 4, were aligned to the human proteome RefSeq release 84, corresponding to human genome build GrCh38 (https://www.ncbi.nlm.nih.gov/refseq/), compiled Mar. 10, 2016, using the longest transcript variant for each unique gene ID, with a modified BLAST algorithm and a BLOSUM62-based scoring system that used a sliding window of overlapping 20-mers (Example 1). Peptides were aligned to 20mer segments of the proteins overlapping by 10 mer. The resulting ranked list of the top 25 candidate protein-target regions provided in FIG. 29A, FIG. 29B, and FIG. 29C. The gene name|epitope start ˜˜alignment scores are provided.

These classifying peptides display a high frequency of alignment scores that greatly exceed the maximum scores obtained by performing the same analysis with ten equally-sized sets of peptides that were randomly selected from the library.

Among the top-scoring candidates mapped by the RA classifying peptides was the MN1 autoantiboides associated with BrCA cancers [Wang, et al. “Plasma autoantibodies associated with basal-like breast cancers”, Cancer Epidemiol Biomarkers Prey. 2015 September; 24(9): 1332-1340.

Together the 25 candidate proteome targets in each contrast accounted for the aligned discriminating peptides. Leading candidate biomarkers can also be identified by up to all of the total number of discriminating peptides.

These data show that array peptides that array peptides, which mimic RA autoantigen epitopes, were bound differentially by peripheral blood antibodies in RA subjects. These discriminating peptides were mapped to several markers that could be novel markers of RA.

### Example 6—Simultaneous Classification of Different Health Conditions

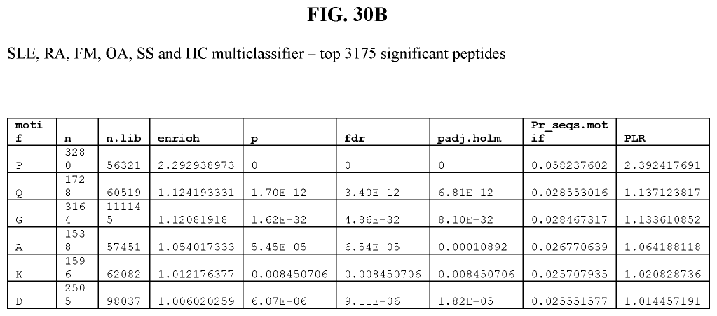

Peptides simultaneously discriminating SLE, RA, FM, OA, SS and HC from each other in the multiclassifier analysis were enriched by greater than 100% in one or more motifs listed in FIG. 30A relative to the incidence of the same motifs in the entire peptide library. Additionally, the peptides that discriminated SLE, RA, FM, OA, SS and HC samples from each other in the multiclassifier analysis were enriched by greater than 100% in one or more amino acids listed in FIG. 30B.

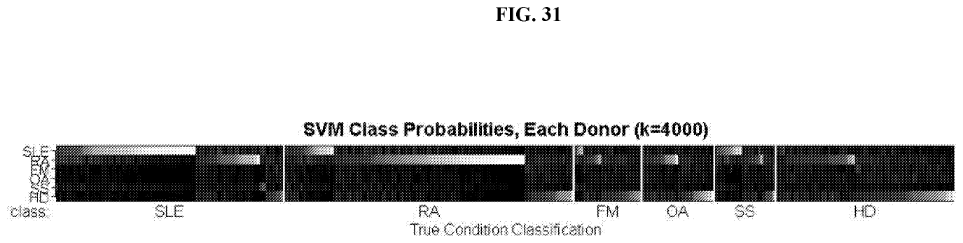

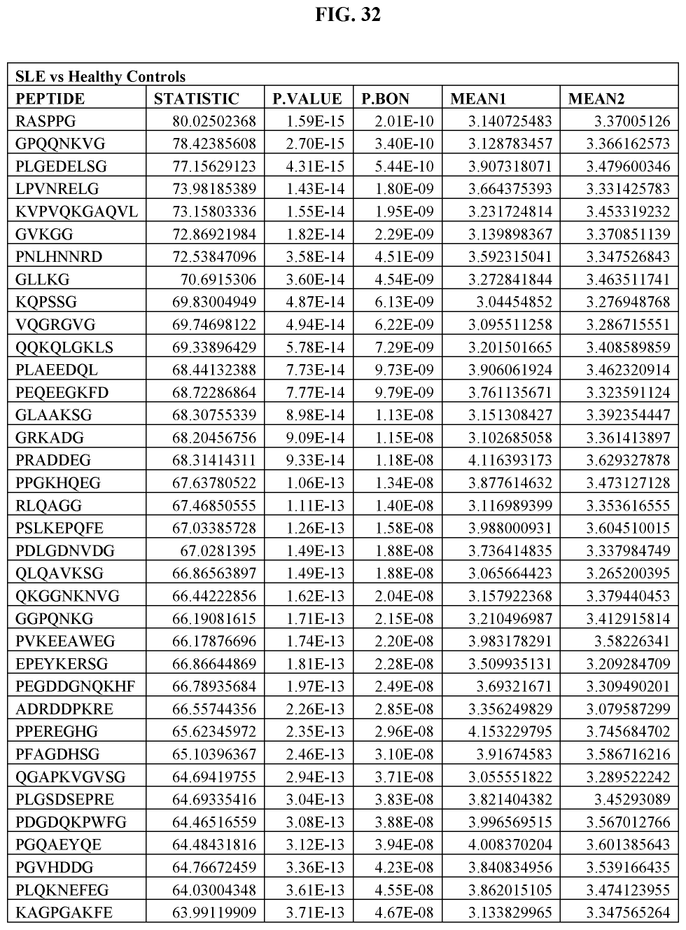

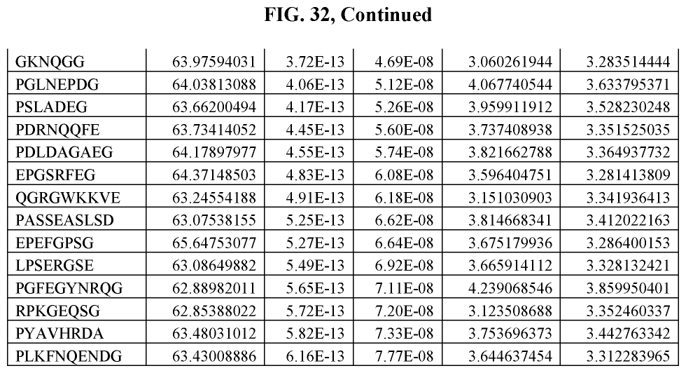

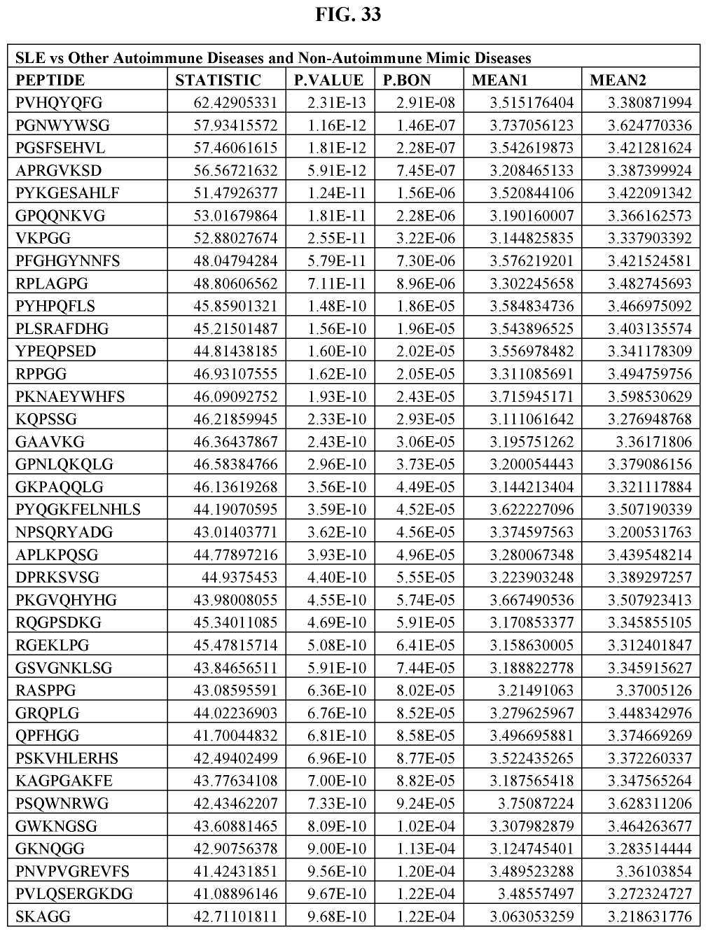

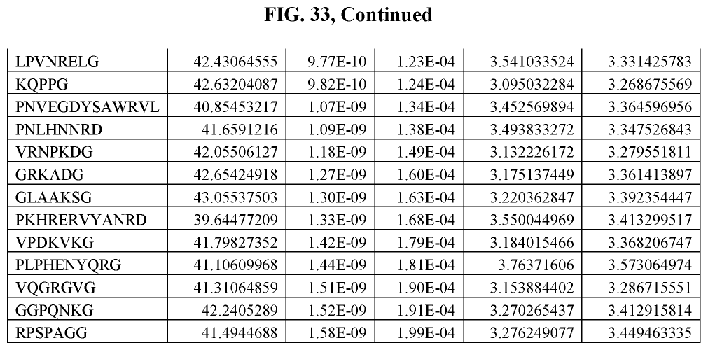

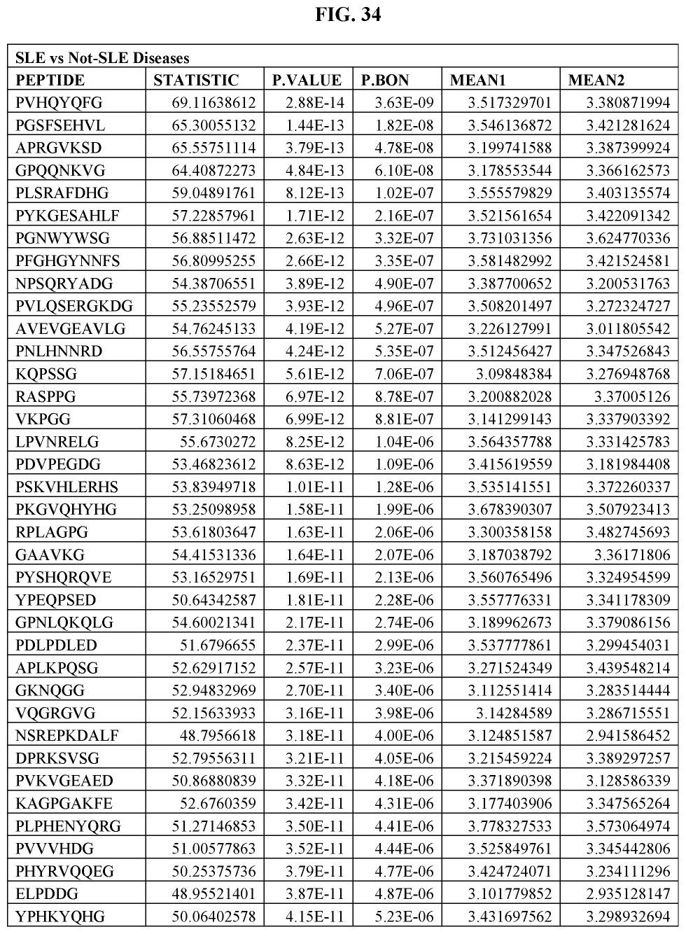

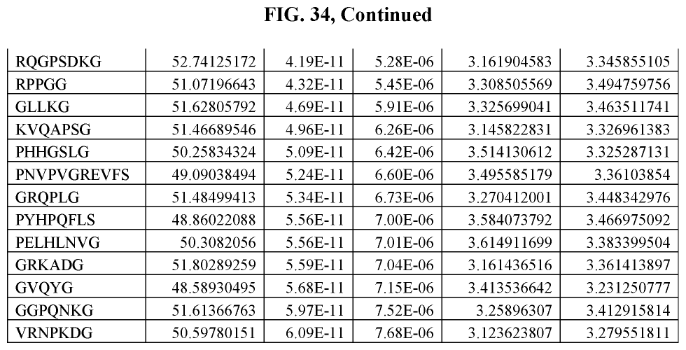

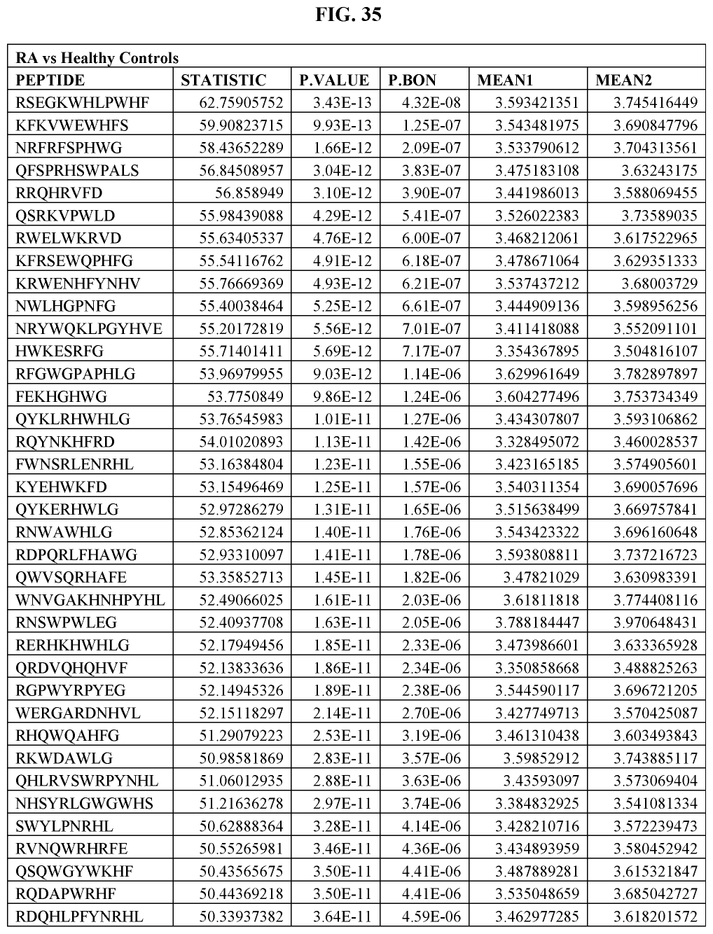

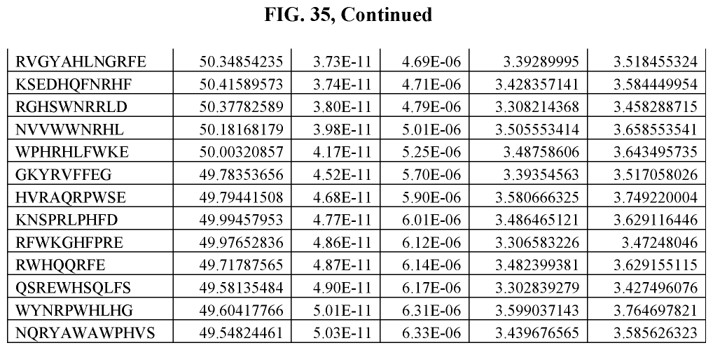

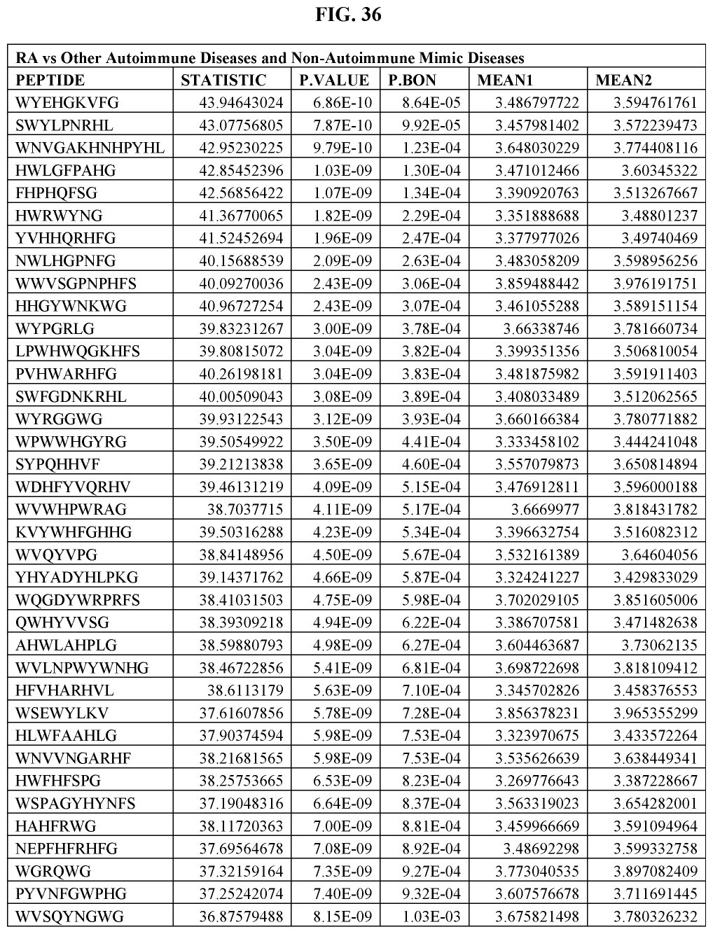

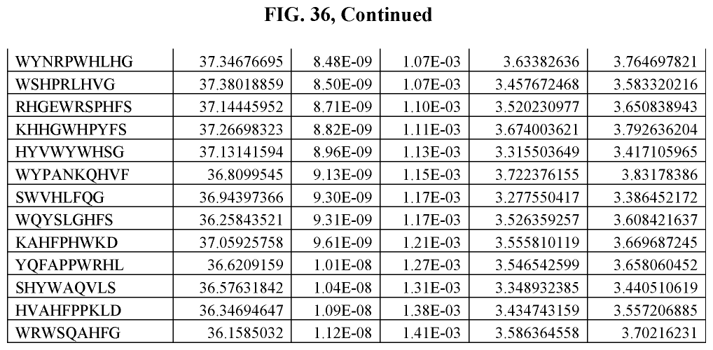

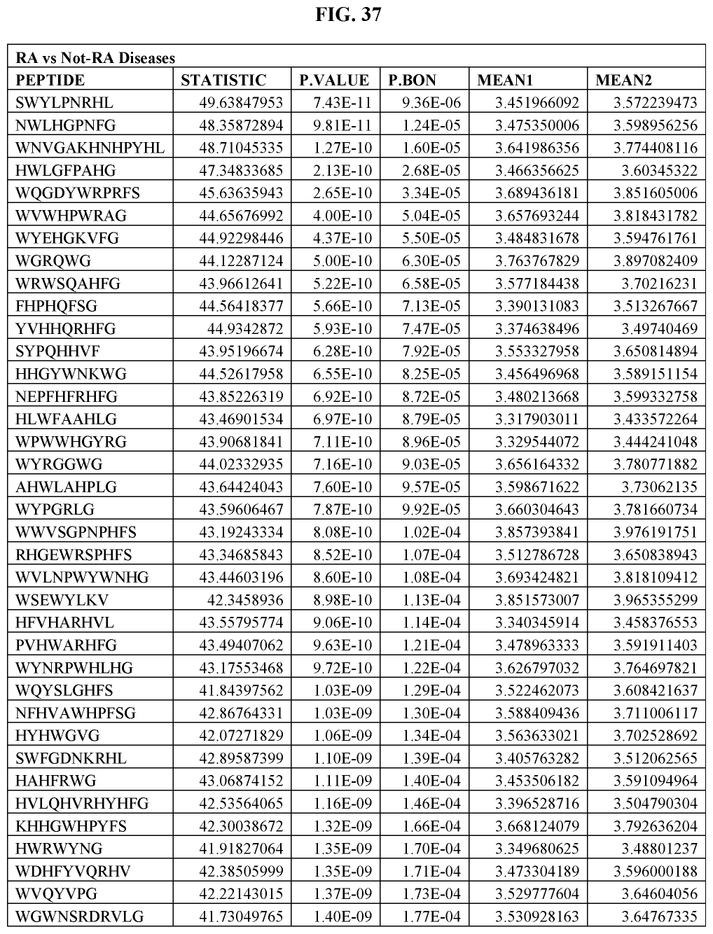

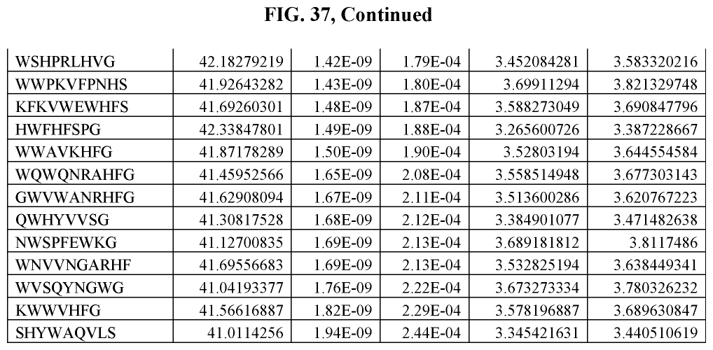

The heat map shown in FIG. 31 visualizes the mean predicted probability of class membership of out of the bag cross validation model predictions for each of the test cohort samples, encompassing all six conditions. Each sample has a predicted class membership for each outcome ranging from 0 (black) to 100% (white).

These data show that the immunosignature assay can simultaneously distinguish one health condition from two or more other conditions.

### Example 7—Automated Assay and Imaging System

Peptide microarray assay and imaging process is an automated system. The system is has two subsystems that automate key functions: an Automated Assay System and an Automated Imaging System. The process has four steps. The Automated Assay System is responsible for the first three steps: sample reformatting, sample dilution, and assay running. The Automated Imaging System is responsible for automating the imaging process. The sample reformatting and sample dilution are intended to be run as separate preparatory protocols to set up samples in advance of an assay.

Sample reformatting is done 1-96 samples at a time. The system generates mapping output files (in *.xls or *.csv format, for example) of the barcoded samples and their reformatted locations in barcoded plate(s) containing sample plate location and barcode information. This information is used in downstream protocols for sample tracking and chain of custody. It also generates human friendly report files such as formatted Excel documents. An exemplary sample reformatting protocol is provided in Table 3 below:

TABLE 3
Sample Reformatting Process

| Step | Process - Sample Reformatting |
|---|---|
| 1. | Load system with samples in tubes (1-96 samples) |
| 2. | Load system with Sample Destination Plate |
| 3. | Load tips |
| 4. | Register barcodes |
| 5. | Pick up 8 tips |
| 6. | Aspirate 30 uL of sample from 8 tubes |
| 7. | Dispense 30 uL of sample into 8 positions in the Sample |
| | Destination Plate |
| 8. | Eject tips |
| 9. | Repeat steps 5-8 eleven more times to transfer all 96 samples |

In brief, sample reformatting includes loading the samples in their original container, such as a tube, and loading the destination plate. Tips are provided to the system to transfer the samples from the tubes to the plate. Barcodes are used to track the samples. In groups of 8, samples are transferred from tubes to wells of the destination plate until all of the samples are transferred to the plate.

An exemplary sample dilution protocol is provided in Table 4 below:

TABLE 4
Sample Dilution Process

| Step | Process - Sample Dilution |
|---|---|
| 1. | Load system with sample plate from sample reformatting step |
| 2. | Load system with two empty dilution plates |
| 3. | Load system with tips |
| 4. | Pickup dilution buffer tips. Transfer 360 uL dilution buffer to |
| | plate 1, and 360 uL buffer to plate 2. Eject tips |
| 5. | Pickup new tips. Transfer 15 uL of sample into plate 1. Mix |
| | rapidly for 15 cycles with 250 uL with dual height mixing at |
| | maximum speed. Eject tips to waste |
| 6. | Pickup new tips. Aspirate 15 uL of intermediate dilution and |
| | dispense to plate 2. Mix rapidly for 15 cycles with 250 ul with |
| | dual height mixing at maximum speed |
| 7. | Eject tips |

In brief, the samples from the previous step, in a plate, are loaded into the system along with dilution plates and tips for transferring liquid from the original plate to the diluted plate. Samples are serially diluted in two steps: a portion of the original plate is put into a volume of buffer, mixed, and then a portion of the first dilution plate is placed into a volume of buffer in the second dilution plate.

In some cases, samples are further diluted. An exemplary sample dilution protocol is provided in Table 5 below:

TABLE 5
Sample Dilution Process

| Step | Process - Sample Dilution |
|---|---|
| 1. | Load system with Intermediate dilution plate from previous |
| | Standard Sample Dilution |
| 2. | Load system with an empty final dilution plate |
| 3. | Load system with tips |
| 4. | Pickup dilution buffer tips. Transfer 360 uL dilution buffer to |
| | emplty plate. Eject tips |
| 5. | Pickup new tips. Transfer 15 uL of sample into plate 1. Mix |
| | rapidly for 15 cycles with 250 ul with dual height mixing at |
| | maximum speed. Eject tips to waste |

Some samples are serially diluted to optimize concentration of the sample in the assay. An exemplary serial dilution process is provided in Table 6 below:

TABLE 6
Serial Dilution Process

| Step | Process - Sample Dilution |
|---|---|
| 1. | Load system with sample plate from sample reformatting step |
| 2. | Load system with appropriate number of empty dilution plates |
| | for serial dilution steps |
| 3. | Load system with appropriate number of tips for serial dilution |
| | steps |
| 4. | Pickup dilution buffer tips. Transfer appropriate volume of |
| | dilution buffer to dilution plates. Eject tips |
| 5. | Pickup new tips. Transfer appropriate volume of sample into first |
| | plate of serial dilution. Mix rapidly for 15 cycles with 50% |
| | volume with dual height mixing at maximum speed. |
| | Eject tips to waste |
| 6. | Repeat for all steps in the serial dilution |

An assay is run on the diluted samples. An exemplary assay protocol is provided in Table 7 below:

TABLE 7
Assay Process

| Step | Process - Assay |
|---|---|
| 1. | Load system with assay cassettes (1-6) |
| 2. | Pickup new tips. Transfer 95 uL of diluted serum from a sample |
| | dilution protocol and reverse pipette 90 ul into cassette. |
| | Eject tips to waste |
| 3. | Move plate to sealer and apply seal. |
| 4. | Move plate to Teleshake and incubate for 60 minutes |
| 5. | After incubation is complete, move plate to Xpeel and remove |
| | seal. Move plate to plate washer |
| 6. | Wash plate for 3 minutes. Move plate to deck |
| 7. | Pickup tips. Aspirate 45 uluL of secondary buffer and reverse |
| | pipette 40 uL into assay cassette. Eject tips to waste |
| 8. | Move plate to sealer and apply seal. |
| 9. | Move plate to Teleshake and incubate for 60 minutes |
| 10. | Move plate to Xpeel and remove seal |
| 11. | Move plate to washer and wash for 4 minutes |
| 12. | Move plate to output stack |

Briefly, the system is loaded with assay cassettes and plates loaded with diluted sample. The diluted samle is applied to the cassettes. The cassettes are sealed and shaken/incubated for an hour. The casettes are unsealed and washed. A secondary antibody is added to the cassette, sealed, and shaken/incubated for an hour. The cassette is washed and then is ready for imaging.

Once the assay is run, imaging captures the results of the assay. An exemplary imaging protocol is provided in Table 8 below:

| Step | Process - Automated Imaging |
|---|---|
| 1 | Load system with Imaging cassettes |
| 2 | Specify run parameters |
| 3 | Load cassette |
| 4 | Initiate Imaging run |
| 5 | Unload Cassette |
| 6 | Repeat Steps 3-5 until all cassettes are complete |

What is claimed is:

1. A method of making a differential diagnosis of an autoimmune disease, said method comprising:
(a) contacting a sample from a subject to an array of peptides comprising at least 10,000 different peptides;
(b) detecting the binding of antibodies present in said sample to at least 25 peptides on said array to obtain a combination of binding signals; and
(c) comparing said combination of binding signals to one or more groups of combination of reference binding signals, wherein at least one of said group of combination of reference binding signals are obtained from a plurality of reference subjects known to have a disease different from the autoimmune disease of the subject to enable the differential diagnosis of said subject for the autoimmune disease, wherein the method performance is characterized by an area under the receiver operator characteristic (ROC) curve (AUC) between the autoimmune disease and each of the group of combinations of reference binding signals being greater than 0.6.

2. The method of claim 1, further comprising:
(i) identifying a combination of differentiating reference binding signals, wherein said differentiating reference binding signals distinguish samples from reference subjects known to have said autoimmune disease from samples from reference subjects known to have said disease different from the autoimmune disease; and
(ii) applying the combination of differentiating reference binding signals to step 1(c) to enable differential diagnosis of the autoimmune disease.

3. The method of claim 2, wherein each of said combination of differentiating reference binding signals is obtained by detecting the binding of antibodies present in a sample from each of said plurality of reference subjects to said at least 25 peptides on an array of peptides comprising at least 10,000 different peptides in step (a) of claim 1.

4. The method of claim 2, wherein the difference between said combination of binding signals and said combination of said reference binding signals to said at least 25 peptides determines said differential diagnosis.

5. The method of claim 1, further comprising:
(d) comparing said combination of binding signals to a reference binding signal obtained from a plurality of reference subjects known to have the autoimmune disease.

6. The method of claim 1, wherein said different disease is an autoimmune disease.

7. The method of claim 1, wherein said different disease is a non-autoimmune mimic disease.

8. The method of claim 6, wherein said autoimmune disease is systemic lupus erythematosus (SLE), and wherein said different autoimmune disease is rheumatoid arthritis (RA).

9. The method of claim 8, wherein said discriminating peptides are enriched by greater than 100% in one or more sequence motifs listed in FIG. 7A.

10. The method of claim 8, wherein said discriminating peptides are enriched by greater than 100% in one or more amino acids listed in FIG. 7B.

11. The method of claim 6, wherein said autoimmune disease is systemic lupus erythematosus, and wherein said different non-autoimmune-disease is osteoarthritis (OA).

12. The method of claim 11, wherein said discriminating peptides are enriched by greater than 100% in one or more sequence motifs listed in FIG. 8A.

13. The method of claim 11, wherein said discriminating peptides are enriched by greater than 100% in one or more amino acids listed in FIG. 8B.

14. The method of claim 6, wherein said autoimmune disease is systemic lupus erythematosus, and wherein said different non-autoimmune disease is fibromyalgia (FM).

15. The method of claim 14, wherein said discriminating peptides are enriched by greater than 100% in one or more sequence motifs listed in FIG. 9A.

16. The method of claim 14, wherein said discriminating peptides are enriched by greater than 100% in one or more amino acids listed in FIG. 9B.

17. The method of claim 6, wherein said autoimmune disease is systemic lupus erythematosus, and wherein said different autoimmune disease is Sjogren's disease (SS).

18. The method of claim 17, wherein said discriminating peptides are enriched by greater than 100% in one or more sequence motifs listed in FIG. 10A.

19. The method of claim 17, wherein said discriminating peptides are enriched by greater than 100% in one or more amino acids listed in FIG. 10B.

20. The method of claim 6, wherein said autoimmune disease is systemic lupus erythematosus, and wherein said different disease is a group of autoimmune diseases and non-autoimmune mimic diseases.

21. The method of claim 20, wherein said discriminating peptides are enriched by greater than 100% in one or more sequence motifs listed in FIG. 5A.

22. The method of claim 20, wherein said discriminating peptides are enriched by greater than 100% in one or more amino acids listed in FIG. 5B.

23. The method of claim 6, wherein said autoimmune disease is systemic lupus erythematosus, and wherein said different disease is a group of autoimmune diseases, non-autoimmune mimic diseases, and healthy controls.

24. The method of claim 20, wherein said discriminating peptides are enriched by greater than 100% in one or more sequence motifs listed in FIG. 6A.

25. The method of claim 20, wherein said discriminating peptides are enriched by greater than 100% in one or more amino acids listed in FIG. 6B.

26. The method of claim 1, further comprising comparing the binding signal from subjects with systemic lupus erythematosus to a combination of reference binding signals obtained from healthy subjects.

27. The method of claim 26, wherein said discriminating peptides are enriched by greater than 100% in one or more sequence motifs listed in FIG. 4A.

28. The method of claim 26, wherein said discriminating peptides are enriched by greater than 100% in one or more amino acids listed in FIG. 4B.

29. The method of claim 6, wherein said autoimmune disease is rheumatoid arthritis (RA), and said different disease is OA.

30. The method of claim 29, wherein said discriminating peptides are enriched by greater than 100% in one or more sequence motifs listed in FIG. 22A.

31. The method of claim 29, wherein said discriminating peptides are enriched by greater than 100% in one or more amino acids listed in FIG. 22B.

32. The method of claim 6, wherein said autoimmune disease is rheumatoid arthritis (RA), and said different disease is FM.

33. The method of claim 32, wherein said discriminating peptides are enriched by greater than 100% in one or more sequence motifs listed in FIG. 23A.

34. The method of claim 32, wherein said discriminating peptides are enriched by greater than 100% in one or more amino acids listed in FIG. 23B.

35. The method of claim 6, wherein said autoimmune disease is rheumatoid arthritis (RA), and said different disease is SS.

36. The method of claim 35, wherein said discriminating peptides are enriched by greater than 100% in one or more sequence motifs listed in FIG. 24A.

37. The method of claim 35, wherein said discriminating peptides are enriched by greater than 100% in one or more amino acids listed in FIG. 24B.

38. The method of claim 6, wherein said autoimmune disease is RA, and wherein said different disease is a group of autoimmune diseases and non-autoimmune mimic diseases.

39. The method of claim 38, wherein said discriminating peptides are enriched by greater than 100% in one or more sequence motifs listed in FIG. 21A.

40. The method of claim 38, wherein said discriminating peptides are enriched by greater than 100% in one or more amino acids listed in FIG. 21B.

41. The method of claim 6, wherein said autoimmune disease is RA, and wherein said different disease is a group of autoimmune diseases, non-autoimmune mimic diseases, and healthy controls.

42. The method of claim 41, wherein said discriminating peptides are enriched by greater than 100% in one or more sequence motifs listed in FIG. 20A.

43. The method of claim 42, wherein said discriminating peptides are enriched by greater than 100% in one or more amino acids listed in FIG. 20B.

44. The method of claim 1, further comprising comparing the binding signal from subjects with RA to a combination of reference binding signals obtained from healthy subjects (HC).

45. The method of claim 44, wherein said discriminating peptides are enriched by greater than 100% in one or more sequence motifs listed in FIG. 18A.

46. The method of claim 44, wherein said discriminating peptides are enriched by greater than 100% in one or more amino acids listed in FIG. 18B.

47. The method of claim 1, further combining differentiating binding signals that distinguish samples from each of SLE, RA, FM, OA, SS and HC from each other to obtain a multiclass set of discriminating peptides that simultaneously distinguish each condition from each other.

48. The method of claim 47, wherein said discriminating peptides are enriched by greater than 100% in one or more sequence motifs listed in FIG. 30A.

49. The method of claim 44, wherein said discriminating peptides are enriched by greater than 100% in one or more amino acids listed in FIG. 30B.

50. The method of claim 1, wherein the method performance is characterized by an area under the receiver operator characteristic (ROC) curve (AUC) ranging from 0.60 to 0.70, 0.70 to 0.79, 0.80 to 0.89, or 0.90 to 1.00.

51. A method for identifying at least one candidate biomarker for an autoimmune disease, the method comprising:
(a) providing a peptide array and incubating a biological sample from a plurality of reference subjects known to have the autoimmune disease to the peptide array;
(b) identifying a set of discriminating peptides bound to antibodies in the biological sample from said subject, the set of discriminating peptides displaying binding signals capable of differentiating the autoimmune disease from samples from healthy subjects;
(c) querying a proteome database with each of the peptides in the set of discriminating peptides;
(d) aligning each of the peptides in the set of discriminating peptides to one or more proteins in the human proteome database; and
(e) obtaining a relevance score and ranking for each of the identified proteins from the proteome database;
wherein each of the identified proteins is a candidate biomarker for the autoimmune disease.

52. The method of claim 51, further comprising obtaining an overlap score, wherein said score corrects for the peptide composition of the peptide library.

53. The method of claim 51, wherein the step of identifying said set of discriminating peptides comprises:
(i) detecting the binding of antibodies present in samples form a plurality of subjects with the autoimmune disease to an array of different peptides to obtain a first combination of binding signals;
(ii) detecting the binding of antibodies to a same array of peptides, said antibodies being present in samples from two or more reference groups of subjects, each group being seronegative for said disease, to obtain a second combination of binding signals;
(iii) comparing said first to said second combination of binding signals; and
(iv) identifying said peptides on said array that are differentially bound by antibodies in samples from the subjects with the autoimmune disease from a reference groups of healthy subjects, thereby identifying said discriminating peptides.

54. The method of claim 51, wherein the number of discriminating peptides corresponds to at least a portion of the total number of peptides on said array.

55. The method of claim 51, wherein the autoimmune disease is systemic lupus erythematosus.

56. The method of claim 51, wherein said at least one candidate protein biomarker is selected from the list provided in FIG. 17A.

57. The method of claim 51, wherein the autoimmune disease is rheumatoid arthritis.

58. The method of claim 51, wherein said at least one candidate protein biomarker is selected from the list provided in FIG. 29A.

59. The method of any one of the claims above, wherein the sample is a blood sample.

60. The method of claim 56, wherein the blood sample is selected from whole blood, plasma, or serum

61. The method of any one of the claims above, wherein the sample is a serum sample.

62. The method of any one of the claims above, wherein the sample is a plasma sample.

63. The method of any one of the claims above, wherein the sample is a dried blood sample.

64. The method of any one of the claims above, wherein the array of peptides comprises at least 50,000 different peptides.

65. The method of any one of the claims above, wherein the peptide array comprises at least 300,000 different peptides.

66. The method of any one of the claims above, wherein the peptide array comprises at least 500,000 different peptides.

67. The method of any one of the claims above, wherein the peptide array comprises at least 2,000,000 different peptides.

68. The method of any one of the claims above, wherein the peptide array comprises at least 3,000,000 different peptides.

69. The method of any one of the claims above, wherein the different peptides on the peptide array is at least 5 amino acids in length.

70. The method of any one of the claims above, wherein the different peptides on the peptide array are between 5 and 13 amino acids in length.

71. The method of any one of the claims above, wherein the different peptides are synthesized from less than 20 amino acids.

72. The method of any one of the claims above, wherein the different peptides on the array are deposited.

73. The method of any one of the claims above, wherein the different peptides on the array are synthesized in situ.

74. A kit for making a differential diagnosis of an autoimmune disease, said kit comprising:
(a) an array of peptides comprising at least 10,000 different peptides for contacting a sample from a subject;
(b) a detection agent for detecting the binding of antibodies present in said sample to at least 25 peptides on said array to obtain a combination of binding signals; and
(c) a means for comparing said combination of binding signals to one or more groups of combination of reference binding signals, wherein at least one of said group of combination of reference binding signals are obtained from a plurality of reference subjects known to have a disease different from the autoimmune disease of the subject to enable the differential diagnosis of said subject for the autoimmune disease, wherein the method performance is characterized by an area under the receiver operator characteristic (ROC) curve (AUC) between the autoimmune disease and each of the group of combinations of reference binding signals being greater than 0.6.

75. The kit of claim 74, further comprising:
(i) means for identifying a combination of differentiating reference binding signals, wherein said differentiating reference binding signals distinguish samples from reference subjects known to have said autoimmune disease from samples from reference subjects known to have said disease different from the autoimmune disease; and
(ii) means for applying the combination of differentiating reference binding signals to step 1(c) to enable differential diagnosis of the autoimmune disease.

76. The kit of claim 75, wherein each of said combination of differentiating reference binding signals is obtained by detecting the binding of antibodies present in a sample from each of said plurality of reference subjects to said at least 25 peptides on an array of peptides comprising at least 10,000 different peptides in step (a) of claim 1.

77. The kit of claim 75, wherein the difference between said combination of binding signals and said combination of said reference binding signals to said at least 25 peptides determines said differential diagnosis.

78. The kit of claim 74, further comprising:
(d) means for comparing said combination of binding signals to a reference binding signal obtained from a plurality of reference subjects known to have the autoimmune disease.

79. The kit of claim 74, wherein said different disease is an autoimmune disease.

80. The kit of claim 74, wherein said different disease is a non-autoimmune mimic disease.

81. The kit of claim 79, wherein said autoimmune disease is systemic lupus erythematosus, and wherein said different autoimmune disease is rheumatoid arthritis.

82. The kit of claim 81, wherein said discriminating peptides are enriched by greater than 100% in one or more sequence motifs listed in FIG. 7A.

83. The kit of claim 81, wherein said discriminating peptides are enriched by greater than 100% in one or more amino acids listed in FIG. 7B.

84. The kit of claim 79, wherein said autoimmune disease is systemic lupus erythematosus, and wherein said different non-autoimmune-disease is osteoarthritis.

85. The kit of claim 84, wherein said discriminating peptides are enriched by greater than 100% in one or more sequence motifs listed in FIG. 8A.

86. The kit of claim 84, wherein said discriminating peptides are enriched by greater than 100% in one or more amino acids listed in FIG. 8B.

87. The kit of claim 79, wherein said autoimmune disease is systemic lupus erythematosus, and wherein said different non-autoimmune disease is fibromyalgia.

88. The kit of claim 87, wherein said discriminating peptides are enriched by greater than 100% in one or more sequence motifs listed in FIG. 9A.

89. The kit of claim 87, wherein said discriminating peptides are enriched by greater than 100% in one or more amino acids listed in FIG. 9B.

90. The kit of claim 79, wherein said autoimmune disease is systemic lupus erythematosus, and wherein said different autoimmune disease is Sjogren's disease.

91. The kit of claim 90, wherein said discriminating peptides are enriched by greater than 100% in one or more sequence motifs listed in FIG. 10A.

92. The kit of claim 90, wherein said discriminating peptides are enriched by greater than 100% in one or more amino acids listed in FIG. 10B.

93. The kit of claim 79, wherein said autoimmune disease is systemic lupus erythematosus, and wherein said different disease is a group of autoimmune diseases and non-autoimmune mimic diseases.

94. The kit of claim 93, wherein said discriminating peptides are enriched by greater than 100% in one or more sequence motifs listed in FIG. 5A.

95. The kit of claim 93, wherein said discriminating peptides are enriched by greater than 100% in one or more amino acids listed in FIG. 5B.

96. The kit of claim 79, wherein said autoimmune disease is systemic lupus erythematosus, and wherein said different disease is a group of autoimmune diseases, non-autoimmune mimic diseases, and healthy controls.

97. The kit of claim 96, wherein said discriminating peptides are enriched by greater than 100% in one or more sequence motifs listed in FIG. 6A.

98. The kit of claim 96, wherein said discriminating peptides are enriched by greater than 100% in one or more amino acids listed in FIG. 6B.

99. The kit of claim 74, further comprising means for comparing the binding signal from subjects with systemic lupus erythematosus to a combination of reference binding signals obtained from healthy subjects.

100. The kit of claim 99, wherein said discriminating peptides are enriched by greater than 100% in one or more sequence motifs listed in FIG. 4A.

101. The kit of claim 99, wherein said discriminating peptides are enriched by greater than 100% in one or more amino acids listed in FIG. 4B.

102. The kit of claim 79, wherein said autoimmune disease is rheumatoid arthritis (RA), and said different disease is OA.

103. The kit of claim 102, wherein said discriminating peptides are enriched by greater than 100% in one or more sequence motifs listed in FIG. 22A.

104. The kit of claim 102, wherein said discriminating peptides are enriched by greater than 100% in one or more amino acids listed in FIG. 22B.

105. The kit of claim 79, wherein said autoimmune disease is rheumatoid arthritis (RA), and said different disease is FM.

106. The kit of claim 105, wherein said discriminating peptides are enriched by greater than 100% in one or more sequence motifs listed in FIG. 23A.

107. The kit of claim 105, wherein said discriminating peptides are enriched by greater than 100% in one or more amino acids listed in FIG. 23B.

108. The kit of claim 79, wherein said autoimmune disease is rheumatoid arthritis (RA), and said different disease is SS.

109. The kit of claim 108, wherein said discriminating peptides are enriched by greater than 100% in one or more sequence motifs listed in FIG. 24A.

110. The kit of claim 108, wherein said discriminating peptides are enriched by greater than 100% in one or more amino acids listed in FIG. 24B.

111. The kit of claim 79, wherein said autoimmune disease is RA, and wherein said different disease is a group of autoimmune diseases and non-autoimmune mimic diseases.

112. The kit of claim 111, wherein said discriminating peptides are enriched by greater than 100% in one or more sequence motifs listed in FIG. 21A.

113. The kit of claim 111, wherein said discriminating peptides are enriched by greater than 100% in one or more amino acids listed in FIG. 21B.

114. The kit of claim 79, wherein said autoimmune disease is RA, and wherein said different disease is a group of autoimmune diseases, non-autoimmune mimic diseases, and healthy controls.

115. The kit of claim 114, wherein said discriminating peptides are enriched by greater than 100% in one or more sequence motifs listed in FIG. 20A.

116. The kit of claim 114, wherein said discriminating peptides are enriched by greater than 100% in one or more amino acids listed in FIG. 20B.

117. The kit of claim 74, further comprising means for comparing the binding signal from subjects with RA to a combination of reference binding signals obtained from healthy subjects (HC).

118. The kit of claim 117, wherein said discriminating peptides are enriched by greater than 100% in one or more sequence motifs listed in FIG. 18A.

119. The kit of claim 117, wherein said discriminating peptides are enriched by greater than 100% in one or more amino acids listed in FIG. 18B.

120. The kit of claim 74, further means for combining differentiating binding signals that distinguish samples from each of SLE, RA, FM, OA, SS and HC from each other to obtain a multiclass set of discriminating peptides that simultaneously distinguish each condition from each other.

121. The kit of claim 120, wherein said discriminating peptides are enriched by greater than 100% in one or more sequence motifs listed in FIG. 30A.

122. The kit of claim 120, wherein said discriminating peptides are enriched by greater than 100% in one or more amino acids listed in FIG. 30B.

123. The kit of claim 74, wherein the method performance is characterized by an area under the receiver operator characteristic (ROC) curve (AUC) ranging from 0.60 to 0.70, 0.70 to 0.79, 0.80 to 0.89, or 0.90 to 1.00.

124. A kit for identifying at least one candidate biomarker for an autoimmune disease, the kit comprising:
(a) a peptide array for incubating a biological sample from a plurality of reference subjects known to have the autoimmune disease;
(b) means for identifying a set of discriminating peptides bound to antibodies in the biological sample from said subject, the set of discriminating peptides displaying binding signals capable of differentiating the autoimmune disease from samples from healthy subjects;
(c) means for querying a proteome database with each of the peptides in the set of discriminating peptides;
(d) means for aligning each of the peptides in the set of discriminating peptides to one or more proteins in the human proteome database; and
(e) means for obtaining a relevance score and ranking for each of the identified proteins from the proteome database;
wherein each of the identified proteins is a candidate biomarker for the autoimmune disease.

125. The kit of claim 124, further comprising means for obtaining an overlap score, wherein said score corrects for the peptide composition of the peptide library.

126. The kit of claim 124, wherein the means for identifying said set of discriminating peptides comprises:
(i) means for detecting the binding of antibodies present in samples form a plurality of subjects with the autoimmune disease to an array of different peptides to obtain a first combination of binding signals;
(ii) means for detecting the binding of antibodies to a same array of peptides, said antibodies being present in samples from two or more reference groups of subjects, each group being seronegative for said disease, to obtain a second combination of binding signals;
(iii) means for comparing said first to said second combination of binding signals; and
(iv) means for identifying said peptides on said array that are differentially bound by antibodies in samples from the subjects with the autoimmune disease from a reference groups of healthy subjects, thereby identifying said discriminating peptides.

127. The kit of claim 124, wherein the number of discriminating peptides corresponds to at least a portion of the total number of peptides on said array.

128. The kit of claim 124, wherein the autoimmune disease is systemic lupus erythematosus.

129. The kit of claim 124, wherein said at least one candidate protein biomarker is selected from the list provided in FIG. 17A.

130. The kit of claim 124, wherein the autoimmune disease is rheumatoid arthritis.

131. The kit of claim 51, wherein said at least one candidate protein biomarker is selected from the list provided in FIG. 29A.

132. The kit of any one of the claims above, wherein the sample is a blood sample.

133. The kit of claim 132, wherein the blood sample is selected from whole blood, plasma, or serum

134. The kit of any one of the claims above, wherein the sample is a serum sample.

135. The kit of any one of the claims above, wherein the sample is a plasma sample.

136. The kit of any one of the claims above, wherein the sample is a dried blood sample.

137. The kit of any one of the claims above, wherein the array of peptides comprises at least 50,000 different peptides.

138. The kit of any one of the claims above, wherein the peptide array comprises at least 300,000 different peptides.

139. The kit of any one of the claims above, wherein the peptide array comprises at least 500,000 different peptides.

140. The kit of any one of the claims above, wherein the peptide array comprises at least 2,000,000 different peptides.

141. The kit of any one of the claims above, wherein the peptide array comprises at least 3,000,000 different peptides.

142. The kit of any one of the claims above, wherein the different peptides on the peptide array is at least 5 amino acids in length.

143. The kit of any one of the claims above, wherein the different peptides on the peptide array are between 5 and 13 amino acids in length.

144. The kit of any one of the claims above, wherein the different peptides are synthesized from less than 20 amino acids.

145. The kit of any one of the claims above, wherein the different peptides on the array are deposited.

146. The kit of any one of the claims above, wherein the different peptides on the array are synthesized in situ.
